(19) United States
(12) Patent Application Publication
(10) Pub. No.: US 2026/0337262 A1
(43) Pub. Date: Oct. 29, 2026

(54) Methods of Treating Idiopathic Inflammatory Myopathies with IFNß Antibodies

(71) Applicant: PFIZER INC., New York, NY (US)

(72) Inventors: Shilpa Sanjay ALEKAR, San Diego, CA (US); Anindita BANERJEE, Cambridge, MA (US); Christopher Ricardo BANFIELD, Woburn, MA (US); Jean Saccuzzo BEEBE; Myron Edward CHU, Downingtown, PA (US); Craig Lee HYDE, Old Lyme, CT (US); Kristen Leigh JOHNSON, Cambridge, MA (US); Xingpeng LI, Acton, MA (US); Elena PEEVA, Tarrytown, NY (US); Barry Setiawan OEMAR, Cambridge, MA (US); Jean Natalie RATH, Conshohocken, PA (US); Mikhail Peter SALGANIK, Boston, MA (US); Abigail Grace SLOAN, Brookline, MA (US); Daniel ZIEMEK, Berlin (DE); Abhinav TIWARI, Boston, MA (US); Liji JOSEPH, Chesterfield, MO (US); Chandra Anne WEBB, Windham, NH (US); Srividya NEELAKANTAN, East Walpole, MA (US)

(21) Appl. No.: 19/138,219

(22) PCT Filed: Sep. 21, 2023
(86) PCT No.: PCT/IB2023/059364

§ 371 Date: Jun. 12, 2025

Related U.S. Application Data
(60) Provisional application No. 63/376,722, filed on Sep. 22, 2022.
(60) Provisional application No. 63/490,120, filed on Mar. 14, 2023.
(60) Provisional application No. 63/583,336, filed on Sep. 18, 2023.

(51) Int. Cl. C07K 16/24; A61K 9/08; A61K 39/00; A61K 47/18; A61K 47/22; A61K 47/26; A61P 17/00; A61P 21/00; A61P 37/02

(52) U.S. Cl. CPC C07K 16/249; A61K 9/08; A61K 47/183; A61K 47/22; A61K 47/26; A61P 17/00; A61P 21/00; A61P 37/02; A61K 2039/505; A61K 2039/545; C07K 2317/24; C07K 2317/76

(57) ABSTRACT

The invention provides methods for treating a patient with one or more conditions associated with IFNß expression in a patient, the method comprising administering to the patient an anti-IFNß antibody in a dosing regimen sufficient to improve signs and symptoms of the one or more conditions by at least 4 weeks after the start of treatment with the anti-IFNß antibody, said dosing regimen comprising a plurality of individual doses separated from each other by at least 1 weeks, as well as formulations suitable for such methods.

### FIELD

The present invention relates to the treatment of signs and symptoms of idiopathic inflammatory myopathies with an anti-interferon beta (IFNß) antibody.

### BACKGROUND

Idiopathic inflammatory myopathies (IIM) are an heterogenous group of rare and chronic, autoimmune diseases characterized by muscle inflammation, muscle weakness and extra-muscular manifestations including involvement of the skin, lung, heart, GI tract and joints. The most common types of IIM in adults are Dermatomyositis (DM), Polymyositis (PM), inclusion body myositis (IBM), and Juvenile forms of DM (JDM) in children. (Malik et al, 2016). Patients diagnosed with IIM are often classified into subtypes that allow clinicians to predict prognosis, individualize treatment plans and other therapeutic approaches (Loarce-Martos et al, 2021). The Bohan and Peter criteria, published in 1975, has been commonly used to classify DM and PM patients. Since then and given the discovery and use of autoantibody testing and identification of distinct subgroups; newer classification criteria have been developed (Baig & Paik, 2020). A validated classification criteria was approved by ACR and EULAR in 2017 and is currently widely used and accepted to classify IIM patients (Lundberg et al, 2017). These criteria classify patients as having “definite”, “probable”, “possible” and “non-IIM” based on a score and corresponding probability of disease. Subclassification of IIM distinguished between adult and juvenile myositis, polymyositis, dermatomyositis, amyopathic DM or Inclusion Body Myositis

DM is a rare, serious, severely debilitating autoimmune disease involving both neuromuscular and cutaneous manifestations; the disease is characterized by inflammation of skeletal muscle and skin, with concomitant skeletal muscle weakness and a distinctive severe skin rash (Dalakas, 1991). Skin manifestations may occur in up to 94% of patients with DM and often precede or accompany muscle weakness (Ahmed et al, 2020a). Muscle weakness is also a common feature in >80% of DM, with patients exhibiting proximal skeletal muscle weakness and elevated serum muscle enzymes, eg, creatinine kinase and aldolase (Bohan & Peter, 1975a; Bohan & Peter, 1975b; Dalakas & Hohlfeld, 2003; Findlay et al, 2015). Symptoms can come on suddenly or gradually over time and often wax and wane for no apparent reason. DM is also associated with an overall increased risk of malignancy, in particular ovarian, lung, pancreatic, stomach and colorectal cancers, and reduced life expectancy (Oldroyd et al, 2021; Vaughan et al, 2022). Other clinical manifestations associated with DM include calcinosis, cardiac abnormalities (including arrhythmia, congestive heart failure, myocarditis, pericarditis, angina, and fibrosis), dysphagia (difficulty in swallowing), polyarthritis, and interstitial lung disease (Na et al, 2009; Khan & Christopher-Stine, 2011).

Juvenile DM (JDM) is a rare, often chronic autoimmune disease with onset during childhood. It is characterized by weakness in proximal muscles and pathognomonic skin rashes. Although the etiology remains unclear, it is proposed that JDM is caused by a vasculopathy within the muscle tissue and multiple other organ systems of genetically susceptible individuals in response to environmental triggers. Delayed treatment of JDM can lead to poorer outcomes in terms of disease course and calcinosis (Batthish & Feldman, 2011). There are some differences between juvenile and adult DM with respect to prevalence of features, outcomes and comorbidities. Children with DM have more vasculopathy, calcinosis, periungual and gingival telangiectasias, and ulceration, but have a better long-term prognosis with improved survival compared to adult DM. Adults with DM are more likely to have myositis-specific antibodies, develop ILD, have amyopathic disease, and marked association with malignancy (Oldroyd et al, 2021; Vaughan et al, 2022) and other comorbidities (Robinson & Reed, 2011).

Polymyositis (PM) is a rare disease affecting mainly adults. The multiple classification criteria of IIM that have been used since Bohan and Peter classification criteria was published have a common limitation which is that they do not always exclude other types of myopathy and misclassify IBM patients as PM. The subgrouping of patients based on clinical manifestations, histopathological findings and the presence of MSA may improve precision of classification. Although the use of MSAs has increased, a classification criterion based on these has not been validated. Per the ACR/EULAR criteria, patients classified as PM may have autoantibodies associated with Antisynthetase syndrome and immune-mediated necrotizing myopathy (Lundberg et al, 2017). This group may also include patients with no specific autoantibodies but with clinical manifestations corresponding to PM. PM manifests primarily in the muscle, with characteristic proximal muscle weakness, elevated muscle enzymes and myopathy features on electromyography. Dysphagia may occur in a subset of patients. The most commonly associated extra-muscular manifestations are interstitial lung disease and cardiac involvement. The skin manifestations of DM are absent in PM patients (Malik et al, 2016; Baig & Paik, 2020).

In terms of epidemiology, DM affects both adults and children (Findlay et al, 2015). DM occurs most often after the age of 40 years (Bogdanov et al, 2018) and females are affected twice as often as males (NORD, 2015). Juvenile DM is the most common inflammatory myositis in children and is observed most often between the ages of 5 to 12 years (Dourmishev A L, 2009). PM primarily affects adults, with a ratio of adult-to-child cases greater than that observed in DM (Shah et al, 2013). PM is also more prevalent in females than males, with estimates ranging from 50% more likely in females (Svensson et al, 2017) to twice as likely (See et al, 2013). Juvenile PM is much less prevalent (greater than 10-fold) than JDM. The average age at JPM diagnosis is 12.1 years (Shah et al, 2013) compared with the median age of JDM diagnosis of 7.4 years.

From the patient perspective, the common clinical manifestation across all types of IIM is symmetric proximal muscle weakness. Many patients with IIM have extra-muscular symptoms as part of their initial presenting clinical manifestations. These include skin rash, particularly in DM patients, arthritis and respiratory involvement in the form of ILD. For patients with DM and JDM, the most problematic signs and symptoms include 2 general categorizations—skin symptoms and muscle symptoms. Physical symptoms for skin include: rash, itch, pain, soreness; skin rash (itchy and/or painful) is often the first sign of DM (Cleland & Venzke, 2003). Common muscle-involved complaints of patients with DM include fatigue, tiredness, muscle weakness, and reduced endurance (Okogbaa & Batiste, 2019). For patients with PM, proximal muscle weakness, sometimes accompanied with pain is the main clinical manifestations. Patients have difficulty climbing steps, getting up from a seated position or raising their arms above their head (Lundberg et al, 2021).

Like other autoimmune disorders, it is thought that the interaction between genetic and environmental factors results in clinical phenotypes of IIM. The exact process and triggers for the immune system are not well understood, although there is evidence that both the adaptive and innate immune mechanisms and non-immune mechanisms are involved in the different types of IIM (Lundberg et al, 2021). There is literature data that suggests various risk factors, for example the presence of certain autoantibodies, resulting in different clinical features, response to treatment and outcomes. The pathogenesis in DM and JDM is multifactorial and complex. A strong genetic component has been described in DM and JDM. Data from genotyping studies has suggested association of major histocompatibility complex polymorphism and the development of DM in adults and children. There is also evidence that show particular HLA alleles associated with autoantibody production which correlates with clinical phenotypes (DeWane et al, 2020). There are multiple environmental factors that may trigger immune activation. Some of the triggers described in patients with DM and JDM include UV radiation, viral infections, medications and smoking. Similar to DM and JDM, there are genetic and environmental factors that contribute to the development of clinical PM.

Treatment of PM or DM is a difficult task in large part due to its rarity, its multiple clinical phenotypes, and the fact that the disease affects multiple organ systems and is commonly treatment-refractory (Bogdanov et al, 2018). Neither PM nor DM have a known cure and despite the significant morbidity and mortality associated with the condition, there is currently only 1 pharmacologic therapy (Octagam 10% [IVIG]) that was recently approved to treat DM in the US and EU on the basis of randomized controlled trials. Treatment of PM or DM is a difficult task in large part due to its rarity, its multiple clinical phenotypes, and the fact that the disease affects multiple organ systems and is commonly treatment-refractory (Bogdanov et al, 2018). The choice of treatment or the sequence in which various immunotherapeutic drugs are used is not evidence-based but rather is empirical and often influenced by physician experience, prejudice and the treating physician's personal perception of the efficacy/safety ratio of a given therapy (Dalakas, 2010).

For either PM or DM, the mainstay of therapy is a combination of immunosuppressive drugs to treat the inflammatory symptoms. The first line drug treatment for the muscle disease is typically systemic corticosteroids (eg, high dose systemic prednisolone) to address the inflammation as well as suppress the immune system (Dalakas, 2010; Dalakas, 2011).

Other immunosuppressive drugs, notably azathioprine, methotrexate, MMF, hydroxychloroquine, cyclosporine and cyclophosphamide are used as subsequent lines of therapies in refractory cases or as steroid-sparing agents (Findlay et al, 2015). The preference for these drugs is empirical and not evidence-based and their use as a sole treatment seems to provide little benefit (Findlay et al, 2015). Furthermore, these agents are also associated with common and significant toxicities, including thrombocytopenia, anemia, leukopenia and pancytopenia (azathioprine), liver and bone marrow toxicities (methotrexate, cyclosporine, cyclophosphamide), kidney (cyclosporine), and gastro-intestinal symptoms and leukopenia (MMF) (Dalakas, 2010). Despite their empiric use, evidence from randomized clinical trials for these therapies remain limited (Fasano et al, 2016). Complications of long-term corticosteroid and other immunosuppressive therapies are well documented (Dalakas, 2010; Bradford Rice et al, 2016; Oray et al, 2016; Rice et al, 2017). Azathioprine and some corticosteroid products (eg, prednisolone, betamethasone, dexamethasone) have been nationally authorized in some EU countries for the treatment of IIMs and a range of related conditions. A branded generic of azathioprine (Jayempi) was recently approved in the EU via the centralized procedure for the treatment of a number of inflammatory conditions, including DM (but not PM) in patients who are intolerant to glucocorticosteroids or if the therapeutic response is inadequate despite treatment with high doses of glucocorticosteroids (EMA, 2021). However, it would appear that these approvals were based primarily on empiric data and literature reports rather than data from controlled clinical trials (as noted above). These treatments are typically used in combination with other drugs and procedures.

Other approaches have emerged as potential treatments, including tacrolimus (a broadly immunosuppressive drug developed for transplant use), IVIg, and rituximab (anti-CD20 mAb), following positive outcomes in some small case studies (Fasano et al, 2016); however, the safety profile of these agents is also challenging to manage in the clinic (Dalakas, 2010; Fasano et al, 2016). Octagam 10% (IVIg) was recently approved in the EU (first approval in Germany in May 2021) via the decentralised procedure for the treatment of adults with active DM treated with immunosuppressive drugs, including corticosteroids, or with intolerance or contraindications to those drugs (Octapharma press release, June 2021), and in the US (in July 2021) for the treatment of DM in adults. These approvals were based on the results from a single Phase 3 randomised controlled trial of IVIg for dermatomyositis (ProDERM study; NCT02728752). It should be noted that Octagam is associated with a number of safety risks including thrombosis, renal dysfunction and acute renal failure, and the posology which requires administration via 2-5 IV infusions with high doses over consecutive days each month represents an additional burden for patients. Octagam is not currently approved for PM.

The first-line therapies for JDM patients are typically corticosteroids and/or methotrexate. Second line therapies are IVIg, rituximab, cyclosporin, azathioprine, tacrolimus and mycophenolate mofetil. Third-line therapy is stem cell transplant or cyclophosphamide (Robinson & Reed, 2011). Octagam is not currently approved for treatment of JDM.

Moreover, even with current treatments, substantial numbers of patients remain treatment-refractory. The currently used treatments are also associated with significant safety concerns. Recently, Octagam (10% IVIg) was approved in the US and EU for the treatment of adult DM; it is not approved for JDM or PM. Octagam is associated with several risks (eg, thrombosis, renal dysfunction, acute renal failure) and has a posology that requires infusions over several consecutive days, each month. As such, IlM (DM, PM and JDM) represent a major unmet need for effective and safe therapies and there therefore exists a need for additional treatments for Idiopathic inflammatory myopathies.

### SUMMARY OF THE INVENTION

The invention provides methods of treating a patient with a condition associated with aberrant levels of IGFß expression comprising administering an anti-IFNß antibody. The invention further provides methods of treating a patient with idiopathic inflammatory myopathy (IIM) comprising administering an anti-IFNß antibody. Those skilled in the art will recognize or be able to ascertain using no more than routine experimentation, many equivalents to the specific embodiments of the invention described herein. Such equivalents are intended to be encompassed by the following embodiments (E).
E1. In a first embodiment, the invention relates to a method for treating a patient with one or more conditions associated with IFNß expression in a patient, the method comprising administering to the patient an anti-IFNß antibody in a dosing regimen sufficient to improve signs and symptoms of the one or more conditions by at least 4 weeks after the start of treatment with the anti-IFNß antibody, said dosing regimen comprising a plurality of individual doses separated from each other by at least 4 weeks.
E2. In another embodiment, the invention relates to a method for treating a patient with one or more conditions selected from the group consisting of IIM, dermatomyositis, polymyositis, inclusion body myositis, SLE, Cutaneous Lupus, Psoriasis, Ulcerative Colitis, Crohn's Disease, Rheumatoid Arthritis, Atopic Dermatitis and Scleroderma in a patient, the method comprising administering to the patient an anti-IFNß antibody in a dosing regimen sufficient to improve signs and symptoms of the one or more condition by at least 4 weeks after the start of treatment with the anti-IFNß antibody, said dosing regimen comprising a plurality of individual doses separated from each other by at least 4 weeks.
E3. In another embodiment, the invention relates to a method for treating one or more conditions selected from the group consisting of IIM, dermatomyositis, polymyositis, inclusion body myositis, SLE, and Cutaneous Lupus in a patient, the method comprising administering to the patient an anti-IFNß antibody in a dosing regimen sufficient to improve signs and symptoms of IIM by at least 4 weeks after the start of treatment with the anti-IFNß antibody, said dosing regimen comprising a plurality of individual doses separated from each other by at least 4 weeks.
E4. In a first embodiment, the invention relates to a method for treating IIM in a patient, the method comprising administering to the patient an anti-IFNß antibody in a dosing regimen sufficient to improve signs and symptoms of IIM by at least 4 weeks after the start of treatment with the anti-IFNß antibody, said dosing regimen comprising a plurality of individual doses separated from each other by at least 4 weeks.
E5. In another embodiment, the invention relates to a method for treating dermatomyositis in a patient, the method comprising administering to the patient an anti-IFNß antibody in a dosing regimen sufficient to improve signs and symptoms of dermatomyositis by at least 4 weeks after the start of treatment with the anti-IFNß antibody, said dosing regimen comprising a plurality of individual doses separated from each other by at least 4 weeks.
E6. In another embodiment, the invention relates to a method for treating polymyositis in a patient, the method comprising administering to the patient an anti-IFNß antibody in a dosing regimen sufficient to improve signs and symptoms of polymyositis by at least 4 weeks after the start of treatment with the anti-IFNß antibody, said dosing regimen comprising a plurality of individual doses separated from each other by at least 4 weeks.
E7. In another embodiment, the invention relates to a method for treating inclusion body myositis in a patient, the method comprising administering to the patient an anti-IFNß antibody in a dosing regimen sufficient to improve signs and symptoms of inclusion body myositis by at least 4 weeks after the start of treatment with the anti-IFNß antibody, said dosing regimen comprising a plurality of individual doses separated from each other by at least 4 weeks.
E8. In another embodiment, the invention relates to a method for treating a patient with one or more conditions selected from the group consisting of SLE and Cutaneous Lupus, the method comprising administering to the patient an anti-IFNß antibody in a dosing regimen sufficient to improve signs and symptoms of the one or more condition by at least 4 weeks after the start of treatment with the anti-IFNß antibody, said dosing regimen comprising a plurality of individual doses separated from each other by at least 4 weeks.
E9. In another embodiment, the invention relates to a method for treating a patient with one or more conditions associated with IFNß expression in a patient, the method comprising administering to the patient an anti-IFNß antibody in a dosing regimen sufficient to improve signs and symptoms of the one or more conditions by at least 4 weeks after the start of treatment with the anti-IFNß antibody, said dosing regimen comprising a plurality of individual doses separated from each other by at least 1 week.
E10. In another embodiment, the invention relates to a method for treating a patient with one or more conditions selected from the group consisting of IIM, dermatomyositis, polymyositis, inclusion body myositis, SLE, Cutaneous Lupus, Psoriasis, Ulcerative Colitis, Crohn's Disease, Rheumatoid Arthritis, Atopic Dermatitis and Scleroderma in a patient, the method comprising administering to the patient an anti-IFNß antibody in a dosing regimen sufficient to improve signs and symptoms of the one or more condition by at least 4 weeks after the start of treatment with the anti-IFNß antibody, said dosing regimen comprising a plurality of individual doses separated from each other by at least 1 week.
E11. In another embodiment, the invention relates to a method for treating one or more conditions selected from the group consisting of IIM, dermatomyositis, polymyositis, inclusion body myositis, SLE, and Cutaneous Lupus in a patient, the method comprising administering to the patient an anti-IFNß antibody in a dosing regimen sufficient to improve signs and symptoms of IIM by at least 4 weeks after the start of treatment with the anti-IFNß antibody, said dosing regimen comprising a plurality of individual doses separated from each other by at least 1 week.
E12. In a first embodiment, the invention relates to a method for treating IIM in a patient, the method comprising administering to the patient an anti-IFNß antibody in a dosing regimen sufficient to improve signs and symptoms of IIM by at least 4 weeks after the start of treatment with the anti-IFNß antibody, said dosing regimen comprising a plurality of individual doses separated from each other by at least 1 week.
E13. In another embodiment, the invention relates to a method for treating dermatomyositis in a patient, the method comprising administering to the patient an anti-IFN& antibody in a dosing regimen sufficient to improve signs and symptoms of dermatomyositis by at least 4 weeks after the start of treatment with the anti-IFNß antibody, said dosing regimen comprising a plurality of individual doses separated from each other by at least 1 week.
E14. In another embodiment, the invention relates to a method for treating polymyositis in a patient, the method comprising administering to the patient an anti-IFN& antibody in a dosing regimen sufficient to improve signs and symptoms of polymyositis by at least 4 weeks after the start of treatment with the anti-IFNß antibody, said dosing regimen comprising a plurality of individual doses separated from each other by at least 1 week.
E15. In another embodiment, the invention relates to a method for treating inclusion body myositis in a patient, the method comprising administering to the patient an anti-IFNß antibody in a dosing regimen sufficient to improve signs and symptoms of inclusion body myositis by at least 4 weeks after the start of treatment with the anti-IFNß antibody, said dosing regimen comprising a plurality of individual doses separated from each other by at least 1 week.
E16. In another embodiment, the invention relates to a method for treating a patient with one or more conditions selected from the group consisting of SLE and Cutaneous Lupus, the method comprising administering to the patient an anti-IFNß antibody in a dosing regimen sufficient to improve signs and symptoms of the one or more condition by at least 4 weeks after the start of treatment with the anti-IFNß antibody, said dosing regimen comprising a plurality of individual doses separated from each other by at least 1 week.
E17. In another embodiment, the invention relates to a method for treating a patient with one or more conditions associated with IFNß expression in a patient, the method comprising administering to the patient an anti-IFNß antibody in a dosing regimen sufficient to improve signs and symptoms of the one or more conditions by at least 4 weeks after the start of treatment with the anti-IFNß antibody, said dosing regimen comprising a plurality of individual doses separated from each other by at least 2 weeks.
E18. In another embodiment, the invention relates to a method for treating a patient with one or more conditions selected from the group consisting of IIM, dermatomyositis, polymyositis, inclusion body myositis, SLE, Cutaneous Lupus, Psoriasis, Ulcerative Colitis, Crohn's Disease, Rheumatoid Arthritis, Atopic Dermatitis and Scleroderma in a patient, the method comprising administering to the patient an anti-IFNß antibody in a dosing regimen sufficient to improve signs and symptoms of the one or more condition by at least 4 weeks after the start of treatment with the anti-IFNß antibody, said dosing regimen comprising a plurality of individual doses separated from each other by at least 2 weeks.
E19. In another embodiment, the invention relates to a method for treating one or more conditions selected from the group consisting of IIM, dermatomyositis, polymyositis, inclusion body myositis, SLE, and Cutaneous Lupus in a patient, the method comprising administering to the patient an anti-IFNß antibody in a dosing regimen sufficient to improve signs and symptoms of IIM by at least 4 weeks after the start of treatment with the anti-IFNß antibody, said dosing regimen comprising a plurality of individual doses separated from each other by at least 2 weeks.
E20. In a first embodiment, the invention relates to a method for treating IIM in a patient, the method comprising administering to the patient an anti-IFNß antibody in a dosing regimen sufficient to improve signs and symptoms of IIM by at least 4 weeks after the start of treatment with the anti-IFNß antibody, said dosing regimen comprising a plurality of individual doses separated from each other by at least 2 weeks.
E21. In another embodiment, the invention relates to a method for treating dermatomyositis in a patient, the method comprising administering to the patient an anti-IFNß antibody in a dosing regimen sufficient to improve signs and symptoms of dermatomyositis by at least 4 weeks after the start of treatment with the anti-IFNß antibody, said dosing regimen comprising a plurality of individual doses separated from each other by at least 2 weeks.
E22. In another embodiment, the invention relates to a method for treating polymyositis in a patient, the method comprising administering to the patient an anti-IFNß antibody in a dosing regimen sufficient to improve signs and symptoms of polymyositis by at least 4 weeks after the start of treatment with the anti-IFNß antibody, said dosing regimen comprising a plurality of individual doses separated from each other by at least 2 weeks.
E23. In another embodiment, the invention relates to a method for treating inclusion body myositis in a patient, the method comprising administering to the patient an anti-IFNß antibody in a dosing regimen sufficient to improve signs and symptoms of inclusion body myositis by at least 4 weeks after the start of treatment with the anti-IFNß antibody, said dosing regimen comprising a plurality of individual doses separated from each other by at least 2 weeks.
E24. In another embodiment, the invention relates to a method for treating a patient with one or more conditions selected from the group consisting of SLE and Cutaneous Lupus, the method comprising administering to the patient an anti-IFNß antibody in a dosing regimen sufficient to improve signs and symptoms of the one or more condition by at least 4 weeks after the start of treatment with the anti-IFNß antibody, said dosing regimen comprising a plurality of individual doses separated from each other by at least 2 weeks.
E25. The method as set forth in E1-E24, wherein one or more of the individual doses are administered at least 1 month apart.
E26. The method as set forth in E1-E25, wherein one or more of the individual doses are administered at least 8 weeks apart.
E27. The method as set forth in E1-E26, wherein one or more of the individual doses are administered at least 2 months apart.
E28. The method as set forth in E1-E27, wherein one or more of the individual doses are at an amount of between 25 mg and 1000 mg.
E29. The method as set forth in E1-E28, wherein one or more of the individual doses are at an amount of between 150 mg and 600 mg.
E30. The method as set forth in E1-E29, wherein one or more of the individual doses are at an amount selected from the group consisting of 25 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, and 1000 mg.
E31. The method as set forth in E1-E30, wherein one or more of the individual doses are at an amount within a range whose lower limit is selected from the group consisting of 25 mg, 50 mg, 100 mg, 150 mg, 200 mg, 250 mg, 300 mg, 350 mg, 400 mg, 450 mg, 500 mg, 550 mg, and 600 mg.
E32. The method as set forth in E1-E31, wherein one or more of the individual doses are at an amount within a range whose upper limit is selected from the group consisting of 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, and 1000 mg.
E33. The method as set forth in E1-E32, wherein one or more of the individual doses are at an amount within a range whose lower limit is selected from the group consisting of 25 mg, 50 mg, 100 mg, 150 mg, 200 mg, 250 mg, 300 mg, 350 mg, 400 mg, 450 mg, 500 mg, 550 mg, and 600 mg, and whose upper limit is selected from the group consisting of 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, and 1000 mg.
E34. The method as set forth in E1-E33, wherein one or more of the individual doses are at an amount of at least 140 mg.
E35. The method as set forth in E1-E34, wherein one or more of the individual doses are at an amount of at least 150 mg.
E36. The method as set forth in E1-E35, wherein one or more of the individual doses are at an amount of at least 200 mg.
E37. The method as set forth in E1-E36, wherein one or more of the individual doses are at an amount of at least 250 mg.
E38. The method as set forth in E1-E37, wherein one or more of the individual doses are at an amount of at least 300 mg.
E39. The method as set forth in E1-E38, wherein one or more of the individual doses are at an amount of at least 350 mg, or at least 400 mg.
E40. The method as set forth in E1-E39, wherein one or more of the individual doses are at an amount of at least 450 mg.
E41. The method as set forth in E1-E40, wherein one or more of the individual doses are at an amount of at least 500 mg.
E42. The method as set forth in E1-E41, wherein one or more of the individual doses are at an amount of at least 550 mg.
E43. The method as set forth in E1-E42, wherein one or more of the individual doses are at an amount of at least 600 mg.
E44. The method as set forth in E1-E43, wherein one or more of the individual doses are at an amount of 140 mg.
E45. The method as set forth in E1-E44, wherein one or more of the individual doses are at an amount of 150 mg.
E46. The method as set forth in E1-E45, wherein one or more of the individual doses are at an amount of 200 mg.
E47. The method as set forth in E1-E46, wherein one or more of the individual doses are at an amount of 250 mg.
E48. The method as set forth in E1-E47, wherein one or more of the individual doses are at an amount of 300 mg.
E49. The method as set forth in E1-E48, wherein one or more of the individual doses are at an amount of 350 mg.
E50. The method as set forth in E1-E49, wherein one or more of the individual doses are at an amount of 450 mg.
E51. The method as set forth in E1-E50, wherein one or more of the individual doses are at an amount of 500 mg.
E52. The method as set forth in E1-E51, wherein one or more of the individual doses are at an amount of 550 mg.
E53. The method as set forth in E1-E54, wherein one or more of the individual doses are at an amount of 600 mg.
E54. The method as set forth in E1-E53, wherein one or more of the individual doses are at an amount of 900 mg.
E55. The method as set forth in E1-E54, wherein the majority of the individual doses are at the same amount.
E56. The method as set forth in E1-E53, wherein the individual doses are at the same amount.
E57. The method as set forth in any one of E1-E56, wherein one or more of the individual doses are via intravenous injection.
E58. The method as set forth in any one of E1-E57, wherein the plurality of individual doses are via intravenous injection.
E59. The method as set forth in any one of E1-E58, wherein the plurality of individual doses are via intravenous injection, and are at an amount of at least 150 mg.
E60. The method as set forth in any one of E1-E59, wherein the plurality of individual doses are via intravenous injection, and are at an amount of at least 450 mg.
E61. The method as set forth in any one of E1-E60, wherein the plurality of individual doses are via intravenous injection, and are at an amount of at least 600 mg.
E62. The method as set forth in any one of E1-E61, wherein the plurality of individual doses are via intravenous injection, and are separated from each other by at least 4 weeks.
E63. The method as set forth in any one of E1-E62, wherein the plurality of individual doses are via intravenous injection, and are at an amount of at least 150 mg, and are separated from each other by at least 4 weeks.
E64. The method as set forth in any one of E1-E63, wherein the plurality of individual doses are via intravenous injection, and are at an amount of at least 450 mg, and are separated from each other by at least 4 weeks.
E65. The method as set forth in any one of E1-E64, wherein the plurality of individual doses are via intravenous injection, and are at an amount of at least 600 mg and are separated from each other by at least 4 weeks.
E66. The method as set forth in any one of E1-E56, wherein one or more of the individual doses are via subcutaneous injection.
E67. The method as set forth in E67, wherein the plurality of individual doses are via subcutaneous injection.
E68. The method as set forth in any one of E66-E67, wherein the plurality of individual doses are via subcutaneous injection, and are at an amount of at least 150 mg.
E69. The method as set forth in any one of E66-E68, wherein the plurality of individual doses are via subcutaneous injection, and are at an amount of at least 450 mg.
E70. The method as set forth in any one of E66-E69, wherein the plurality of individual doses are via subcutaneous injection, and are at an amount of at least 600 mg.
E71. The method as set forth in any one of E66-E70, wherein the plurality of individual doses are via subcutaneous injection, and are separated from each other by at least 1 weeks.
E72. The method as set forth in any one of E66-E71, wherein the plurality of individual doses are via subcutaneous injection, and are at an amount of at least 150 mg, and are separated from each other by at least 1 week.
E73. The method as set forth in any one of E66-E72, wherein the plurality of individual doses are via subcutaneous injection, and are at an amount of at least 450 mg, and are separated from each other by at least 1 week.
E74. The method as set forth in any one of E66-E73, wherein the plurality of individual doses are via subcutaneous injection, and are at an amount of at least 600 mg and are separated from each other by at least 1 week.
E75. The method according to E1-E74, wherein the dosing regimen is continued for at least 4 weeks.
E76. The method according to E1-E75, wherein the dosing regimen is continued for at least 1 month.
E77. The method according to E1-E76, wherein the dosing regimen is continued for at least 8 weeks.
E78. The method according to E1-E77, wherein the dosing regimen is continued for at least 2 months.
E79. The method according to E1-E78, wherein the dosing regimen is continued for at least 12 weeks.
E80. The method according to E1-E79, wherein the dosing regimen is continued for at least 3 months.
E81. The method according to E1-E80, wherein the dosing regimen is continued for at least 16 weeks.
E82. The method according to E1-E81, wherein the dosing regimen is continued for at least 4 months.
E83. The method according to E1-E82, wherein the dosing regimen is continued for at least 20 weeks.
E84. The method according to E1-E83, wherein the dosing regimen is continued for at least 5 months.
E85. The method according to E1-E84, wherein the dosing regimen is continued for at least 24 weeks.
E86. The method according to E1-E85, wherein the dosing regimen is continued for at least 6 months.
E87. The method according to E1-E86, wherein the dosing regimen is continued for at least 26 weeks.
E88. The method as set forth in any one of E1-E87, wherein the improvement in signs or symptoms is characterized by a clinical response.
E89. The method as set forth in any one of E1-E88, wherein at least 4 weeks following the beginning of the dosing regimen the patient experiences an improvement in signs and symptoms of one or more selected from the group consisting of IIM, dermatomyositis, polymyositis, inclusion body myositis, juvenile dermatomyositis, SLE, Cutaneous Lupus, Psoriasis, Ulcerative Colitis, Crohn's Disease, Rheumatoid Arthritis, Atopic Dermatitis and Scleroderma, characterized by a clinical response.
E90. The method as set forth in any one of E1-E89, wherein at least 4 weeks following the beginning of the dosing regimen the patient experiences an improvement in signs and symptoms of one or more selected from the group consisting of SLE, Cutaneous Lupus, and Psoriasis, characterized by a clinical response.
E91. The method as set forth in any one of E1-E89, wherein at least 4 weeks following the beginning of the dosing regimen the patient experiences an improvement in signs and symptoms of one or more selected from the group consisting of IIM, dermatomyositis, polymyositis, inclusion body myositis, and juvenile dermatomyositis.
E92. The method as set forth in any one of E1-E91, wherein at least 4 weeks following the beginning of the dosing regimen the patient experiences an improvement in IIM, characterized by a clinical response.
E93. The method as set forth in any one of E1-E92, wherein at least 4 weeks following the beginning of the dosing regimen the patient experiences an improvement in dermatomyositis, characterized by a clinical response.
E94. The method as set forth in any one of E1-E93, wherein at least 4 weeks following the beginning of the dosing regimen the patient experiences an improvement in polymyositis, characterized by a clinical response.
E95. The method as set forth in any one of E1-E94, wherein at least 4 weeks following the beginning of the dosing regimen the patient experiences an improvement in inclusion body myositis, characterized by a clinical response.
E96. The method as set forth in any one of E1-E95, wherein at least 4 weeks following the beginning of the dosing regimen the patient experiences an improvement in juvenile dermatomyositis, characterized by a clinical response.
E97. The method as set forth in any one of E1-E76, wherein the clinical response is measured by an assessment of skin lesions.
E98. The method of E88-E97, wherein the clinical response may be characterized by a change from baseline in Manual Muscle Testing (MMT-8) score of greater than zero.
E99. The method of E88-E98, wherein the clinical response may be characterized by a change from baseline in Manual Muscle Testing (MMT-8) score of at least 5.
E100. The method of E88-E99, wherein the clinical response may be characterized by a change from baseline in Manual Muscle Testing (MMT-8) score of at least 7.
E101. The method of E88-100, wherein the clinical response may be characterized by a change from baseline in Manual Muscle Testing (MMT-8) score of at least 9.
E102. The method of E88-E101, wherein the clinical response may be characterized by a change from baseline in Manual Muscle Testing (MMT-8) score of at least 15.
E103. The method of E88-E102, wherein the clinical response may be characterized by a change from baseline in Manual Muscle Testing (MMT-8) score of at least 20.
E104. The method of E88-103, wherein the clinical response may be characterized by an improvement in Total Improvement Score (TIS) of greater than zero.
E105. The method of E88-E104, wherein the clinical response may be characterized by an improvement in Total Improvement Score (TIS) of at least 20.
E106. The method of E88-E105, wherein the clinical response may be characterized by an improvement in Total Improvement Score (TIS) of at least 25.
E107. The method of E88-E106, wherein the clinical response may be characterized by an improvement in Total Improvement Score (TIS) of at least 30.
E108. The method of E88-E107, wherein the clinical response may be characterized by an improvement in Total Improvement Score (TIS) of at least 35.
E109. The method of E88-E108, wherein the clinical response may be characterized by an improvement in Total Improvement Score (TIS) of at least 40.
E110. The method of E88-E109, wherein the clinical response may be characterized by an improvement in Total Improvement Score (TIS) of at least 45.
E111. The method of E88-E110, wherein the clinical response may be characterized by an improvement in Total Improvement Score (TIS) of at least 50.
E112. The method of E88-E111, wherein the clinical response may be characterized by an improvement in Total Improvement Score (TIS) of at least 55.
E113. The method of E88-E112, wherein the clinical response may be characterized by a change from baseline in Patient Global Assessment score of less than zero.
E114. The method of E88-E113, wherein the clinical response may be characterized by a change from baseline in Patient Global Assessment score of at least −1 on a 10-centimeter visual analog scale (VAS).
E115. The method of E88-E114, wherein the clinical response may be characterized by a change from baseline in Patient Global Assessment score of at least −2 on a 10-centimeter VAS.
E116. The method of E88-E115, wherein the clinical response may be characterized by a change from baseline in Patient Global Assessment score of at least −3 on a 10-centimeter VAS.
E117. The method of E88-E116, wherein the clinical response may be characterized by a change from baseline in Patient Global Assessment score of at least −4 on a 10-centimeter VAS.
E118. The method of E88-E117, wherein the clinical response may be characterized by a change from baseline in Patient Global Assessment score of at least −5 on a 10-centimeter VAS.
E119. The method of E88-E118, wherein the clinical response may be characterized by an improvement in absolute muscle enzyme creatinine kinase.
E120. The method of E88-E119, wherein the clinical response may be characterized by an improvement in absolute muscle enzyme creatinine kinase of at least −75 U/L.
E121. The method of E88-E120, wherein the clinical response may be characterized by an improvement in absolute muscle enzyme creatinine kinase of at least −100 U/L.
E122. The method of E88-E121, wherein the clinical response may be characterized by an improvement in absolute muscle enzyme creatinine kinase of at least −125 U/L.
E123. The method of E88-E122, wherein the clinical response may be characterized by an improvement in absolute muscle enzyme creatinine kinase of at least −150 U/L.
E124. The method of E88-E123, wherein the clinical response may be characterized by an improvement in absolute muscle enzyme creatinine kinase of at least −175 U/L.
E125. The method of E88-E124, wherein the clinical response may be characterized by an improvement in absolute muscle enzyme creatinine kinase of at least −185 U/L.
E126. The method of E88-E125, wherein the clinical response may be characterized by a change from baseline in Cutaneous Dermatomyositis Disease Area and Severity Index (CDASI-A) greater than zero.
E127. The method of E88-E126, wherein the clinical response may be characterized by a change from baseline in Cutaneous Dermatomyositis Disease Area and Severity Index (CDASI-A) of at least −5.
E128. The method of E88-E127, wherein the clinical response may be characterized by a change from baseline in Cutaneous Dermatomyositis Disease Area and Severity Index (CDASI-A) of at least −10.
E129. The method of E88-E128, wherein the clinical response may be characterized by a change from baseline in Cutaneous Dermatomyositis Disease Area and Severity Index (CDASI-A) of at least −12.
E130. The method of E88-E129, wherein the clinical response may be characterized by a change from baseline in Cutaneous Dermatomyositis Disease Area and Severity Index (CDASI-A) of at least −14.
E131. The method of E88-E130, wherein the clinical response may be characterized by a change from baseline in Cutaneous Dermatomyositis Disease Area and Severity Index (CDASI-A) of at least −20.
E132. The method as set forth in any one of E1-E131, wherein at least 8 weeks following the beginning of the dosing regimen the patient experiences an improvement in signs and symptoms that are maintained for at least a further 4 weeks.
E133. The method as set forth in any one of E1-132, wherein at least 8 weeks following the beginning of the dosing regimen the patient experiences an improvement in signs and symptoms that are maintained for at least a further 6 weeks.
E134. The method as set forth in any one of E1-E133, wherein at least 8 weeks following the beginning of the dosing regimen the patient experiences an improvement in signs and symptoms that are maintained for at least a further 8 weeks.
E135. The method as set forth in any one of E1-E134, wherein at least 8 weeks following the beginning of the dosing regimen the patient experiences an improvement in signs and symptoms that are maintained for at least a further 12 weeks.
E136. The method as set forth in any one of E1-E135, wherein at least 8 weeks following the beginning of the dosing regimen the patient experiences an improvement in signs and symptoms that are maintained for at least a further 16 weeks.
E137. The method as set forth in any one of E1-E136, wherein at least 8 weeks following the beginning of the dosing regimen the patient experiences an improvement in signs and symptoms that are maintained for at least a further 20 weeks.
E138. The method as set forth in any one of E1-E137, wherein at least 8 weeks following the beginning of the dosing regimen the patient experiences an improvement in signs and symptoms that are maintained for at least a further 24 weeks.
E139. The method according to E1-E138, wherein the improvement in signs and symptoms is experienced at least 8 weeks after beginning the dosing regimen.
E140. The method according to E1-E139, wherein the improvement in signs and symptoms is experienced at least 10 weeks after beginning the dosing regimen.
E141. The method according to E1-E140 wherein the improvement in signs and symptoms is experienced at least 12 weeks after beginning the dosing regimen.
E142. The method according to E1-E141, wherein the improvement in signs and symptoms is experienced at least 16 weeks after beginning the dosing regimen.
E143. The method according to E1-E142, wherein the improvement in signs and symptoms is experienced at least 20 weeks after beginning the dosing regimen.
E144. The method according to E1-E143, wherein the improvement in signs and symptoms is experienced at least 24 weeks after beginning the dosing regimen.
E145. The method according to any E1-E144, wherein the patient is treated concomitantly to the dosage regimen with an anti-IFNß with at least one other medication.
E146. The method according to any E1-E145, wherein the patient was previously treated with at least one other medication.
E147. The method according to E125-E146, wherein the at least one other medication is selected from the group consisting of corticosteroids, IVIG, and an immunomodulating and immunosuppressive drug.
E148. The method according to E147, wherein the immunomodulating and immunosuppressive drug is selected from the group consisting of hydroxychloroquine, azathioprine, mycophenolate mofetil, and methotrexate.
E149. The method according to E1-E148, wherein the patient shows a clinical response after 24 weeks of treatment.
E150. The method according to E1-E149, wherein the patient shows a clinical response after 20 weeks of treatment.
E151. The method according to E1-E150, wherein the patient shows a clinical response after 16 weeks of treatment.
E152. The method according to E1-E151, wherein the patient shows a clinical response after 12 weeks of treatment.
E153. The method according to E1-E152, wherein the patient shows a clinical response after 8 weeks of treatment.
E154. The method according to E1-E153, wherein the patient shows a clinical response after 4 weeks of treatment.
E155. The method according to E1-E154, wherein the anti-IFNß antibody comprises three CDRs from the variable heavy chain region having the sequence shown in SEQ ID NO: 3 and three CDRs from the variable light chain region having the sequence shown in SEQ ID NO: 4.
E156. The method according to E1-E155, wherein the anti-IFNß antibody comprises a HCDR1 having the sequence shown in SEQ ID NO: 5, a HCDR2 having the sequence shown in SEQ ID NO: 6, a HCDR3 having the sequence shown in SEQ ID NO: 7, a LCDR1 having the sequence shown in SEQ ID NO: 8, a LCDR2 having the sequence shown in SEQ ID NO: 9, and a LCDR3 having the sequence shown in SEQ ID NO:10.
E157. The method according to E1-E156, wherein the anti-IFNß antibody comprises a variable heavy chain region having the sequence shown in SEQ ID NO: 3 and a variable light chain region having the sequence shown in SEQ ID NO: 4.
E158. The method according to E1-E157, wherein the anti-IFNß antibody comprises a heavy chain having the sequence shown in SEQ ID NO: 1 and a light chain having the sequence shown in SEQ ID NO: 2, wherein the C-terminal lysine (K) of the heavy chain amino acid sequence of SEQ ID NO: 1 is optional.
E159. The method according to E1-E158, wherein the anti-IFNß antibody comprises a VH encoded by the nucleic acid sequence of the insert of the vector deposited as CTI-AF1-VH having ATCC accession number PTA-122727 and a VL encoded by the nucleic acid sequence of the insert of the vector deposited as CTI-AF1-VL having ATCC accession number PTA-122726.
E160. The method of any one of E1-E159, wherein the antibody comprises the VH sequence encoded by the insert in the plasmid deposited with the ATCC and having ATCC Accession No. PTA-122727.
E161. The method of any one of E1-E160, wherein the antibody comprises the VL sequence encoded by the insert in the plasmid deposited with the ATCC and having ATCC Accession No. PTA-122726.
E162. The method according to E1-E161, wherein the anti-IFNß antibody competes for binding with an anti-IFNß antibody comprising a variable heavy chain region having the sequence shown in SEQ ID NO: 3 and a variable light chain region having the sequence shown in SEQ ID NO: 4.
E163. The method according to E1-E162, wherein the anti-IFNß antibody competes for binding with an antibody comprising a VH encoded by the nucleic acid sequence of the insert of the vector deposited as CTI-AF1-VH having ATCC accession number PTA-122727 and a VL encoded by the nucleic acid sequence of the insert of the vector deposited as CTI-AF1-VL having ATCC accession number PTA-122726.
E164. The method according to any one of E1-E162, wherein the JDM patient is one or more of at least 12 years of age, at least 30 kg in weight, or at least 40 kg in weight.
E165. Use of an anti-IFNß antibody for the preparation of a medicament for a method of treatment according to any of E1-E164.
E166. An anti-IFNß antibody for use according to a method of any one of E1-E165.
E167. Use of an anti-IFNß antibody in the preparation of a medicament for treating a patient according to a method of any one of E1-E166.
E168. An aqueous formulation comprising:
   An anti-IFNß antibody at a concentration of between 25 mg/mL and 200 mg·mL;
   Histidine or His-HCL at a concentration of between 10 and 50 mM;
   Arginine or NaCL in an amount 20-150 mM;
   Sucrose or Trehalose in an amount between 20 mg/ml and 85 mg/ml;
   At a pH of between pH 5.0 and pH 6.5.
E169. The formulation of E168, wherein the formulation further comprises a chelator.
E170. The formulation as set forth in E169, wherein the chelator is present at an amount of between 0.01 and 0.1 mg/ml.
E171. The formulation as set forth in E170, wherein the chelator is present at an amount of between 0.02 and 0.08 mg/ml.
E172. The formulation as set forth in E171, wherein the chelator is present at an amount of 0.05 mg/ml.
E173. The formulation as set forth in E69-E172, wherein the chelator is EDTA.
E174. The formulation as set forth in E169-E173, wherein the chelator is EDTA and is present in an amount of 0.05 mg/ml.
E175. The formulation of E168-E174, wherein the formulation further comprises a surfactant.
E176. The formulation of E174-E175, wherein the surfactant is present at an amount of between 0.05 and 0.5 mg/ml.
E177. The formulation of E174-E176, wherein the surfactant is present at an amount of between 0.1 and 0.3 mg/ml.
E178. The formulation of E174-E177, wherein the surfactant is present at an amount of 0.2 mg/ml.
E179. The formulation of E174-E178, wherein the surfactant is PS80.
E180. The formulation of E174-E179, wherein the surfactant is PS80 and is present in an amount of 0.2 mg/ml.
E181. The formulation of E168-E180, wherein the Histidine or His-HCL is present at an amount of between 2 mM and 50 mM.
E182. The formulation of E168-E181, wherein the Histidine or His-HCL is present at an amount of between 5 mM and 30 mM.
E183. The formulation of E168-E182, wherein the Histidine or His-HCL is present at an amount of between 10 mM and 30 mM.
E184. The formulation of E168-E183, wherein the Histidine or His-HCL is present at an amount of 20 mM. E185. The formulation of E168-E184, wherein the formulation comprises Histidine.
E186. The formulation of E168-E185, wherein the formulation comprises Histidine at an amount of 20 mM.
E187. The formulation of E168-E186, wherein the Arginine or NaCl is present at an amount of between 50 mM and 150 mM.
E188. The formulation of E168-E187, wherein the Arginine or NaCl is present at an amount of between 50 mM and 100 mM.
E189. The formulation of E168-E188, wherein the Arginine or NaCl is present at an amount of 50 mM.
E190. The formulation of E168-E189, wherein the formulation comprises Arginine.
E191. The formulation of E168-E190, wherein the formulation comprises Arginine at an amount of 50 mM.
E192. The formulation of E168-E191, wherein the sucrose or trehalose is present at an amount of between 25 and 75 mg·ml.
E193. The formulation of E168-E192, wherein the sucrose or trehalose is present at an amount of between 50 and 75 mg·ml.
E194. The formulation of E168-E193, wherein the sucrose or trehalose is present at an amount of 50 mg·ml.
E195. The formulation of E168-E194, wherein the formulation comprises sucrose.
E196. The formulation of E168-E195, wherein the formulation comprises sucrose at an amount of 50 mg/ml.
E197. The formulation of E168-E196, wherein the pH is between 5.5 and 6.0.
E198. The formulation of E168-E197, wherein the pH is 5.8.
E199. The formulation of E168-E198, wherein the anti-IFNß antibody is present at an amount of at least 50 mg·ml.
E200. The formulation of E168-E199, wherein the anti-IFNß antibody is present at an amount of at least 60 mg·ml.
E201. The formulation of E168-E200, wherein the anti-IFNß antibody is present at an amount of 60 mg·ml.
E202. The formulation of E168-E201, wherein the anti-IFNß antibody is present at an amount of at least 80 mg·ml.
E203. The formulation of E168-E202, wherein the anti-IFNß antibody is present at an amount of at least 100 mg·ml. or at least 120 mg·mL.
E204. The formulation of E168-E203, wherein the anti-IFNß antibody is present at an amount of between about 141 and 154 mg·mL.
E205. The formulation of E168-E204, wherein the anti-IFNß antibody is present at an amount of at least 150 mg·ml.
E206. The formulation of E168-E205, wherein the anti-IFNß antibody is present at an amount of 150 mg·ml.
E207. The formulation of E168-E206, wherein the anti-IFNß antibody is as described in any one of E155-E163.
E208. The formulation of E168-E207, wherein the formulation comprises
   60 mg/mL anti IFNß as described in any one of E155-E163;
   20 mM histidine;
   50 mg/mL sucrose;
   50 mM Arginine;
   0.05 mg/mL EDTA;
   0.2 mg/mL polysorbate 80; and
   pH 5.8.
E209. The formulation of E168-E207, wherein the formulation comprises
   150 mg/mL anti IFNß as described in any one of E155-E163;
   20 mM histidine;
   50 mg/mL sucrose;
   50 mM Arginine;
   0.05 mg/mL EDTA;
   0.2 mg/mL polysorbate 80; and
   pH 5.8.
E210. The formulation of E168-E207, wherein the formulation comprises
   Between 141 and 154 mg/mL anti IFNß as described in any one of E155-E163;
   20 mM histidine;
   50 mg/mL sucrose;
   50 mM Arginine;
   0.05 mg/mL EDTA;
   0.2 mg/mL polysorbate 80; and
   pH 5.8.
E211. The formulation of any one of E168-E210, wherein the formulation has a viscosity of less than 20 centipoles.
E212. The formulation of any one of E168-E211, wherein the formulation has a viscosity of less than 15 centipoles.
E212. The formulation of any one of E168-E212, wherein the formulation has an osmolality of less than 500 mOsm.
E213. The formulation of any one of E168-E213, wherein the formulation has an osmolality of less than 400 mOsm.
E214. The formulation of any one of E168-E214, for use in the method of any one of E1-E143, or the use of E144 or E146,
E215. The method as set forth in any one of E1-E164, wherein the antibody is formulated in the formulation as set forth in any one of E168-E214.
E216. The method as set forth in any one of E1-E164, comprising administering to the subject a therapeutically effective amount of the aqueous formulation of any one of E168-E214.

### BRIEF DESCRIPTION OF THE FIGURES/DRAWINGS

FIG. 1 Stage 1 (with planned sample sizes). Participants with skin involvement (CDASI-Activity ≥14 at screening) and have failed at least 1 standard of care systemic treatment, (eg, corticosteroids) were randomized to receive 600 mg of PF-06823859 or placebo in a 2:1 ratio. Investigational drug or placebo administration took place on Day 1, Week 4, and Week 8. The primary endpoint (CFB CDASI-A) was assessed at Week 12.

FIG. 2 Stage 2 (with planned sample sizes). Participants with skin involvement (CDASI-Activity 14 at screening) were randomized to receive 600 mg of PF-06823859, 150 mg of PF-06823859, or placebo in a 5:11:4 ratio. Investigational drug or placebo administration took place on Day 1, Week 4, and Week 8 of the study. The primary endpoint (CFB CDASI-A) was assessed at Week 12.

FIG. 3 Amended Stage 2 (with Planned Sample Sizes). A fixed sequence design was employed in Amended Stage 2 to provide all study participants with the opportunity to receive active drug during the treatment period. Participants were randomized to one of the following sequences in a 5:11:2:2 ratio: 600 mg PF-06823859 then placebo, 150 mg PF-06823859 then placebo, placebo then 600 mg PF-06823859, or placebo then 150 mg PF-06823859. Investigational drug or placebo administration (as dictated by the treatment sequence) occurred on Day 1, Week 4, Week 8, Week 12, Week 16, and Week 20. The primary endpoint (CFB CDASI-A) was assessed at Week 12 of Amended Stage 2. After the treatment period ended at Week 24, participants then entered a 4-month follow-up period or rolled over to the long-term extension study, C0251008.

FIG. 4 Stage 3 (with Planned Sample Sizes). A fixed sequence design was also employed in Stage 3 where participants with predominantly muscle involvement were randomized to one of the following sequences in a 1:1 ratio: 600 mg PF-06823859 then placebo, or placebo then 600 mg PF-06823859 with a treatment switch at Week 12. The inclusion criteria for the muscle involvement required that the subject met one of the following two criteria: (1) MMT-8≤136/150 and PhGA (VAS ≥3 cm on 0-10 cm scale) or (2) sum of PhGA, PtGA, and extramuscular global assessment is ≥10 cm (using 0-10 cm VAS scale for each) and had failed at least two or more adequate courses of an immunosuppressive or immunomodulatory agent, including IVIG. Immunosuppressive and immunomodulatory agents including Intravenous Immunoglobulin (IVIG) in stable doses were allowed as concomitant medications. Investigational drug or placebo administration (as dictated by the treatment sequence) occurred on Day 1, Week 4, Week 8, Week 12, Week 16, and Week 20. The secondary endpoint (TIS) was assessed longitudinally at weeks 4, 8 and 12 (Week 12 being the key timepoint). Other secondary muscle-related endpoints are: MMT-8 and PtGA of Myositis. After Week 12 they were switched to the other treatment in the sequence. After the treatment period ended at Week 24, participants entered a 4-month follow-up period or rolled over to the long-term extension study, C0251008.

FIG. 5 Mean Absolute Values and Difference from Placebo in TIS (MMRM, Week 4 to Week 12).

FIG. 6 Mean Changes from Baseline for MMT-8 (LANCOVA, Week 4 to Week 12).

FIG. 7 Mean Changes from Baseline for Patient Global assessment of Myositis (LANCOVA), where the range of Patient Global Assessment is 0 to 100 using the 10 cm (i.e., 100 mm) VAS.

FIG. 8 Mean Changes from Baseline for CK (U/L) (LANCOVA, Week 1 to Week 12).

FIG. 9 Mean Estimated CFB of CDASI Activity Scores (LANCOVA-P, Baseline— Week 12, FAS1 & Pooled FAS for Skin Cohort (PFASS, which includes the first 12 weeks of data from subjects in Stage 1, Stage 2, and Amended Stage 2).

FIG. 10. Strength and significance of the 10-gene type-1 IFN signature across relevant diseases highlighting additional opportunities beyond Dermatomyositis (“DM”).

FIG. 11. Contextualizing the effect of Pfizer's anti-IFNß molecule in the dermatomyositis trial split by dose arm. Patients in each dose arm have comparable type-1 IFN signatures at baseline (Baseline: circles). After treatment, the type-1 IFN signature in placebo treated patients is slightly increased whereas the signature in both dose arms of the anti-IFNß molecule is ablated (Treated: triangles).

FIG. 12A: pH effect on anti-IFNß viscosity at different concentrations. FIG. 12B: formulation effect on anti-IFNß viscosity at different concentrations.

FIG. 13. Effect of Arginine Concentration on anti-IFNß Viscosity.

FIG. 14. Effect of Sodium Chloride on anti-IFNß Viscosity

FIG. 15. Visual Predictive Check for the Final Pharmacokinetic Mode. Speckled represents the median at each binned timepoint, with 5th and 95th percentiles in hatched. Solid lines are for observed, ribbons are the 90% distribution in simulations and dashed lines are the simulation medians.

FIG. 16. Visual Predictive Check for IFNß in the Final PKPD Model. Speckled represents the median at each binned timepoint, with 5th and 95th percentiles in hatched. Solid lines are for observed, ribbons are the 90% distribution in simulations and dashed lines are the simulation medians. Observations are black points.

FIG. 17. Simulations of Pharmacokinetics of Doses Followed for 52 Weeks. PF-06823859 median plasma concentrations are in solid black with 90% prediction intervals for that median in dashed line. The predicted drug-IFNß complex concentration is in gray below, with units pg/mL. The KSS is shown as a dotted grey line is units of ng/mL.

FIG. 18. Simulations of Biomarkers Following Doses Over 52 Weeks. All solid lines represent the median value and the dashed lines are the 90% prediction interval for that median. For IFNβ, a dashed horizontal line shows the LLOQ, and dotted lines indicate the median bounds for the simulated 90% distribution of IFNβ.

FIG. 19(A), FIG. 19(B), FIG. 19(C), FIG. 19(D), FIG. 19(E), FIG. 19(F). Dose and Frequency Options and Simulated Summary Biomarker Responses. For FIG. 19(A), FIG. 19(B), and FIG. 19(C), frequency of doses (when more than 1 was given) is fixed to every 4 weeks. For FIG. 19(D), FIG. 19(E), and FIG. 19(F), number of doses is fixed to 3. Points represent the median endpoint, and error bars represent the 90% predictions intervals of the medians. Lines are just connecting points, and are neither smoothed nor imputational.

FIG. 20. Simulated Percent of Adolescent Subjects with Exposure Ratios Greater than 2 for Various Weight-Based Dosing Thresholds. At each cutoff, subjects below the index weight are given the 9 mg/kg dose, and subjects at or above are given the 600 mg dose. The median (black, solid) percent of subjects at weight-based dosing cutoffs between 30 and 70 kg and the 90% prediction interval of that median (grey ribbon) are shown. A vertical solid line and corresponding label indicate where the median intersects with 10%, and the 90% prediction interval indicates where the upper and lower estimates intersect with 10%.

FIG. 21. PK simulations to Match Trough Between SC dosing Regimens vs. the Reference IV Dose. X-axis represents time post dose in weeks, Y-axis represents the geometric mean concentration (ng/mL). The dosing regimens are dotted (600 mg IV Q4W), dash-dot (750 mg SC Q4W), dash (300 mg SC Q2W), and fine-dot (150 mg SC QW). Abbreviations: IV, intravenous; ml, milliliter; ng, nanogram; QW, weekly; Q2W, every 2 weeks; Q4W, every 4 weeks; SC, subcutaneous.

FIG. 22. PK simulations to Match AUC/Cave Between SC dosing Regimens vs. the Reference IV Dose. X-axis represents time post dose in weeks, Y-axis represents the geometric mean concentration (ng/mL). The dosing regimens are in dash-dot (600 mg IV Q4W), dotted (900 mg SC Q4W), and dashed (225 mg SC QW). Abbreviations: IV, intravenous; ml, milliliter; ng, nanogram; QW, weekly; Q4W, every 4 weeks; SC, subcutaneous.

### DETAILED DESCRIPTION OF THE INVENTION

Data for the Stage 2 (dose ranging in skin predominant DM) and the placebo-controlled 12 weeks of Stage 3 (muscle predominant DM) pointed to a surprisingly high level of efficacy. In particular, the data from the muscle cohort of the Phase 2 study demonstrated efficacy supporting the efficacy data on skin manifestations. Therefore, the study demonstrates the potential for an IFNß neutralizing antibody to offer an effective treatment for DM/JDM and PM, and modelling data provided herein supports efficacy of comparable doses to other indications, in particular SLE, cutaneous lupus, and psoriasis.

DM is characterized by B-cell activation and autobody-mediated inflammation and tissue damage. Scientific literature in DM supports the hypothesis that IFNß protein levels are elevated in DM blood and mRNA levels are increased in DM skin. Capelletti et al, showed that multiple components of the IFN α/β-mediated responses are upregulated in muscle samples of myositis patients compared to controls (Capelletti et al, 2011). The most expressed in their analysis were ISG15, IFIT3, MX1 and IFIT1 which are regulators of biological and therapeutic effects of IFNα and IFNβ. In this study, IFN mediated molecules were expressed in JDM, DM and PM, with the highest observed in samples from JDM patients. Lastly, IFNβ mRNA was upregulated, pointing to this IFN as the one likely responsible for the upregulation of the mentioned genes (Baechler et al. 2011).

Type 1 IFN inducible genes are elevated in blood and muscle from patients with PM, albeit to a lesser extent than seen in DM. Detection of IFNβ in PM samples is more limited but may also be hampered by current assay sensitivity. When the expression levels of IFN in the peripheral blood and muscle of patients with IIM were analyzed to determine their relationships with organ manifestations, the expression levels of IFNα and IFNβ in PM/DM patients are significantly higher than those in normal controls and the increments had correlations with disease activity and severity (Zu & Wang, 2011).

DM and JDM are sufficiently similar in terms of clinical presentation, pathophysiology and histopathology for these to be assessed as a combined cohort for efficacy, cognizant of the rarity of DM and JDM.

As reported in the literature, improvement in muscle function when receiving a therapy that is deemed efficacious, is observed within the first 6 months of treatment. An example of this was described by Ruperto et al, (Ruperto et al, 2016) who showed that >50% of JDM patients treated with either prednisone alone or prednisone in combination with methotrexate or cyclosporine, achieved a ≥20% improvement in 3 of the 6 CSMs after 6 months of therapy. Similarly, the Proderm study showed that 78.7% of DM patients treated with Octagam achieved a minimal improvement in TIS after 16 weeks of treatment

Given the early onset of efficacy for skin manifestations in participants receiving PF-06823859 in present study (plateauing by Week 12), and the clear demonstration of efficacy on muscle function by Week 12, an evaluation at 24 weeks (moderate improvement in TIS) will enable the observation of the maximum treatment effect of PF-06823859 in muscle manifestations as well as skin manifestations of disease. An exposure-response model was developed linking IFNb inhibition to various clinical endpoints (including CDASI, SF-36 and TIS component scores); the model predicted strong TIS response which would begin to plateau around 12 weeks and hence an endpoint at 24 weeks allows for consideration of possible waning with relatively short term treatment and, conservatively, for the potential for slower attainment of maximum efficacy in PM patients.

PM and DM patients share the cardinal feature of muscle weakness. Further, the Type 1 IFN inducible genes are elevated in blood and muscle from patients with PM. Given the understanding of the high level of target engagement and demonstrated clinical efficacy for 600 mg Q4W dose in DM patients, the same dose is expected to achieve a similar level of efficacy in PM patients.

The TIS is a weighted measure of improvement from baseline in 6 Core Set Measures (CSMs): 1) MDGA-VAS {Physician Global Assessment VAS; equivalently, PhGA-VAS}; 2) PtGA-VAS (Patent Global Assessment VAS); 3) MMT-8 (Manual Muscle Testing-8); 4) Muscle enzymes; 5) MDAAT (Myositis Disease Activity Assessment Tool); and 6) HAQ-DI (Health Assessment Question Disability Index; IMACS; Aggarwal et al, 2017). The IMACS developed a consensus on a set of core domains and measures for the assessment of disease activity in patients with DM, JDM and PM. The CSMs are accepted by ACR/EULAR and are recommended as endpoints for use in clinical trials to evaluate improvements in muscle manifestations in DM, JDM and PM. This criteria set has been approved by the ACR Board of Directors and the EULAR Executive Committee, which signifies that the criteria set has been quantitatively validated using patient data, and it has undergone validation based on an independent data set (Aggarwal et al, 2017). Also, TIS as a CSM was assessed by the PRINTO group and demonstrated utility in pediatric patients (Ruperto & Martini, 2011). Of note, in the context of TIS, as recommended by ACR/EULAR/PRINTO, physical function in pediatrics including adolescents is measured via the CHAQ-Disability Index, which assesses disease impact on activity at school, and was derived from the adult HAQ-Disability Index, which in turns assesses disease impact on activity at work.

The TIS is the sum of the improvement reflected in each of the 6 CSMs (Table 1), but the individual CSMs are weighted, such that those deemed more important provide a greater contribution to the final score. For instance, changes in the MMT scores are weighted more heavily than changes in the most abnormal enzyme or HAQ (Aggarwal et al, 2017).

TABLE 1
Core Set Measures of TIS in DM, JDM and PM

| | | | |
|---|---|---|---|
| Physician Global | ClinRO | Disease | Partially validated tool |
| Activity | | activity-overall | measures overall |
| VAS (From | | rating (based | disease activity of the |
| MDAAT*) | | on clinical and | patient by physician at |
| | | lab results) | the time of |
| | | | assessment using a |
| | | | 10 cm VAS. <1 min |
| | | | to complete. |
| | | | Reliability of serial |
| | | | ratings dependent on |
| | | | examining previous |
| | | | scores. |
| | | | Included in MDAAT |
| Patient Global | PRO | Disease | Partially validated |
| Activity VAS | | activity-overall | tool for overall |
| | | rating | disease activity as |
| | | | reported by the |
| | | | patient (or parent if |
| | | | pt. is minor) at the |
| | | | time of assessment |
| | | | using a 10 cm VAS. <1 |
| | | | min to complete. |
| MMT-8 | PerfO | Measure of | Partially validated |
| | | muscle strength | tool assessed muscle |
| | | of 8 key | strength and is |
| | | muscle groups | recommended as a |
| | | | CSM. MMT-8 is an |
| | | | abbreviated group of |
| | | | 8 proximal, distal, |
| | | | and axial muscles. <5 |
| | | | min to assess 8 |
| | | | key muscle groups |
| HAQ - Disability | PRO | Includes 20 | Recommended tool |
| Index (Bruce & | | items in 8 | in adults to assess |
| Fries, 2005) | | sections | physical function as a |
| (CHAQ - | | assessing | CSM. |
| Disability Index | | activities of | HAQ-DI and Pain |
| for juvenile | | daily living | Scale can be |
| patients) | | | completed in ~5 |
| | | | minutes. The full |
| | | | HAQ takes ~20-30 |
| | | | min to complete. |
| MDAAT - Extra | ClinRO | Extra muscular | Validated tool to |
| muscular Global | | activity (6 extra | measure disease |
| Assessment | | muscular | activity of extra- |
| | | organs) | muscular organ |
| | | | systems and muscle. |
| | | | 15-30 min to |
| | | | complete |
| Enzyme (most | Biomarker | Enzyme | Partially validated |
| abnormal) | | | tool to measure |
| | | | serum activities of at |
| | | | least 2 of the 4 |
| | | | muscle-associated |
| | | | enzymes including |
| | | | CK, ALT, AST, LDH |
| | | | and aldolase. |
| | | | Administration time: |
| | | | NA |

*Global Disease Activity VAS from the MDAAT Instrument.

Considering the key clinical manifestations of DM, JDM and PM in muscle and significant impairment in patient's HRQoL, and in view of the validation of the CSM described above, TIS is a valid assessment to evaluate the effects of treatment with PF-06823859 on the muscle manifestations of DM, JDM and PM.

### Rationale for Use of TIS Moderate Improvement as the Primary Endpoint

The TIS uses a scale of 0-100 to provide a quantitative assessment of degree of response to a therapeutic intervention for each patient. The thresholds for TIS improvement categories are tabulated in Table 2 below.

TABLE 2
Thresholds for TIS Improvement Categories

| | | Total |
|---|---|---|
| | Improvement | Improvement |
| | Category | Score |
| DM/PM | Minimal | ≥20 |
| Threshold | Moderate | ≥40 |
| | Major | ≥60 |
| JDM | Minimal | ≥30 |
| Threshold | Moderate | ≥45 |
| | Major | ≥70 |

The categorical outcomes of response were validated as part of the 2016 ACR/EULAR myositis response criteria for adult DM/PM and JDM. Rider L. et al, described the myositis response criteria and pointed that although the validation was conducted with limited data, it had some important advantages, including the possibility to use the same definition of improvement (minimal, moderate, major) across the DM, PM and JDM subtypes (Rider et al 2018). Other advantages include the fact that it does not require a minimal severity level in myositis at baseline, and all levels of improvement in the CSM contribute to the response.

### CDASI-A

The CDASI is designed to capture the extent of cutaneous disease and was developed for use in clinical trials and longitudinal patient assessment (Anyanwu, et al, 2015. British Journal of Dermatology, 173, pp 969-974, herein incorporated by reference in entirety). Disease involvement is measured in 15 distinct anatomical locations and is rated using: a) three activity measures (erythema, scale and erosion/ulceration), b) two damage measures (poikiloderma and calcinosis), and c) presence and severity of Gottron's papules on hands, periungual changes and alopecia. The resulting activity and damage scores range from 0 to 100 and 0 to 32, respectively, where higher scores indicate greater disease severity.

The CDASI was developed with the intention of creating a valid and reliable measure of skin involvement in DM and has been shown to be an effective outcome instrument in clinical trials. A separate study in 103 patients with DM (Ahmed et al, 2020a; Ahmed et al, 2020b) assessed the percentage change and actual change in CDASI-A needed to achieve a meaningful improvement in QoL. The study concluded that, in patients with a threshold CDASI-A range of >14 points, a 40% change in the CDASI-A between the first 2 visits represents meaningful change in QoL (Ahmed et al, 2020a; Ahmed et al, 2020b). In addition, in interviews conducted to gather information on content validity, three (n=3) clinicians reported they would want between a 6-10-point reduction in a 12-week clinical trial; an additional two (n=2) clinicians reported they would want between 5-8-point reduction in a 24-week clinical trial. A prior study has evaluated the reliability of the CDASI in DM and JDM and confirmed the reliability of the assessment tool (Tiao et al, 2017). The development and validation work on CDASI included the characterization of disease severity and responsiveness of the instrument to clinically meaningful changes. The analysis showed similar values for the characterization of disease severity and clinical response. The authors concluded that the CDASI activity score of 19 or less characterizes mild disease, with the cut-off being somewhere between 14 and 19. In a 2017 prospective assessment of n=42 patients in DM and n=25 healthy participants, Type I IFN pathway signature biomarker in blood was found to be highly correlated with CDASI activity scores in DM and the correlation of serum IFNb with both a gene signature and CDASI suggests that IFNb drives disease activity in DM and can be a promising surrogate endpoint in clinical trials (Huard et al, 2017). Accordingly, an improvement of at least 4 CDASI units can represent significant clinical benefit.

### MMT-8

The MMT-8 is a performance outcome assessment that is an objective evaluation of muscle strength. Whilst MMT-8 is a CSM of TIS, as muscle function is among the main factors that affects the lives of patients with DM, JDM and PM. The MMT-8 is included as a IMACS CSM in treatment guidelines for DM and PM (Rider et al, 2010, herein incorporated by reference in entirety) and as a CSM per IMACS and PRINTO for JDM (Rider et al, 2018, herein incorporated by reference in entirety). It is widely used to measure muscle strength in 8 proximal, distal and axial muscle groups. Additionally, muscle strength testing, as assessed by MMT-8, is also part of the response criteria for DM, PM (Rider et al, 2010) and JDM (Rider et al, 2018).

MMT-8 has been the method of choice for assessing muscle strength in >90% of IIM clinical trials, as it is feasible, inexpensive, easily performed, requires no equipment and has adequate inter-rater and intra-rater reliability and validity when performed by a trained examiner (Rider et al, 2007; Saygin & Oddis, 2022).

### IIM

The IIM are a diverse group of autoimmune diseases characterized by chronic muscle inflammation and associated weakness. The IIM are complex, systemic diseases, with skeletal muscle involvement and frequent manifestations in other organ systems, including skin, joints, cardiopulmonary, gastrointestinal, and constitutional systems. As described above, interactions between genetic and environmental factors are thought to result in the development of the different phenotypes of IIM.

The current paradigm describing the pathophysiology of DM appears to result from an autoimmune attack on affected organs that may be triggered by environmental factors such as UV exposure, drugs, infection, and lifestyle decisions in genetically susceptible individuals (Bogdanov et al, 2018). Early pathogenic events are thought to occur in the endothelium of the endomysial blood vessels (Bogdanov et al, 2018). C3b and C4b fragments are formed when antibodies or other components activate C3, leading to the formation of C3bNEO as well as the membrane attack complex. These are then deposited in the endomysial vasculature and result in inflammation and infarction of the microvasculature, leading to muscle atrophy. Although the pathophysiology of the skin lesions is not fully understood, the same mechanisms have been proposed (Bogdanov et al, 2018). A number of environmental factors, including UV exposure, drugs, infection, and lifestyle, may play a role in the pathophysiology of this disease. Associations with HLA DRB1*0301 and DQA1*0501 in Caucasians and HLA-B7 in Asians have been reported, which suggests that there may be a genetic predisposition in DM. One of the leading hypotheses is that there appears to be a pathogenic overproduction of the IFNβ message and protein in the blood, muscle and skin of DM patients leading to damage of these tissues.

Given that the group commonly classified as PM encompasses patients that, based on MSAs, can be further sub-grouped as ASyS, IMNM and purely PM patients with no specific autoantibodies; the pathophysiology may vary among these subgroups. The pathophysiology of all of these is not completely understood. In patients with PM and MSAs associated with ASyS, it has been observed that there is an adaptive immune response driven by B cell responses in myositis, this was then linked to the presence of anti-Jo1 auto-antibodies in these patients. Subsequent findings suggest that anti-Jo-1 autoantibodies bound common auto epitomes that change titers with disease activity, linking the immune response with the clinical manifestations of myositis. Patients in this group have also been found to have autoantibodies recognizing a reductase (HMGCR) or the SRP. The HLA class II DRB1*11:01 is present in >70% of patients with IMNM suggesting this as a very strong risk factor for developing autoimmune disease. Muscle biopsies of these patients often include areas of perifascicular necrosis (more than in DM) and endomysial infiltration by T cells. Muscle tissue from patients with positive autoantibodies for HMGCD or SRP are histologically very similar. It has been observed that high titers of these autoantibodies correlate with elevation of CK which is released into the bloodstream when muscle fibers are being damaged (Lundberg et al, 2021).

Classification criteria have been developed and used to identify uniform and comparable groups of patients. In 1975, Bohan and Peter laid the foundation by providing the first set of classification criteria for IIM that divided IIMs into 5 groups. Since then, multiple classification and/or diagnostic criteria have been developed but these are not fully validated, and classification criteria have continued to evolve. In 2017, a validated classification criteria was approved by ACR and EULAR and published by (Lundberg et al, 2017). The ACR/EULAR classification criteria are validated for adult and juvenile IIM. It defines a minimum essential, easily available clinical and laboratory features to identify patients with IIM and distinguish them with high sensitivity and specificity from those with non-IIM conditions. In addition, it categorizes IIMs in major subgroups. These criteria were developed using a cohort of 976 IIM patient cases and 624 non-IIM patient cases from 47 centers across the world. Variables were compiled from published criteria and expert opinion by consensus. The ACR/EULAR criteria classifies patients as having “definite”, “probable”, and “possible” disease based on a score and corresponding probability of disease (≥50-<55%=possible, 255%-<90%=probable, ≥90%=Definite). Subclassification of IIM distinguished between adult and juvenile myositis, polymyositis, dermatomyositis, amyopathic DM or Inclusion Body Myositis.

### Overview

PF-06823859 is a potent, selective, humanized IgG1 neutralizing antibody directed against the human soluble cytokine IFNβ, a member of the type I IFN family of cytokines.

In a widely accepted model of type I IFN production (Noppert et al, 2007), stimulation of select pattern-recognition receptors (eg: TLRs; DExD/H box RNA helicases such as RIG-1 and MDA5; cGAS which activates STING; etc) cause dimerization and activation of the IFN regulatory factor IRF3, which then translocates to the nucleus and activates transcription at the IFNß (and IFNα4) promoter. IFNß protein then signals in an autocrine and paracrine manner via binding to IFNAR. This induces intracellular signaling events downstream of IFNAR that culminate in the expression of IRF7. IRF7 is required, in turn, for the transcription of multiple IFNα subtypes. Thus, in this model production of IFNß initiates IFNAR signaling and the concomitant production of IFNα. The initiating TLR activations can arise from exposure to microbe-derived PAMPs, including microbial nucleic acids, lipids, proteins, lipoproteins, etc. However, there is increasing evidence that TLRs can be similarly stimulated by endogenous self-components that are liberated during disease processes (often termed damage-associated molecular patterns, or DAMPs). It is worth noting that IRF7 expression is constitutive in pDCs, and that in pDCs TLR activations lead directly to abundant expression of IFN isoforms.

The disclosure provides result of a phase 2 multi-stage study to assess the efficacy and safety of PF-06823859 compared to placebo after 12 weeks of treatment in patients with skin disease predominant (Stage 1, Stage 2, and Amended Stage 2) or muscle disease predominant (Stage 3) dermatomyositis (DM). For 18 participants with muscle disease predominant DM, the key efficacy endpoint of interest, mean total improvement score (TIS) showed numerical advantage of PF-06823859 600 mg compared to placebo with increasing trends overtime (4-12 weeks) and no plateau by Week 12. The estimated mean (90% confidence interval (CI)) TIS at week 12 for PF-06823859 600 mg and placebo were 56.4 (41.4, 71.4) and 36.9 (22.0, 52.0), respectively, with a placebo-adjusted difference of 19.4 (−1.8, 40.7). The other endpoints related to the muscle involvement such as, mean change from baseline (CFB) in Manual Muscle Testing (MMT-8), mean CFB in Patient Global Assessment of myositis (PtGA) and mean CFB in muscle enzyme Creatine Kinase (CK) also showed numerical advantage of PF-06823859 600 mg compared to placebo at Week 12.

The estimated mean CFB (90% CI) for the muscle endpoints at Week 12 were:
1. MMT-8: 21.2 (11.9, 30.6) for PF-06823859 600 mg and 11.7 (2.3, 21.0) for placebo with a placebo-adjusted difference of 9.6 (−3.8, 23.0).
2. PtGA (cm): −4.6 (−5.9, −3.4) for PF-06823859 600 mg and −1.2 (−2.4, 0.1) for placebo, with a placebo-adjusted difference of −3.5 (−5.3, −1.6).
3. CK(U/L): −185.8 (−273.9, −97.6) for PF-06823859 600 mg and −39.9 (−125.7, 46.0) for placebo, with a placebo-adjusted difference of −145.9 (−269.4, −22.4).
   For participants with skin disease predominant DM (including participants from Stage 1 [n=32], Stage 2 [n=9], and Amended Stage 2 [n=16]), the key efficacy endpoint of interest was the Cutaneous Dermatomyositis Disease Area and Severity Index (CDASI-A). The POC criteria for Stage 1 was: (i) target value (TV) or treatment effect lower than −5, (ii) upper limit of 2 sided 90% CI for the treatment effect <0.
   The estimated CFB (90% CI) in CDASI activity scores (CDASI-A) at Week 12 were:
600 mg PF-06823859: −19.2 (−21.5, −16.8) and placebo-adjusted difference of −16.2 (−20.4, −12.1);
150 mg PF-06823859: −16.6 (−19.8, −13.4) and placebo-adjusted difference of −13.7 (−18.3, −9.0);
Placebo: −2.9 (−6.3, 0.45).

The skin cohort data shows PF-06823859 600 mg and 150 mg differentiated from placebo (p<0.0001 for both doses) with similar treatment effects between the doses. For patients with CDASI-A score >14 points, a 40% change in the CDASI-A score indicates a meaningful change in Quality of Life (Ahmed et al, 2020) and PF-06823859 achieved this decrease in CDASI-A score in more than 80% of the subjects.

Overall, both doses of PF-06823859 (600 mg and 150 mg) met the primary efficacy endpoint (exceeded the target value) for the skin cohort as assessed by CDASI-A score. Although the study was not powered for efficacy evaluation for the muscle cohort, PF-06823859 600 mg has numerically better efficacy scores than placebo across all key muscle function endpoints (TIS, MMT-8, PtGA of myositis and CK).

### Safety

There were 2 treatment discontinuations due to adverse events, both in the 600 mg group (one subject with Colitis, microscopic in Stage 1 and one subject with Leukopenia, Cytopenia and Hepatic enzyme increased in Stage 3).

TEAEs were numerically higher in the active groups compared to placebo groups in all stages and were generally mild in severity. There was no dose-dependent relationship in TEAEs observed in Stage 2 and amended Stage 2. The most common TEAEs by SOC in the study were: Infections and Infestations, Investigations, and Nervous System Disorders. There were 3 mild cases of infection in PF-06823859 groups that were treatment related compared to 2 cases in the placebo groups across all stages. There were 4 SARS-CoV-2 test positive cases in the placebo groups and 1 suspected COVID-19 case in PF-06823859 600 mg group. There were no cases of Herpes Zoster or Herpes Simplex. There were 5 participants who reported 7 SAEs, 3 in Stage 1) and 4 in Stage 3 (Humerus Fracture (600 mg group), and 3 SAEs (Leukopenia; Cytopenia and Hepatic enzyme increase) in one subject (600 mg group). There were no deaths or reports of serious adverse reactions (SARs). Also, there were no clinically significant changes from baseline in safety labs, ECG, and vital signs after treatment with PF-06823859.

Overall, 150 mg and 600 mg PF-06823859 administered once monthly for a total of 3 doses were generally well tolerated and safe in all stages of the study.

### Pharmacokinetics

PF-06823859 IV PK is consistent across disease states and healthy participants. There was an approximate dose proportional increase in in exposure over the dose range 150 mg Q4W to 600 mg Q4W in patients with predominantly skin disease. A mechanistic biomarker for IFNb inhibition, IP-10 decreased over time with comparable mean percent reduction at Week 12 in skin predominant DM patients receiving either PF-06823859 150 mg or 600 mg. The exposure response relationship appeared to be saturated by 150 mg. The overall low incidence of drug induced ADAs (3/22; Stage 1-600 mg) implies lack of clinically relevant impact of clinically relevant impact on safety/PK/PD or efficacy. There were no events of anaphylaxis or immunologically related clinical responses of concern observed. A clinical trial simulation supported PF-06823859 achieving pre-specified target product profile efficacy criteria at 24 weeks for TIS, MMT8 and CDASI-A.

In some aspect, and without wishing to be bound by theory, it is anticipated that PM patients may rely on the dosing regimen for DM patients, as proximal muscle weakness is the cardinal feature of both DM and PM, and the observation that Type 1 IFN inducible genes are elevated in blood and muscle from patients with PM (albeit to a lesser extent than in DM).

Similarly, the similarities in clinical manifestations and IFN gene expression between the JDM population and the adult DM population suggest the dosing regimens for adults will be safe and efficacious and safe in adolescents. In some embodiments, the adolescents are at least 12 years of age. In some embodiments, the adolescents weigh at least 30 kg. some embodiments, the adolescents weigh at least 40 kg.

Modeling of PK, PD, and efficacy in DM participants supports selection of the 600 mg Q4W.

Inhibition of IP-10 was high at both 150 mg and 600 mg Q4W doses, and a small increase in % inhibition of IP-10 was predicted between the 150 mg and 600 mg doses. IP-10 is a biomarker downstream of IFNß inhibition that is correlated with inflammation.

While near complete target engagement was shown at the lower dose 150 mg Q4W in the skin-predominant cohort in terms of CDASI-A clinical efficacy, the same dose was not studied in the muscle-predominant cohort. The muscle efficacy endpoints cannot be assumed based on skin efficacy endpoints due to limited understanding of correlation between 2 cohorts.

IFNβ is highly bound (>99%) to PF-06823859 at the exposure corresponding to the lower dose of 150 mg Q4W (evaluated in skin predominant DM in Stage 2), indicating near complete target engagement at 150 mg Q4W and higher doses. This is consistent with the observed clinical efficacy response for skin predominant disease, in terms of reduction in CDASI-A score, which was similar between the 150 mg and 600 mg Q4W dose regimens.

In the phase 2b study present here, the key efficacy endpoint was the CDASI-A score. The estimated change from baseline in CDASI-A scores at Week 12 showed that, PF-06823859 at 150 mg or 600 mg differentiated from placebo with similar treatment effects between the doses (p<0.0001 for both doses). Specifically, patients receiving 600 mg Q4W had a placebo-adjusted difference from baseline in CDASI-A score of −16.3 while 150 mg Q4W had a placebo-adjusted difference in CDASI-A score of −13.7. Additionally, PF-06823859 600 mg had numerically better efficacy scores than placebo across all key muscle function endpoints (TIS, MMT8, PtGA of myositis and CK) with nominal statistical significance for PtGA (p=0.0046) and CK (p=0.0282). Both doses of PF-06823859 (150 mg and 600 mg) administered every 4 weeks in Study C0251002 were generally well tolerated and safe in all stages of the study. Additionally, no safety signals were identified, and no dose relationship was observed in the TEAEs all causalities in all stages in the study presented here.

### DM Patients

Modeling of PK, PD, and efficacy in DM participants in the phase 2b study presented here supports selection of the 600 mg Q4W regimen in the Phase 3 study. Data from the Phase 1 Study and the Phase 2 Study enabled development of a population PK/PD model to characterize the relationship between drug exposure, target engagement (total IFNβ, GS), and IP-10, a PD biomarker (see Examples). Given the understanding of near complete target engagement provided by the PK/PD modeling for 600 mg Q4W, high specificity of PF-06823859 for IFNβ, an acceptable safety profile and the absence of observed off-target safety concerns from previous studies (mainly from C0251002), the 600 mg Q4W regimen provides high confidence for efficacy on both skin and muscle endpoints.

In addition, an exposure-response model was developed to characterize the relationship between PF-06823859 exposure and clinical efficacy endpoints for skin and muscle. The modeling results support the following key conclusions for DM participants:
IFNß is highly bound (>99%) to PF-06823859 at the exposure corresponding to the lower dose of 150 mg Q4W (evaluated in skin predominant DM in Stage 2), indicating near complete target engagement at 150 mg Q4W and higher doses. This is consistent with the observed clinical efficacy response for skin predominant disease, in terms of reduction in CDASI-A score, which was similar between the 150 mg and 600 mg Q4W dose regimens.
Inhibition of IP-10 was high at both 150 mg and 600 mg Q4W doses, and a small increase in % inhibition of IP-10 was predicted between the 150 mg and 600 mg doses. IP-10 is a biomarker downstream of IFNβ inhibition that is correlated with inflammation.
While near complete target engagement was shown at the lower dose 150 mg Q4W in the skin-predominant cohort in terms of CDASI-A clinical efficacy, the same dose was not studied in the muscle-predominant cohort. The muscle efficacy endpoints cannot be assumed based on skin efficacy endpoints due to limited understanding of correlation between 2 cohorts.

### PM Patients

PM and DM patients share the cardinal feature of muscle weakness. Further, the Type 1 IFN inducible genes are elevated in blood and muscle from patients with PM. Given the understanding of the high level of target engagement and demonstrated clinical efficacy for 600 mg Q4W dose in DM patients, the same dose is expected to achieve a similar level of efficacy in PM patients. Nevertheless, the sample size for the PM Cohort is slightly larger than that for DM/JDM thereby allowing for a slightly lower efficacy response relative to that in DM/JDM

### SLE and Other Indications

Based on the analyses provided herein, PF-06823859 demonstrates surprisingly good potential for application in a number of additional indications in particular, SLE, Cutaneous Lupus, and Psoriasis, and shows precision medicine potential for Ulcerative Colitis, Crohn's Disease, Rheumatoid Arthritis, Atopic Dermatitis and Scleroderma.

In some aspects, the disclosure provides a method for treating IIM in a patient, the method comprising administering to the patient an anti-IFNß antibody in a dosing regimen sufficient to improve signs and symptoms of IIM by at least 4 weeks after the start of treatment with the anti-IFNß antibody, said dosing regimen comprising a plurality of individual doses separated from each other by at least 2 weeks.

In some aspects, the IIM is one or more conditions selected from the group consisting of dermatomyositis, polymyositis, inclusion body myositis, and juvenile dermatomyositis. In some aspects, the IIM is dermatomyositis. In some aspects, the IIM is polymyositis. In some aspects, the IIM is inclusion body myositis. In some aspects, the IIM is juvenile dermatomyositis.

In some aspects, the disclosure provides a method for treating a patient with one or more conditions selected from the group consisting of SLE, Cutaneous Lupus, Psoriasis, Ulcerative Colitis, Crohn's Disease, Rheumatoid Arthritis, Atopic Dermatitis and Scleroderma, the method comprising administering to the patient an anti-IFNß antibody in a dosing regimen sufficient to improve signs and symptoms of the one or more condition by at least 4 weeks after the start of treatment with the anti-IFNß antibody, said dosing regimen comprising a plurality of individual doses separated from each other by at least 2 weeks.

In some aspects, the disclosure provides a method for treating a patient with one or more conditions selected from the group consisting of SLE, Cutaneous Lupus, and Psoriasis, the method comprising administering to the patient an anti-IFNß antibody in a dosing regimen sufficient to improve signs and symptoms of the one or more condition by at least 4 weeks after the start of treatment with the anti-IFNß antibody, said dosing regimen comprising a plurality of individual doses separated from each other by at least 2 weeks.

In some aspects, the disclosure provides a precision method for treating a patient with one or more conditions selected from the group consisting of Ulcerative Colitis, Crohn's Disease, Rheumatoid Arthritis, Atopic Dermatitis and Scleroderma, the method comprising administering to the patient an anti-IFNß antibody in a dosing regimen sufficient to improve signs and symptoms of the one or more condition by at least 4 weeks after the start of treatment with the anti-IFNß antibody, said dosing regimen comprising a plurality of individual doses separated from each other by at least 2 weeks.

In some aspects, the disclosure provides a method for treating SLE in a patient, the method comprising administering to the patient an anti-IFNß antibody in a dosing regimen sufficient to improve signs and symptoms of SLE by at least 4 weeks after the start of treatment with the anti-IFNß antibody, said dosing regimen comprising a plurality of individual doses separated from each other by at least 2 weeks.

In some aspects, the disclosure provides a method for treating Cutaneous Lupus in a patient, the method comprising administering to the patient an anti-IFN& antibody in a dosing regimen sufficient to improve signs and symptoms of Cutaneous Lupus by at least 4 weeks after the start of treatment with the anti-IFNß antibody, said dosing regimen comprising a plurality of individual doses separated from each other by at least 2 weeks.

In some aspects, the disclosure provides a method for treating Psoriasis in a patient, the method comprising administering to the patient an anti-IFN& antibody in a dosing regimen sufficient to improve signs and symptoms of Psoriasis by at least 4 weeks after the start of treatment with the anti-IFNß antibody, said dosing regimen comprising a plurality of individual doses separated from each other by at least 2 weeks.

In some aspects, the individual doses are separated from each other by at least a time period selected from the group consisting of 4 weeks, 1 month, 8 weeks, 2 months, 12 weeks, and 3 months, Favorably, the individual doses are separated from each other by the same time interval.

In some aspects, one or more of the individual doses are at an amount within a range whose lower limit is selected from the group consisting of about 25 mg, 50 mg, 100 mg, 150 mg, 200 mg, 250 mg, 300 mg, 350 mg, 400 mg, 450 mg, 500 mg, 550 mg, and 600 mg, and whose upper limit is selected from the group consisting of 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, and 1000 mg. The doses may be at least or at an amount selected from the group consisting of 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, and 1000 mg. In some aspects, the dose is 150 mg. In some aspects, the dose is 300 mg. In some aspects, the dose is about 600 mg. In some aspects, the dose is about 900 mg. Favorably, the individual doses are at the same amount.

In some aspects, the dosing regimen is continued for a duration of or at least a duration of selected from the group consisting of about 4 weeks, 1 month, 8 weeks, 2 months, 12 weeks, 3 months, 16 weeks, 4 months, 20 weeks, 5 months, 24 weeks, 6 months, and 26 weeks.

In some aspects of the disclosure, the improvement in signs or symptoms is assessed at a time point from the start of the dosing regimen selected from the group consisting of about 4 weeks, 1 month, 8 weeks, 2 months, 12 weeks, 3 months, 16 weeks, 4 months, 20 weeks, 5 months, 24 weeks, 6 months, and 26 weeks.

In some aspects of the disclosure, the improvement in signs or symptoms is maintained for a maintenance period of time, starting at a time point from the start of the dosing regimen selected from the group consisting of about 4 weeks, 1 month, 8 weeks, 2 months, 12 weeks, 3 months, 16 weeks, 4 months, 20 weeks, 5 months, 24 weeks, 6 months, and 26 weeks. In some aspects of the disclosure, the maintenance period of time is selected from the group consisting of 4 weeks, 1 month, 8 weeks, 2 months, 12 weeks, 3 months, 16 weeks, 4 months, 20 weeks, 5 months, 24 weeks, 6 months, and 26 weeks.

In some aspects of the disclosure, at least 4 weeks following the beginning of the dosing regimen the patient experiences an improvement in signs and symptoms that are maintained for at least a further period of time selected from the group consisting of about 4 weeks, 6 weeks, 8 weeks, 12 weeks, 16 weeks, 20 weeks, 24 weeks, and 26 weeks.

In some aspects of the disclosure, at least 8 weeks following the beginning of the dosing regimen the patient experiences an improvement in signs and symptoms that are maintained for at least a further period of time selected from the group consisting of about 4 weeks, 6 weeks, 8 weeks, 12 weeks, 16 weeks, 20 weeks, 24 weeks, and 26 weeks.

In some aspects of the disclosure, at least 12 weeks following the beginning of the dosing regimen the patient experiences an improvement in signs and symptoms that are maintained for at least a further period of time selected from the group consisting of about 4 weeks, 6 weeks, 8 weeks, 12 weeks, 16 weeks, 20 weeks, 24 weeks, and 26 weeks.

In some aspects of the disclosure, at least 16 weeks following the beginning of the dosing regimen the patient experiences an improvement in signs and symptoms that are maintained for at least a further period of time selected from the group consisting of about 4 weeks, 6 weeks, 8 weeks, 12 weeks, 16 weeks, 20 weeks, 24 weeks, and 26 weeks.

In some aspects of the disclosure, at least 24 weeks following the beginning of the dosing regimen the patient experiences an improvement in signs and symptoms that are maintained for at least a further period of time selected from the group consisting of about 4 weeks, 6 weeks, 8 weeks, 12 weeks, 16 weeks, 20 weeks, 24 weeks, and 26 weeks.

In some aspects of the disclosure, the improvement in signs or symptoms is characterized by a clinical response.

The clinical response may be characterized by one or more means selected from the group consisting of:
(i) A change from baseline of Manual Muscle Testing (MMT-8) score of greater than zero;
(ii) Total Improvement Score (TIS) of greater than zero;
(iii) A change from baseline in Patient Global Assessment score of less than zero;
(iv) An improvement in absolute muscle enzyme creatinine kinase of less than zero; and
(v) A change from baseline in Cutaneous Dermatomyositis Disease Area and Severity Index (CDASI-A) less than zero.

In some aspects of the disclosure, the change from baseline in Manual Muscle Testing (MMT-8) is characterized by a MMT-8 score of at least a value selected from the group consisting of 5, 6, 7, 8, 9, 10, 11, 12, 13, 14, 15, 16, 17, 18, 19, and 20.

In some aspects of the disclosure, the improvement in Total Improvement Score (TIS) is characterized by at least a TIS value selected from the group consisting of 20, 25, 30, 35, 40, 45, 50, 55, 60, 65, and 70. Clinical response defined as minimal improvement for DM comprises a TIS of at least 20. Clinical response defined as minimal improvement for JDM comprises a TIS of at least 30. Clinical response defined as moderate improvement for DM comprises a TIS of at least 40. Clinical response defined as moderate improvement for JDM comprises a TIS of at least 45. Clinical response defined as major improvement for DM comprises a TIS of at least 60. Clinical response defined as major improvement for JDM comprises a TIS of at least 70. The TIS may be placebo corrected. Where the clinical response is characterized by a placebo corrected TIS, the TIS is greater than zero.

In some aspects of the disclosure, the change from baseline in Patient Global Assessment is characterized by PtGA score of no greater than a value selected from the group consisting of −1, −2, −3, −4, and −5.

In some aspects of the disclosure, the change from baseline in absolute muscle enzyme creatinine kinase is characterized by an amount of no greater than a value selected from the group consisting of −75, −80, −85, −90, −95, −100, −105, −110, −115, −120, −125, −130, −135, −140, −145, −150, −155, −160, −165, −170, −175, −180, and −185.

In some aspects of the disclosure, the change from baseline in Cutaneous Dermatomyositis Disease Area and Severity Index (CDASI-A) is characterized by a CDASI-A score no greater a value than selected from the group consisting of −6, −7, −8, −9, −10, −11, −12, −13, −14, −15, 16, 17, 18, 19, and −20.

In some aspects of the disclosure, the patient experiences an improvement in signs or symptoms after starting the dosing regimen within a period of time selected from the group consisting of about 4 weeks, 6 weeks, 8 weeks, 10 weeks, 12 weeks, 16 weeks, 20 weeks, 24 weeks, and 26 weeks.

In some aspects of the disclosure, the patient shows a clinical response after starting the dosing regimen within a period of time selected from the group consisting of about 4 weeks, 6 weeks, 8 weeks, 10 weeks, 12 weeks, 16 weeks, 20 weeks, 24 weeks, and 26 weeks.

In some aspects of the disclosure, the anti-IFNß antibody comprises three CDRs from the variable heavy chain region having the sequence shown in SEQ ID NO: 3 and three CDRs from the variable light chain region having the sequence shown in SEQ ID NO: 4.

In some aspects of the disclosure, the anti-IFNß antibody comprises a HCDR1 having the sequence shown in SEQ ID NO: 5, a HCDR2 having the sequence shown in SEQ ID NO: 6, a HCDR3 having the sequence shown in SEQ ID NO: 7, a LCDR1 having the sequence shown in SEQ ID NO: 8, a LCDR2 having the sequence shown in SEQ ID NO: 9, and a LCDR3 having the sequence shown in SEQ ID NO:10.

In some aspects of the disclosure, the anti-IFNß antibody comprises a variable heavy chain region having the sequence shown in SEQ ID NO: 3 and a variable light chain region having the sequence shown in SEQ ID NO: 4.

In some aspects of the disclosure, the anti-IFNß antibody comprises a heavy chain having the sequence shown in SEQ ID NO: 1 and a light chain having the sequence shown in SEQ ID NO: 2. In some aspects of the disclosure, the C-terminal lysine (K) of the heavy chain amino acid sequence of SEQ ID NO: 1 is optional.

In some aspects of the disclosure, the anti-IFNß antibody comprises a VH encoded by the nucleic acid sequence of the insert of the vector deposited as CTI-AF1-VH having ATCC accession number PTA-122727 and a VL encoded by the nucleic acid sequence of the insert of the vector deposited as CTI-AF1-VL having ATCC accession number PTA-122726. In some aspects of the disclosure, the anti-IFN& antibody comprises the VH sequence encoded by the insert in the plasmid deposited with the ATCC and having ATCC Accession No. PTA-122727. In some aspects of the disclosure, the anti-IFNß antibody comprises the VL sequence encoded by the insert in the plasmid deposited with the ATCC and having ATCC Accession No. PTA-122726.

In some aspects of the disclosure, the anti-IFNß antibody competes for binding with an anti-IFNß antibody comprising a variable heavy chain region having the sequence shown in SEQ ID NO: 3 and a variable light chain region having the sequence shown in SEQ ID NO: 4.

In some aspects of the disclosure, the anti-IFNß antibody competes for binding with an antibody comprising a VH encoded by the nucleic acid sequence of the insert of the vector deposited as CTI-AF1-VH having ATCC accession number PTA-122727 and a VL encoded by the nucleic acid sequence of the insert of the vector deposited as CTI-AF1-VL having ATCC accession number PTA-122726.

In some aspects of the disclosure, an anti-IFNß antibody may be used for the preparation of a medicament for a method of treatment according any of those set provided herein.

### General Techniques

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### DEFINITIONS

The following terms, unless otherwise indicated, shall be understood to have the following meanings:

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)₂, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy-chain constant regions that correspond to the different classes of immunoglobulins are called alpha, delta, epsilon, gamma, and mu, respectively. The subunit structures and three-dimensional configurations of different classes of immunoglobulins are well known.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

As known in the art, a “constant region” of an antibody refers to the constant region of the antibody light chain or the constant region of the antibody heavy chain, either alone or in combination.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a target (e.g., INFβ) epitope is an antibody that binds this epitope with greater affinity, avidity, more readily, and/or with greater duration than it binds to other target epitopes or non-target epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

As used in the art, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976, J. Immunol., 117:587; and Kim et al., 1994, J. Immunol., 24:249).

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

As used herein, “treatment” is an approach for obtaining beneficial or desired clinical results. For purposes of this invention, beneficial or desired clinical results include reduction or improvement in signs and symptoms of osteoarthritis, for example as compared to before administration of the anti-IFNß antibody.

“Ameliorating” means a lessening or improvement of one and more signs or symptoms of osteoarthritis, for example as compared to not administering an anti-IFNß antibody as described herein. “Ameliorating” also includes shortening or reduction in duration of a symptom.

As used herein, an “effective dosage” or “effective amount” of drug, compound, or pharmaceutical composition is an amount sufficient to effect any one or more beneficial or desired results. In more specific aspects, an effective amount prevents, alleviates or ameliorates signs or symptoms of myositis, and/or prolongs the survival of the subject being treated. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing one or more signs or symptoms of myositis, decreasing the dose of other medications required to treat the disease, enhancing the effect of another medication, and/or delaying the progression of the disease in patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

Treatment “effectively improves” or “effectively reduces” when assessment of the sign or symptom of IIM is quantified via a clinical measure relative to baseline and during and/or after the treatment period. The difference between the clinical measure at baseline and during/after treatment is compared and used to determine whether the sign or symptom has improved and the treatment is effective. This comparison can include comparison to placebo or to one or more of the prior therapies.

A “patient”, an “individual” or a “subject”, used interchangeably herein, is a mammal, more preferably, a human. Mammals also include, but are not limited to, farm animals (e.g., cows, pigs, horses, chickens, etc.), sport animals, pets, primates, horses, dogs, cats, mice and rats.

As used herein, “pharmaceutically acceptable carrier” or “pharmaceutical acceptable excipient” includes any material which, when combined with an active ingredient, allows the ingredient to retain biological activity and is non-reactive with the subject's immune system.

Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, PA, 1990; and Remington, The Science and Practice of Pharmacy 20th Ed. Mack Publishing, 2000).

Reference to “about” a value or parameter herein includes (and describes) embodiments that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g. within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

The term “subcutaneous administration” refers to the administration of a substance into the subcutaneous layer.

The term “preventing” or “prevent” refers to (a) keeping a disorder from occurring or (b) delaying the onset of a disorder or onset of symptoms of a disorder.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control.

Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present invention. The materials, methods, and examples are illustrative only and not intended to be limiting.

### Anti-IFNß Antibodies

TABLE 3
Sequences of exemplary antibodies of the invention.

| SEQ ID NO: | SEQUENCE |
|---|---|
| SEQ ID NO: 1 | QVQLVQSGAE VKKPGSSVKV SCKASGYTFS RYWMHWVRQA PGQGLEWMGH |
| PF-06823859 HC | IDPSDSYTYY NQKFKGRVTI TADESTSTAY MELSSLRSED TAVYYCARWD |
| | YGNLLFEYWG QGTLVTVSSa stkgpsvfpl apsskstsgg taalgclvkd |
| | yfpepvtvsw nsgaltsgvh tfpavlqssg lyslssvvtv pssslgtqty |
| | icnvnhkpsn tkvdkkvepk scdkthtcpp cpapeaagap svflfppkpk |
| | dtlmisrtpe vtcvvvdvsh edpevkfnwy vdgvevhnak tkpreeqyns |
| | tyrvvsvltv lhqdwlngke ykckvsnkal papiektisk akgqprepqv |
| | ytlppsreem tknqvsltcl vkgfypsdia vewesngqpe nnykttppvl |
| | dsdgsfflys kltvdksrwq qgnvfscsvm healhnhytq kslslspg(k) |
| SEQ ID NO: 2 | DIQMTQSPSS LSASVGDRVT ITCRTSQDIG NYLNWYQQKP GKAFKLLIYS |
| PF-06823859 LC | TSRLHSGVPS RFSGSGSGTD FTLTISSLQP EDFATYYCQQ GIILPITFGG |
| | GTKVEIKrtv aapsvfifpp sdeqlksgta svvcllnnfy preakvqwkv |
| | dnalqsgnsq esvteqdskd styslsstlt lskadyekhk vyacevthqg |
| | lsspvtksfn rgec |
| SEQ ID NO: 3 | QVQLVQSGAE VKKPGSSVKV SCKASGYTFS RYWMHWVRQA PGQGLEWMGH |
| PF-06823859 VH | IDPSDSYTYY NQKFKGRVTI TADESTSTAY MELSSLRSED TAVYYCARWD |
| | YGNLLFEYWG QGTLVTVSS |
| SEQ ID NO: 4 | DIQMTQSPSS LSASVGDRVT ITCRTSQDIG NYLNWYQQKP GKAFKLLIYS |
| PF-06823859 VL | TSRLHSGVPS RFSGSGSGTD FTLTISSLQP EDFATYYCQQ GIILPITFGG |
| | GTKVEIK |
| SEQ ID NO: 5 | GYTFS RYWMH |
| PF-06823859 HCDR1 | |
| SEQ ID NO: 6 | HIDPSDSYTYYNQKEKG |
| PF-06823859 HCDR2 | |
| SEQ ID NO: 7 | WD YGNLLFEY |
| PF-06823859 HCDR3 | |
| SEQ ID NO: 8 | RTSQDIGNYLN |
| PF-06823859 LCDR1 | |
| SEQ ID NO: 9 | STSRLHS |
| PF-06823859 LCDR2 | |
| SEQ ID NO: 10 | QQGIILPIT |
| PF-06823859 LCDR3 | |
| SEQ ID NO: 11 | a stkgpsvfpl apsskstsgg taalgclvkd yfpepvtvsw nsgaltsgvh |
| PF-06823859 HC | tfpavlqssg lyslssvvtv pssslgtqty icnvnhkpsn tkvdkkvepk |
| constant domain | scdkthtcpp cpapeaagap svflfppkpk dtlmisrtpe vtcvvvdvsh |
| | edpevkfnwy vdgvevhnak tkpreeqyns tyrvvsvltv lhqdwlngke |
| | ykckvsnkal papiektisk akgqprepqv ytlppsreem tknqvsltcl |
| | vkgfypsdia vewesngqpe nnykttppvl dsdgsfflys kltvdksrwq |
| | qgnvfscsvm healhnhytq kslslspg(k) |
| SEQ ID NO: 12 | rtv aapsvfifpp sdeqlksgta svvcllnnfy preakvqwkv |
| PF-06823859 LC | dnalqsgnsq esvteqdskd styslsstlt lskadyekhk vyacevthqg |
| constant domain | lsspvtksfn rgec |
| SEQ ID NO: 13 | CAGGTGCAGCTGGTGCAGAGCGGCGCCGAGGTGAAGAAGCCCGGCAGCAGCGTGAA |
| PF-06823859 VH | GGTGAGCTGCAAGGCCAGCGGCTACACCTTCAGCCGGTACTGGATGCACTGGGTGC |
| nucleic acid | GGCAGGCCCCCGGCCAGGGCCTGGAGTGGATGGGCCACATCGACCCCAGCGACAGC |
| | TACACCTACTACAACCAGAAGTTCAAGGGCCGGGTGACCATCACCGCCGACGAGAG |
| | CACCAGCACCGCCTACATGGAGCTGAGCAGCCTGCGGAGCGAGGACACCGCCGTGT |
| | ACTACTGCGCCCGGTGGGACTACGGCAACCTGCTGTTCGAGTACTGGGGCCAGGGC |
| | ACCCTGGTGACCGTCTCGAGC |
| SEQ ID NO: 14 | GACATCCAGATGACCCAGAGCCCCAGCAGCCTGAGCGCCAGCGTGGGCGACCGGGT |
| PF-06823859 VL | GACCATCACCTGCCGGACCAGCCAGGACATCGGCAACTACCTGAACTGGTACCAGC |
| nucleic acid | AGAAGCCCGGCAAGGCCTTCAAGCTGCTGATCTACAGCACCAGCCGGCTGCACAGC |
| | GGCGTGCCCAGCCGGTTCAGCGGCAGCGGCAGCGGCACCGACTTCACCCTGACCAT |
| | CAGCAGCCTGCAGCCCGAGGACTTCGCCACCTACTACTGCCAGCAGGGGATTATTT |
| | TGCCCATTACCTTCGGCGGCGGCACCAAGGTGGAGATCAAG |

The antibodies as described herein can be made by any method known in the art. For the production of hybridoma cell lines, the route and schedule of immunization of the host animal are generally in keeping with established and conventional techniques for antibody stimulation and production, as further described herein. General techniques for production of human and mouse antibodies are known in the art and/or are described herein.

It is contemplated that any mammalian subject including humans or antibody producing cells therefrom can be manipulated to serve as the basis for production of mammalian, including human and hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C., Nature 256:495-497, 1975 or as modified by Buck, D. W., et al., In Vitro, 18:377-381, 1982. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies.

Hybridomas that produce antibodies used for the present invention may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity, if present, can be removed, for example, by running the preparation over adsorbents made of the immunogen attached to a solid phase and eluting or releasing the desired antibodies off the immunogen. Immunization of a host animal with cells expressing the antibody target (e.g., IFNß), a human target protein (e.g., IFNß), or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups, can yield a population of antibodies (e.g., monoclonal antibodies).

If desired, the antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., J. Immunol. Methods 329, 112, 2008; U.S. Pat. No. 7,314,622.

In some embodiments, antibodies may be made using hybridoma technology. It is contemplated that any mammalian subject including humans or antibody producing cells therefrom can be manipulated to serve as the basis for production of mammalian, including human, hybridoma cell lines. The route and schedule of immunization of the host animal are generally in keeping with established and conventional techniques for antibody stimulation and production, as further described herein. Typically, the host animal is inoculated intraperitoneally, intramuscularly, orally, subcutaneously, intraplantar, and/or intradermally with an amount of immunogen, including as described herein.

In some embodiments, antibodies as described herein are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation pattern of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, variations in the glycosylation pattern of the antibodies can be expected (see, e.g. Hse et al., 1997, J. Biol. Chem. 272:9062-9070).

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example, using endoglycosidase H (Endo H), N-glycosidase F, endoglycosidase F1, endoglycosidase F2, endoglycosidase F3. In addition, the recombinant host cell can be genetically engineered to be defective in processing certain types of polysaccharides. These and similar techniques are well known in the art.

Other methods of modification include using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art, some of which are described below and in the Examples.

### Polynucleotides, Vectors, and Host Cells

The invention also provides polynucleotides encoding any of the anti-IFNß antibodies as described herein. Polynucleotides can be made and expressed by procedures known in the art.

In another aspect, the invention provides compositions (such as a pharmaceutical compositions) comprising any of the polynucleotides of the invention, for use in one or more methods of the invention. In some embodiments, the composition comprises an expression vector comprising a polynucleotide encoding any of the anti-IFNß antibodies described herein, for use in one or more methods of the invention.

In another aspect, provided is an isolated cell line that produces the anti-IFNß antibodies as described herein for use in one or more methods of the invention.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes an antibody or a fragment thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants preferably exhibit at least about 70% identity, more preferably, at least about 80% identity, yet more preferably, at least about 90% identity, and most preferably, at least about 95% identity to a polynucleotide sequence that encodes a native antibody or a fragment thereof.

### Compositions

The invention also provides pharmaceutical compositions comprising an effective amount of an anti-IFNß antibody as described herein, and such pharmaceutical compositions for use in methods of treatment as described herein. Examples of such compositions, as well as how to formulate, are also described herein. It is understood that the compositions can comprise more than one anti-IFNß antibody.

The composition used in the present invention can further comprise pharmaceutically acceptable carriers, excipients, or stabilizers (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

The anti-IFNß antibody, and compositions thereof, can also be used in conjunction with, or administered separately, simultaneously, or sequentially with other agents that serve to enhance and/or complement the effectiveness of the agents.

### Formulations

The antibody, or antigen-binding fragment thereof, of the invention can be formulated as a pharmaceutical composition. The pharmaceutical composition may further comprise a pharmaceutically acceptable carrier, excipient, and/or stabilizer (Remington: The Science and practice of Pharmacy 20th Ed., 2000, Lippincott Williams and Wilkins, Ed. K. E. Hoover), in the form of lyophilized formulation or aqueous solution. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG). Pharmaceutically acceptable excipients are further described herein.

Therapeutic formulations of the anti-IFNß3 antibody used in accordance with the present invention are prepared for storage by mixing the protein having the desired degree of purity with optional pharmaceutically acceptable carriers, excipients or stabilizers (Remington, The Science and Practice of Pharmacy 20th Ed. Mack Publishing, 2000), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™ PLURONICS™ or polyethylene glycol (PEG).

The formulations to be used for in vivo administration must be sterile. This is readily accomplished by, for example, filtration through sterile filtration membranes. Therapeutic anti-IFNß antibody compositions are generally placed into a container having a sterile access port, for example, an intravenous solution bag or vial having a stopper pierceable by a hypodermic injection needle.

In one aspect, provided is a stable aqueous formulation comprising: at least 25 mg/ml to about 200 mg/ml of an anti-IGFß antibody, or antigen-binding fragment thereof; a buffer; a polyol; a surfactant; a stabilizer, optionally a chelating agent; and wherein the formulation has a pH at about 5.0 to about 6.5. The formulation described herein have an extended shelf life, preferably of at least or more than about 36 months (e.g. at about 5 °C). Formulations described herein are particularly useful for use in the methods and uses as described herein.

In some aspects, the present disclosure provides an aqueous formulation comprising: an anti-IFNß antibody at a concentration of between about 25 mg·mL and about 200 mg·mL; Histidine or His-HCL at a concentration of between 10 and 50 mM; Arginine or NaCL in an amount 20-150 mM, a polyol, (which may favorably be Sucrose or Trehalose in an amount between 20 mg/ml and 85 mg/ml); At a pH of between pH 5.0 and pH 6.5. In some aspects, the antibody optionally further comprises a chelator.

In some embodiments, the antibody can be selected from the group consisting of monoclonal antibodies, polyclonal antibodies, antibody fragments (e.g., Fab, Fab′, F(ab′)2, Fv, Fc, ScFv etc.), chimeric antibodies, bispecific antibodies, heteroconjugate antibodies, single chain (ScFv), mutants thereof, fusion proteins comprising an antibody portion (e.g., a domain antibody), humanized antibodies, human antibodies, and any other modified configuration of the immunoglobulin molecule that comprises an antigen recognition site of the required specificity, including glycosylation variants of antibodies, amino acid sequence variants of antibodies, and covalently modified antibodies. The antibody may be murine, rat, human, or any other origin (including chimeric or humanized antibodies). In some embodiments, the antibody can be human but is more preferably humanized. Preferably the antibody is isolated, further preferably it is substantially pure. Where the antibody is an antibody fragment this preferably retains the functional characteristics of the original antibody i.e. the ligand binding and/or antagonist or agonist activity. In some embodiments, the antibody heavy chain constant region may be from any type of constant region, such as IgG, IgM, Igo, IgA, and IgE; and any isotypes, such as IgG1, IgG2, IgG3, and IgG4. Preferably the antibody is an IgG1 antibody.

In some aspects, the IGFß antibody comprises three CDRs from the variable heavy chain region having the sequence shown in SEQ ID NO: 3 and three CDRs from the variable light chain region having the sequence shown in SEQ ID NO: 4. In some aspects, the anti-IFNß antibody comprises a HCDR1 having the sequence shown in SEQ ID NO: 5, a HCDR2 having the sequence shown in SEQ ID NO: 6, a HCDR3 having the sequence shown in SEQ ID NO: 7, a LCDR1 having the sequence shown in SEQ ID NO: 8, a LCDR2 having the sequence shown in SEQ ID NO: 9, and a LCDR3 having the sequence shown in SEQ ID NO:10. In some aspects, the anti-IFNß antibody comprises a variable heavy chain region having the sequence shown in SEQ ID NO: 3 and a variable light chain region having the sequence shown in SEQ ID NO: 4. In some aspects, the anti-IFNß antibody comprises a heavy chain having the sequence shown in SEQ ID NO: 1 and a light chain having the sequence shown in SEQ ID NO: 2, wherein the C-terminal lysine (K) of the heavy chain amino acid sequence of SEQ ID NO: 1 is optional. In some aspects, the anti-IFNß antibody comprises a VH encoded by the nucleic acid sequence of the insert of the vector deposited as CTI-AF1-VH having ATCC accession number PTA-122727 and a VL encoded by the nucleic acid sequence of the insert of the vector deposited as CTI-AF1-VL having ATCC accession number PTA-122726. In some aspects, the antibody comprises the VH sequence encoded by the insert in the plasmid deposited with the ATCC and having ATCC Accession No. PTA-122727. In some aspects, the antibody comprises the VL sequence encoded by the insert in the plasmid deposited with the ATCC and having ATCC Accession No. PTA-122726. In some aspects, the anti-IFNß antibody competes for binding with an anti-IFNß antibody comprising a variable heavy chain region having the sequence shown in SEQ ID NO: 3 and a variable light chain region having the sequence shown in SEQ ID NO: 4. In some aspects, the anti-IFNß antibody competes for binding with an antibody comprising a VH encoded by the nucleic acid sequence of the insert of the vector deposited as CTI-AF1-VH having ATCC accession number PTA-122727 and a VL encoded by the nucleic acid sequence of the insert of the vector deposited as CTI-AF1-VL having ATCC accession number PTA-122726.

The antibody may be present in the formulation at a concentration ranging from about 25 mg/ml to about 200 mg/ml, from about 40 mg/ml to 200 mg/ml, from about 50 mg/ml to about 175 mg/ml, or from about 60 mg/ml to about 150 mg/ml.

The antibody may be present in the formulation at a concentration of about 50 mg·ml. The antibody may be present in the formulation at a concentration of about 60 mg·ml. In some aspects, the antibody is present in an amount of between 50 and 70 mg·mL. In some aspects, the antibody is present at an amount of about 60 mg·mL. Such concentrations are particularly suitable for IV dosing. In some aspects, the antibody dosing regimen comprises 600 mg of antibody every 4 weeks by IV injection. The 600 mg of such a dosing regimen may favorably be provided at a concentration of 60 mg·mL to reduce viscosity and minimize drug product wastage and dose pooling. The formulation of the present disclosure is engineered to provide a stable and safe dose for both SC and IV formulation.

The antibody may be present in the formulation at a concentration about 80 mg·ml. The antibody may be present in the formulation at a concentration of about 100 mg·ml. The antibody may be present in the formulation at a concentration of about 120 mg·mL. The antibody may be present in the formulation at a concentration of between about 120 mg·mL and about 175 mg·mL. The antibody may be present in the formulation at a concentration of about 140 mg·ml. The antibody may be present in the formulation at a concentration of between about 141 and about 154 mg·mL. The antibody may be present in the formulation at a concentration of about 150 mg·ml.

In some aspects, the antibody is present in an amount of between 140 and 160 mg·mL. The antibody may be present in the formulation at a concentration of about 140 mg·ml. In some aspects, the antibody is present in an amount of between 141 and 154 mg·mL. In some aspects, the antibody is present at an amount of about 150 mg·mL. Such concentrations are particularly suitable for SC dosing. In some aspects, the antibody dosing regimen comprises 600 mg of antibody every week by SC injection. The 600 mg of such a dosing regimen may favorably be provided at a concentration of 150 mg·mL to minimize local injection volume. The formulation of the present disclosure is engineered to provide a stable and safe dose for both SC and IV formulation.

In some aspects, the buffer (His or His-HCL) provides the formulation with a pH close to physiological pH for reduced risk of pain or anaphylactoid side effects on injection and also provides enhanced antibody stability and resistance to aggregation, oxidation, and fragmentation. In some aspects, the buffer is His. In some aspects, the buffer is His-HCL.

The concentration of the buffer can range from about 1 millimolar (mM) to about 100 mM. Preferably, the concentration of the buffer is from about 5 mM to about 50 mM, further preferably about 10 mM to about 30 mM, more preferably about 15 mM to about 25 mM. Preferably, the concentration of the buffer is about 1 mM, about 2 mM, 20 about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 30 mM, about 35 mM, about 40 mM, about 45 mM, about 50 mM, about 55 mM, about 60 mM, about 65 25 mM, about 70 mM, about 75 mM, about 80 mM, about 85 mM, about 90 mM, about 95 mM, or about 100 mM. In some embodiment, the buffer is a His buffer in the concentration of about 20 mM.

The concentration of the stabilizer can range from about 1 millimolar (mM) to about 100 mM. Preferably, the concentration of the stabilizer is from about 10 mM to about 90 mM, further preferably about 25 mM to about 75 mM, more preferably about 40 mM to about 60 mM. Preferably, the concentration of the stabilizer is about 1 mM, about 2 mM, 20 about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 30 mM, about 35 mM, about 40 mM, about 45 mM, about 50 mM, about 55 mM, about 60 mM, about 65 25 mM, about 70 mM, about 75 mM, about 80 mM, about 85 mM, about 90 mM, about 95 mM, or about 100 mM. In some embodiment, the stabilizer is present at a concentration of about 50 mM.

In some aspects, the stabilizer is Arginine or NaCL. In some aspects, the stabilizer is Arginine. In some aspects, the stabilizer is Arginine at a concentration of about 1 mM to about 100 mM. In some aspects, the stabilizer is Arginine at a concentration of about 25 mM to about 75 mM. In some aspects, the stabilizer is Arginine at a concentration of about 50 mM to about 60 mM. In some aspects, the stabilizer is Arginine at a concentration of about 50 mM.

In some embodiments, the polyol can have a molecular weight that, for example without limitation, is less than about 600 kD (e.g., in the range from about 120 to about 400 kD), and comprises multiple hydroxyl groups including sugars (e.g., reducing and nonreducing sugars or mixtures thereof, saccharide, or a carbohydrate), sugar alcohols, sugar acids, or a salt or mixtures thereof. Examples of non-reducing sugar, include, but 20 are not limited to, sucrose, trehalose, and mixtures thereof. In some embodiments, the polyol is mannitol, trehalose, sorbitol, erythritol, isomalt, lactitol, maltitol, xylitol, glycerol, lactitol, propylene glycol, polyethylene glycol, inositol, or mixtures thereof. In other embodiments, the polyol can be, for example without limitation, a monosaccharide, disaccharide or polysaccharide, or mixtures of any of the foregoing. The saccharide or 25 carbohydrate can be, for example without limitation, fructose, glucose, mannose, sucrose, sorbose, xylose, lactose, maltose, sucrose, dextran, pullulan, dextrin, cyclodextrins, soluble starch, hydroxyethyl starch, water-soluble glucans, or mixtures thereof. The polyol may be sucrose or trehalose. The polyol may be trehalose. The polyol may be sucrose.

In some aspects, the concentration of the polyol in the formulation ranges from about 1 mg/ml to about 300 mg/ml, from about 1 mg/ml to about 200 mg/ml, or from about 1 mg/ml to about 120 mg/ml. Preferably the concentration of the polyol in the formulation is about 10 mg/ml to about 100 mg/ml, from about 20 mg/ml to about 70 mg/ml, or from about 40 mg/ml to about 60 mg/ml). In some aspects, the concentration of the polyol in the formulation is about 1 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, about 51 mg/ml, about 52 mg/ml, about 53 mg/ml, about 54 mg/ml, about 55 mg/ml, about 56 mg/ml, about 57 mg/ml, about 58 mg/ml, about 59 mg/ml, about 60 mg/ml, about 65 mg/ml, about 70 mg/ml, about 75 mg/ml, about 80 mg/ml, about 81 mg/ml, about 82 mg/ml, about 83 mg/ml, about 84 mg/ml, about 85 mg/ml, about 86 mg/ml, about 87 mg/ml, about 88 mg/ml, about 89 mg/ml, about 90 mg/ml, about 91 mg/ml, about 92 mg/ml, about 93 mg/ml, about 94 mg/ml, about 95 mg/ml, about 96 mg/ml, about 97 mg/ml, about 98 mg/ml, about 99 mg/ml, about 100 mg/ml, about 101 mg/ml, about 102 mg/ml, about 103 mg/ml, about 104 mg/ml, about 105 mg/ml, about 106 mg/ml, about 107 mg/ml, about 108 mg/ml, about 109 mg/ml, about 110 mg/ml, about 111 mg/ml, about 112 mg/ml, about 113 mg/ml, about 114 mg/ml, about 115 mg/ml, about 116 mg/ml, about 117 mg/ml, about 118 mg/ml, about 119 mg/ml, about 120 mg/ml, about 121 mg/ml, about 122 mg/ml, about 123 mg/ml, about 124 mg/ml, about 125 mg/ml, about 126 mg/ml, about 127 mg/ml, about 128 mg/ml, about 129 mg/ml, about 130 mg/ml, about 131 mg/ml, about 132 mg/ml, about 133 mg/ml, about 134 mg/ml, about 135 mg/ml, about 136 mg/ml, about 137 mg/ml, about 138 mg/ml, about 139 mg/ml, about 140 mg/ml, about 141 mg/ml, about 142 mg/ml, about 143 mg/ml, about 144 mg/ml, about 145 mg/ml, about 146 mg/ml, about 147 mg/ml, about 148 mg/ml, about 149 mg/ml, or about 150 mg/ml. In some embodiments, the polyol is sucrose at a concentration of from about 1 mg/ml to about 300 mg/ml, from about 1 mg/ml to about 200 mg/ml, or from about 1 mg/ml to about 100 mg/ml. In some embodiments, the polyol is sucrose at a concentration of from about 10 mg/ml to about 90 mg/ml, from about 20 mg/ml to about 80 mg/ml, or from about 25 mg/ml to about 75 mg/ml. In some embodiments, the polyol is sucrose at a concentration of from about 40 mg/ml to about 60 mg/ml. Preferably the concentration of the sucrose is about 50 mg·mL.

Surfactants, as used in the present invention, can alter the surface tension of a liquid antibody formulation. In certain embodiments, the surfactant reduces the surface tension of a liquid antibody formulation. In still other embodiments, the surfactant can contribute to an improvement in stability of any of the antibody in the formulation. The surfactant can also reduce aggregation of the formulated antibody (e.g., during shipping and storage) and/or minimize the formation of particulates in the formulation and/or reduces adsorption (e.g., adsorption to a container). For example, the surfactant can also improve stability of the antibody during and after a freeze/thaw cycle. The surfactant can be, for example without limitation, a polysorbate, poloxamer, triton, sodium dodecyl sulfate, sodium laurel sulfate, sodium octyl glycoside, lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine, myristamidopropyl-dimethylamine, palmidopropyl-dimethylamine, isostearamidopropyl-dimethylamine, sodium methyl cocoyl-taurate, disodium methyl oleyl-taurate, dihydroxypropyl PEG 5 linoleammonium chloride, polyethylene glycol, polypropylene glycol, and mixtures thereof. The surfactant can be, for example without limitation, polysorbate 20, polysorbate 21, polysorbate 40, polysorbate 60, polysorbate 61, polysorbate 65, polysorbate 80, polysorbate 81, polysorbate 85, PEG3350 and mixtures thereof. In some aspects, the surfactant is polysorbate 80 (PS80).

The concentration of the surfactant generally ranges from about 0.01 mg/ml to about 10 mg/ml, from about 0.01 mg/ml to about 5.0 mg/ml, from about 0.01 mg/ml to about 2.0 mg/ml, from about 0.01 mg/ml to about 1.5 mg/ml, from about 0.01 mg/ml to about 1.0 mg/ml, from about 0.01 mg/ml to about 0.5 mg/ml, from about 0.01 mg/ml to about 0.4 mg/ml, from about 0.01 mg/ml to about 0.3 mg/ml, from about 0.01 mg/ml to 30 about 0.2 mg/ml, from about 0.01 mg/ml to about 0.15 mg/ml, from about 0.01 mg/ml to about 0.1 mg/ml, from about 0.01 mg/ml to about 0.05 mg/ml, from about 0.1 mg/ml to about 1 mg/ml, from about 0.1 mg/ml to about 0.5 mg/ml, or from about 0.1 mg/ml to about 0.3 mg/ml. Further preferably the concentration of the surfactant is about 0.05 mg/ml, about 0.06 mg/ml, about 0.07 mg/ml, about 0.08 mg/ml, about 0.09 mg/ml, about 0.1 mg/ml, about 0.15 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, or about 1 mg/ml. In some embodiments, the polysorbate is polysorbate 80 at a concentration ranging from about 0.1 mg/ml to about 0.3 mg/ml. In some aspects, the surfactant is PS80 and is present at an amount of 0.2 mg/ml.

The formulation may further comprise a chelator. Chelating agents can lower the formation of reduced oxygen species, reduce acidic species (e.g. deamidation) formation, reduce antibody aggregation, and/or reduce antibody fragmentation, and/or reduce antibody 10 oxidation in the formulation of the present invention. For example, the chelating agent can be a multidentate ligand that forms at least one bond (e.g., covalent, ionic, or otherwise) to a metal ion and acts as a stabilizer to complex with species, which might otherwise promote instability.

In some embodiments, the chelating agent can be selected from the group 15 consisting of aminopolycarboxylic acids, hydroxyaminocarboxylic acids, N-substituted glycines, 2-(2-amino-2-oxocthyl) aminoethane sulfonic acid (BES), deferoxamine (DEF), citric acid, niacinamide, and desoxycholates and mixtures thereof. In some embodiments, the chelating agent is selected from the group consisting of ethylenediaminetetraacetic acid (EDTA), diethylenetriamine pentaacetic acid 5 (DTPA), 20 nitrilotriacetic acid (NTA), N-2-acetamido-2-iminodiacetic acid (ADA), bis(aminoethyl)glycolether, N, N, N′, N′-tetraacetic acid (EGTA), transdiaminocyclohexane tetraacetic acid (DCTA), glutamic acid, and aspartic acid, Nhydroxyethyliminodiacetic acid (HIMDA), N, N-bis-hydroxyethylglycine (bicine) and N-(trishydroxymethylmethyl) 10 glycine (tricine), glycylglycine, sodium desoxycholate, 25 ethylenedia mine, propylenedia mine, diethylenetria mine, triethylenetetraa mine (trien), disodium edetate dihydrate (or disodium EDTA dihydrate or EDTA disodium salt), calcium EDTA oxalic acid, malate, citric acid, citric acid monohydrate, and trisodium citrate-dihydrate, 8-hydroxyquinolate, amino acids, histidine, cysteine, methionine, peptides, polypeptides, and proteins and mixtures thereof.

In some embodiments, the chelating agent is selected from the group consisting of salts of EDTA including dipotassium edetate, disodium edetate, edetate calcium disodium, sodium edetate, trisodium edetate, and potassium edetate; and a suitable salt of deferoxamine (DEF) is deferoxamine mesylate (DFM), or mixtures thereof. Chelating agents as used herein may be the free acid or free base form or salt form of the compound, also as an anhydrous, solvated or hydrated form of the compound or corresponding salt. The chelator may be EDTA.

The chelator may be present at an amount of between 0.01 and 0.1 mg/ml. The chelator may be present at an amount of between 0.02 and 0.08 mg/ml.

The chelator may be present at an amount of 0.05 mg/ml. The chelator may be present and may be EDTA present in an amount of 0.05 mg·mL.

According to some embodiments, the pH can be in the range of about pH 5.0 to about 6.6, preferably between about pH 5.0 to 6.5 or about 5.0 to 6.0, and most preferably between pH 5.2 to 5.8. The pH for the formulation of the present disclosure can be in the range selected from between any one of about pH 5.2, 5.3, 5.4, 5.5, or 5.6 and any one of about pH 6.5, 6.4, 6.3, 6.2, 6.1, 6.0, 5.9, 5.8 or 5.7. In some embodiments the pH can be selected from pH values of any of about pH 5.0, 5.1, 5.2, 5.3, 5.4, 5.5, 5.6, 5.7, 5.8, 5.9, 6.0, 6.1, 6.2, 6.3, 6.4, and 6.5, Preferably, the pH is pH 5.8+/−0.5, and most preferably, the pH is pH 5.8+/−0.3.

In some embodiments the formulation can comprise a preservative. Preferably the preservative agent is selected from phenol, m-cresol, benzyl alcohol, benzalkonium chloride, benzalthonium chloride, phenoxyethanol and methyl paraben. The concentration of the preservative generally ranges from about 0.001 mg/ml to about 50 mg/ml, from about 0.005 mg/ml to about 15.0 mg/ml, from about 0.008 10 mg/ml to about 12.0 mg/ml or from about 0.01 mg/ml to about 10.0 mg/ml. Preferably the concentration of preservative can be about 0.1 mg/ml, 0.2 mg/ml, 0.3 mg/ml, about 0.4 mg/ml, about 0.5 mg/ml, about 0.6 mg/ml, about 0.7 mg/ml, 0.8 mg/ml, 0.9 mg/ml about 1.0 mg/ml, 2.0 mg/ml, 3.0 mg/ml, about 4.0 mg/ml, about 5.0 mg/ml, about 6.0 mg/ml, about 7.0 mg/ml, 8.0 mg/ml, 9.0 mg/ml about 9.1 mg/ml, about 9.2 mg/ml, 9.3 15 mg/ml, 9.4 mg/ml, 9.5 mg/ml, 9.6 mg/ml, 9.7 mg/ml, 9.8 mg/ml, 9.9 mg/ml, 10.0 mg/ml. Most preferably, the concentration of preservative is about 0.1 mg/ml or 9.0 mg/mL. In some embodiments, the formulation does not contain a preservative.

### Kits

The invention also provides kits comprising any or all of the anti-IFNß antibodies described herein. Kits of the invention include one or more containers comprising an anti-IFNß antibody described herein and instructions for use in accordance with any of the methods of the invention described herein. Generally, these instructions comprise a description of administration of the anti-IFNß antibody for the above described therapeutic treatments. In some embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

The instructions relating to the use of an anti-IFNß antibody generally include information as to dosage, dosing schedule, and route of administration for the intended treatment. The containers may be unit doses, bulk packages (e.g., multi-dose packages) or subunit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-IFNß antibody. The container may further comprise a second pharmaceutically active agent.

Kits may optionally provide additional components such as buffers and interpretive information. Normally, the kit comprises a container and a label or package insert(s) on or associated with the container.

### BIOLOGICAL DEPOSIT

Representative materials of the present invention were deposited in the American Type Culture Collection, 10801 University Boulevard, Manassas, Va. 20110-2209, USA, on Dec. 18, 2015. Vector CTI-AF1-VH, having ATCC Accession No. PTA-122727, comprises a DNA insert encoding the heavy chain variable region of antibody CTI-AF1, and vector CTI-AF1-VL, having ATCC Accession No. PTA-122726, comprises a DNA insert encoding the light chain variable region of antibody CTI-AF1. The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ATCC under the terms of the Budapest Treaty, and subject to an agreement between Pfizer Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638).

The owner of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

### EXAMPLES

### Example 1 Study Design to Evaluate Anti-INFβ Antibody

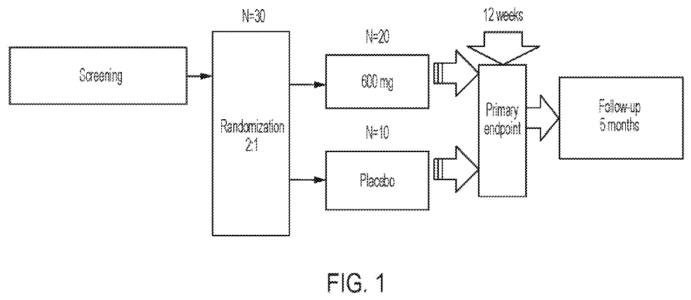

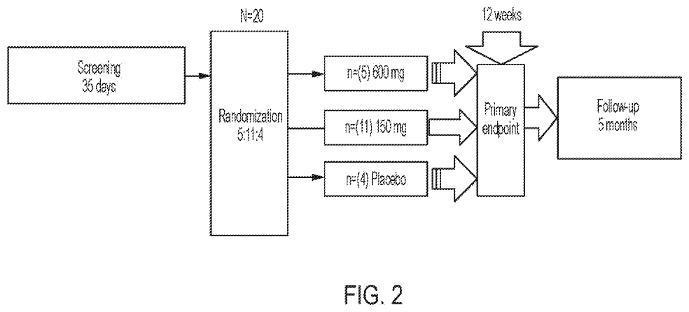

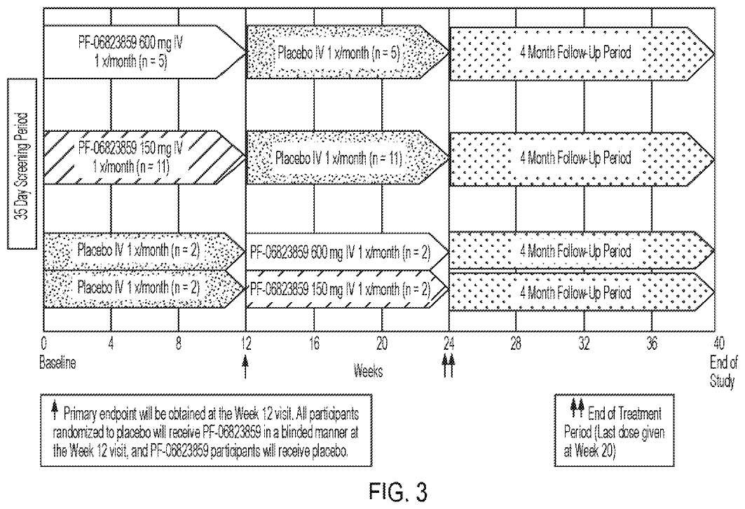

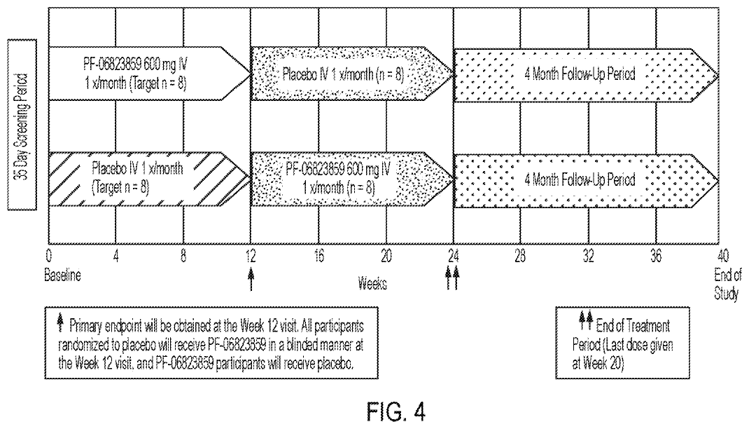

C0251002 is an ongoing, double-blind, placebo-controlled, multi-stage, multi-center Phase 2 study to evaluate the efficacy, safety, and tolerability of PF-06823859 in adult participants with moderate to severe DM. The study consists of 4 stages. Stages 1, Stage 2, and Amended Stage 2 included participants with skin disease predominant DM and Stage 3 included participants with muscle predominant DM. FIGS. 1,2, 3, 4 show the initial designs and planned sample sizes for each stage. The actual number of individuals randomized is provided in the text below.

Stage 1: Participants with skin involvement (CDASI-Activity 214 at screening) who failed at least 1 standard of care systemic treatment, (eg, corticosteroids) were randomized to receive 600 mg of PF-06823859 or placebo in a 2:1 ratio. Investigational drug or placebo administration took place on Day 1, Week 4, and Week 8. The primary endpoint (CFB CDASI-A) was assessed at Week 12.

Stage 2: Participants with skin involvement (CDASI-Activity ≥14 at screening) were randomized to receive 600 mg of PF-06823859, 150 mg of PF-06823859, or placebo in a 5:11:4 ratio. Investigational drug or placebo administration took place on Day 1, Week 4, and Week 8 of the study. The primary endpoint (CFB CDASI-A) was assessed at Week 12.

Amended Stage 2: A fixed sequence design was employed in Amended Stage 2 to provide all study participants with the opportunity to receive active drug during the treatment period. Participants were randomized to one of the following sequences in a 5:11:2:2 ratio: 600 mg PF-06823859 then placebo, 150 mg PF-06823859 then placebo, placebo then 600 mg PF-06823859, or placebo then 150 mg PF-06823859. Investigational drug or placebo administration (as dictated by the treatment sequence) occurred on Day 1, Week 4, Week 8, Week 12, Week 16, and Week 20. The primary endpoint (CFB CDASI-A) was assessed at Week 12 of Amended Stage 2. After the treatment period ended at Week 24, participants then entered a 4-month follow-up period or rolled over to the long-term extension study, C0251008.

Stage 3: A fixed sequence design was also employed in Stage 3 where participants with predominantly muscle involvement were randomized to one of the following sequences in a 1:1 ratio: 600 mg PF-06823859 then placebo, or placebo then 600 mg PF-06823859 with a treatment switch at Week 12. The inclusion criteria for the muscle involvement required that the subject met one of the following two criteria: (1) MMT-8 5136/150 and PhGA (VAS ≥3 cm on 0-10 cm scale) or (2) sum of PhGA, PtGA, and extramuscular global assessment is ≥10 cm (using 0-10 cm VAS scale for each) and had failed at least two or more adequate courses of an immunosuppressive or immunomodulatory agent, including Intravenous Immunoglobulin (IVIG). Immunosuppressive and immunomodulatory agents including IVIG in stable doses were allowed as concomitant medications. Investigational drug or placebo administration (as dictated by the treatment sequence) occurred on Day 1, Week 4, Week 8, Week 12, Week 16, and Week 20. The secondary endpoint (TIS) was assessed longitudinally at weeks 4, 8 and 12 (Week 12 being the key timepoint). Other secondary muscle-related endpoints are: MMT-8 and PtGA of Myositis. After Week 12 they were switched to the other treatment in the sequence. After the treatment period ended at Week 24, participants entered a 4-month follow-up period or rolled over to the long-term extension study, C0251008.

### Example 2 Selected Endpoints for Evaluating the Efficacy of Anti-INFβ Antibody

### Primary Endpoints

1. Stage 1: The primary objective was to evaluate the efficacy of PF-06823859 as measured by change from baseline (CFB) of CDASI-A score at Week 12. Formal statistical testing occurred in Stage 1 only.
2. Stage 2 and Amended Stage 2: The primary objective was to evaluate the efficacy of PF-06823859 as measured by change from baseline (CFB) of CDASI-A score at Week 12 in a pooled efficacy analysis of Stage 1, Stage 2, and Amended Stage 2 data from baseline to Week 12.
3. Stage 3: The primary objective was to evaluate the safety and tolerability of PF-06823859 as measured by incidence of AEs, laboratory abnormalities, changes in vital signs, and ECG findings.

### Secondary Endpoints

Skin Cohort (Pooled Data from Stage 1, Stage 2, and Amended Stage 2) [Week 0-Week 12]
4. CFB and percent CFB in CDASI-A scores over time
5. Proportion of CDASI-A responders (CFB in CDASI-A >5, Percent CFB in CDASI-A >40%) at Week 12
6. Safety and tolerability as measured by incidence of AEs, laboratory abnormalities, changes in vital signs, and ECG findings

### Muscle Cohort [Week 0-Week 12] (Stage 3)

7. TIS
8. Endpoints related to muscle disease
   1. Manual Muscle Testing (MMT-8)
   2. Patient Global Assessment of myositis (PtGA)
   3. Physician Global Assessment of myositis (PhGA)
   4. Muscle Damage biomarker: Creatine Kinase (CK) levels

### Statistical Analysis Populations and Methods

The primary analysis populations for safety and efficacy are as follows:
9. Full Analysis Set in Stage 1 (FAS1) includes all participants who received at least one dose of randomized treatment in Stage 1.
10. Full Analysis Set in Stage 3 (FAS3) includes all participants who received at least one dose of randomized treatment in Stage 3.
11. Pooled Full Analysis Set for skin disease predominant stages (PFASS) includes all subjects who received at least one dose of randomized treatment in Stage 1, Stage 2, or Amended Stage 2.
12. Safety Analysis Set (SAS) includes all subjects who received at least one dose of randomized treatment. Subsets of the SAS specific to Stage 1, Stage 2, Amended Stage 2, and Stage 3 are abbreviated SAS1, SAS2, SASA2, and SAS3 respectively.

In the Stage 3 muscle cohort, the TIS/CK at Week 12 was assessed using an MMRM model using data at all visits post-baseline up to Week 12. This model uses TIS/CK as an outcome and fixed effects for treatment, time (visit), treatment by time, and unstructured covariance matrix. CFB in CDASI-A/MMT-8/PtGA/PhGA/CK at Week 12 was assessed using a LANCOVA model using data at all visits post-baseline up to Week 12. This model uses CFB in CDASI as the outcome and baseline value, treatment, time (visit), and treatment by time as covariates. The unstructured covariance matrix is used. In both MMRM and LANCOVA analyses (except for the Stage 1 comparison between changes from baseline of CDASI-A at Week 12) the p-values are exploratory and were not adjusted for the multiplicity of comparisons.

### Example 3 Results

### Study Population, Disposition, and Demography

### Muscle Cohort

A total of 18 participants with muscle disease predominant DM were randomized (and treated) from 5 countries (U.S., Poland, Hungary, Spain, Germany). A total of 18 participants completed the 12-week treatment period, only 6 (33.3%) completed the 24-week treatment period. Table 4 shows the disposition summary of participants in Stage 3.

### Skin Cohort

A total of 32, 9, and 16 participants with skin disease predominant DM were randomized (and treated) respectively from 2 countries (United States, Hungary). Table 5 shows the number of participants randomized, completed, and discontinued from the 12-week treatment period and follow-up period in Stage 1 and Stage 2. Table 6 shows the number of participants randomized, completed, and discontinued from the 24-week treatment period (also broken up by pre- and post-12 weeks) and follow-up period in Amended Stage 2. The discontinuation rate across the first 12 weeks in Stage 1, Stage 2, Amended Stage 2, and Stage 3 were 9.4%, 0%, 0% and 0%.

TABLE 4
Participant Disposition and Evaluation
by Treatment Group in Stage 3.

| | Placebo then | | 600 mg | | | |
|---|---|---|---|---|---|---|
| | 600 mg | | PF-06823859 | | | |
| | PF-06823859 | | then Placebo | | Total | |
| | (N = 9) | | (N = 9) | | (N = 18) | |
| | n (%) | | n (%) | | n (%) | |
| Number (%) of | | | | | | |
| Participants | | | | | | |
| Assigned to treatment | 9 | (100.0) | 9 | (100.0) | 18 | (100.0) |
| Treated | 9 | (100.0) | 9 | (100.0) | 18 | (100.0) |
| Not treated | 0 | | 0 | | 0 | |
| Disposition phase: | | | | | | |
| Baseline to Week 12 | | | | | | |
| Participants entered: | 9 | (100.0) | 9 | (100.0) | 18 | (100.0) |
| Discontinued | 0 | | 0 | | 0 | |
| Completed | 4 | (44.4) | 3 | (33.3) | 7 | (38.9) |
| Ongoing/unknown | 5 | (55.6) | 6 | (66.7) | 11 | (61.1) |
| Disposition phase: | | | | | | |
| Week 12 to Week 24 | | | | | | |
| Participants entered: | 9 | (100.0) | 9 | (100.0) | 18 | (100.0) |
| Discontinued | 0 | | 1 | (11.1) | 1 | (5.6) |
| Completed | 4 | (44.4) | 2 | (22.2) | 6 | (33.3) |
| Ongoing/unknown | 5 | (55.6) | 6 | (66.7) | 11 | (61.1) |
| Disposition phase: | | | | | | |
| follow-up | | | | | | |
| Participants entered: | 1 | (11.1) | 1 | (11.1) | 2 | (11.1) |
| Discontinued | 0 | | 0 | | 0 | |
| Completed | 0 | | 0 | | 0 | |
| Ongoing/unknown | 1 | (11.1) | 1 | (11.1) | 2 | (11.1) |

TABLE 5
Participant Disposition and Evaluation by Treatment Group in Stage 1 and Stage 2.

| | Stage 1 | | | | | | | Stage 2 | | |
|---|---|---|---|---|---|---|---|---|---|---|
| | | | PF- | | | | | PF- | | |
| | | | 06823859 | | | | | 06823859 | PF- | |
| Number (%) | Placebo | | 600 mg | | Total | | Placebo | 150 mg | 06823859 | Total |
| of | (N = 10) | | (N = 22) | | (N = 32) | | (N = 1) | (N = 5) | 600 mg (N = 3) | (N = 9) |
| Participants | n (%) | | n (%) | | n (%) | | n (%) | n (%) | n (%) | n (%) |
| Assigned to | 10 | | 22 | | 32 | | 1 | 5 | 3 | 9 |
| treatment | | | | | | | | | | |
| | (100.0) | | (100.0) | | (100.0) | | (100.0) | (100.0) | (100.0) | (100.0) |
| Treated | 10 | | 22 | | 32 | | 1 | 5 | 3 | 9 |
| | (100.0) | | (100.0) | | (100.0) | | (100.0) | (100.0) | (100.0) | (100.0) |
| Not treated | 0 | | 0 | | 0 | | 0 | 0 | 0 | 0 |
| Disposition | | | | | | | | | | |
| phase: | | | | | | | | | | |
| Baseline to | | | | | | | | | | |
| Week 12 | | | | | | | | | | |
| Participants | 10 | | 22 | | 32 | | 1 | 5 | 3 | 9 |
| entered: | | | | | | | | | | |
| | (100.0) | | (100.0) | | (100.0) | | (100.0) | (100.0) | (100.0) | (100.0) |
| Discontinued | 1 | (10.0) | 2 | (9.1) | 3 | (9.4) | 0 | 0 | 0 | 0 |
| Reason for | | | | | | | | | | |
| discontinuation | | | | | | | | | | |
| Withdrawal by | 0 | | 1 | (4.5) | 1 | (3.1) | 0 | 0 | 0 | 0 |
| subject | | | | | | | | | | |
| Completed | 9 | | 20 | | 29 | | 1 | 5 | 3 | 9 |
| | (90.0) | | (90.9) | | (90.6) | | (100.0) | (100.0) | (100.0) | (100.0) |
| Ongoing | 0 | | 0 | | 0 | | 0 | 0 | 0 | 0 |
| Disposition | | | | | | | | | | |
| phase: | | | | | | | | | | |
| follow-up | | | | | | | | | | |
| Participants | 10 | | 22 | | 32 | | 1 | 5 | 3 | 9 |
| entered: | | | | | | | | | | |
| | (100.0) | | (100.0) | | (100.0) | | (100.0) | (100.0) | (100.0) | (100.0) |
| Discontinued | 2 | (20.0) | 1 | (4.5) | 3 | (9.4) | 0 | 0 | 0 | 0 |
| Reason for | | | | | | | | | | |
| discontinuation | | | | | | | | | | |
| Adverse event | 1 | | 0 | | 1 | | 0 | 0 | 0 | 0 |
| | (10.0) | | | | (3.1) | | | | | |
| Withdrawal by | 1 | | 1 | | 2 | | 0 | 0 | 0 | 0 |
| subject | | | | | | | | | | |
| | (10.0) | | (4.5) | | (6.3) | | | | | |
| Completed | 8 | (80.0) | 21 | (95.5) | 29 | (90.6) | 1 (100.0) | 5 (100.0) | 3 (100.0) | 9 (100.0) |

TABLE 6
Participant Disposition and Evaluation by Treatment Group in Amended Stage 2.

| | | | | | Amended | | | | | |
|---|---|---|---|---|---|---|---|---|---|---|
| | | | | | Stage 2 | | | | | |
| | Placebo then | | Placebo then | | 150 mg | | 600 mg | | | |
| | 150 mg | | 600 mg | | PF-06823859 | | PF-06823859 | | | |
| | PF-06823859 | | PF-06823859 | | then Placebo | | then Placebo | | Total | |
| | (N = 2) | | (N = 1) | | (N = 10) | | (N = 3) | | (N = 16) | |
| | n (%) | | n (%) | | n (%) | | n (%) | | n (%) | |
| Number (%) of | | | | | | | | | | |
| Participants | | | | | | | | | | |
| Assigned to | 2 | (100.0) | 1 | (100.0) | 10 | (100.0) | 3 | (100.0) | 16 | (100.0) |
| Treatment | | | | | | | | | | |
| Treated | 2 | (100.0) | 1 | (100.0) | 10 | (100.0) | 3 | (100.0) | 16 | (100.0) |
| Disposition phase: | | | | | | | | | | |
| Baseline to Week 12 | | | | | | | | | | |
| Participants entered: | 2 | (100.0) | 1 | (100.0) | 10 | (100.0) | 3 | (100.0) | 16 | (100.0) |
| Discontinued | 0 | | 0 | | 0 | | 0 | | 0 | |
| Completed | 2 | (100.0) | 1 | (100.0) | 9 | (90.0) | 3 | (100.0) | 15 | (93.8) |
| Ongoing | 0 | | 0 | | 1 | (10.0) | 0 | | 1 | (6.3) |
| Disposition phase: | | | | | | | | | | |
| Week 12 to Week 24 | | | | | | | | | | |
| Participants entered: | 2 | (100.0) | 1 | (100.0) | 10 | (100.0) | 3 | (100.0) | 16 | (100.0) |
| Discontinued | 0 | | 0 | | 1 | (10.0) | 1 | (33.3) | 2 | (12.5) |
| Reason for | | | | | | | | | | |
| discontinuation | | | | | | | | | | |
| Other than lost | 0 | | 0 | | 1 | (10.0) | 1 | (33.3) | 2 | (12.5) |
| to follow-up | | | | | | | | | | |
| or withdrawal | | | | | | | | | | |
| by subject | | | | | | | | | | |
| Completed | 2 | (100.0) | 1 | (100.0) | 8 | (80.0) | 2 | (66.7) | 13 | (81.3) |
| Ongoing | 0 | | 0 | | 1 | (10.0) | 0 | | 1 | (6.3) |
| Disposition phase: | | | | | | | | | | |
| follow-up | | | | | | | | | | |
| Participants entered: | 1 | (50.0) | 0 | | 3 | (30.0) | 3 | (100.0) | 7 | (43.8) |
| Discontinued | 0 | | 0 | | 0 | | 2 | (66.7) | 2 | (12.5) |
| Reason for | | | | | | | | | | |
| discontinuation | | | | | | | | | | |
| Lost to follow-up | 0 | | 0 | | 0 | | 1 | (33.3) | 1 | (6.3) |
| Withdrawal by | 0 | | 0 | | 0 | | 1 | (33.3) | 1 | (6.3) |
| subject | | | | | | | | | | |

### Demography and Baseline Disease Characteristics

### Muscle Cohort

Baseline characteristics for each treatment sequence in Stage 3 were generally balanced and are summarized in Table 7. The majority of the patients were white (89%), and female (72%) and the median disease duration was 2 years. The concomitant (ongoing) medications included oral steroids (66.7%), IVIG (38.9%) and immunosuppressive drugs (e.g., MMF, AZA, MTX) (77.8%), as a single treatment or in-combination.

### Skin Cohort

Baseline characteristics for each treatment sequence in Stage 1, Stage 2, and Amended Stage 2 were generally balanced and are summarized in Tables 8 and 9. The majority of the patients were white (90.6%, 100%, 93.8% in Stage 1, Stage 2, and Amended Stage 2, respectively) and female (90.6%, 100%, 93.8% respectively). In the overall population, 53%, 55.6%, and 50% of participants had ongoing steroid use in Stage 1, Stage 2, and Amended Stage 2, respectively while 12.5%, 44.4%, and 12.5% had ongoing IVIG use.

TABLE 7
Demographic and Baseline Characteristics in Stage 3.

| | Stage 3 | | | | | |
|---|---|---|---|---|---|---|
| | | | PF-06823859 | | | |
| | Placebo | | 600 mg | | Total | |
| Characteristics | (N = 9) | | (N = 9) | | (N = 18) | |
| Age (years), | 47.4 | (12.1) | 42.4 | (16.7) | 44.9 | (14.4) |
| mean (sd) | | | | | | |
| Females (%) | 66.7 | | 77.8 | | 72.2 | |
| Whites (%) | 88.9 | | 88.9 | | 88.9 | |
| Weight (kg), | 77.4 | (15.3) | 72.7 | (17.8) | 75.0 | (16.3) |
| mean (sd) | | | | | | |
| Disease duration | 6.5 | (8.8) | 3.9 | (4.4) | 5.2 | (6.9) |
| (years), mean (sd) | | | | | | |
| Prior steroid use (%) | 77.8 | | 77.8 | | 77.8 | |
| Prior IVIG use (%) | 33.3 | | 55.6 | | 44.4 | |
| Baseline steroid | 66.7 | | 55.6 | | 61.1 | |
| use (%) | | | | | | |
| Median prednisone | 7.5 | | 7.5 | | 7.5 | |
| equivalent dose of | | | | | | |
| steroids at baseline | | | | | | |
| (mg) | | | | | | |
| Baseline IVIG | 22.2 | | 44.4 | | 33.3 | |
| use (%) | | | | | | |
| CDASI-A, mean (sd) | 17.2 | (11.6) | 12.6 | (8.1) | 14.9 | (10.0) |
| CDASI-A >=14 (%) | 44.4 | | 55.6 | | 50.0 | |
| MMT-8, mean (sd) | 117.0 | (19.6) | 103.9 | (22.0) | 110.4 | (21.5) |
| PtGA (mm), | 55.6 | (18.0) | 69.1 | (16.7) | 62.4 | (18.2) |
| mean (sd) | | | | | | |
| CK (U/L), mean (sd) | 227.9 | (270.1) | 321.2 | (391.9) | 274.6 | (476.0) |

TABLE 8
Demographic and Baseline Characteristics in Stage 1 and Stage 2.

| | Stage 1 | | | | | | Stage 2 | | | | | | | |
|---|---|---|---|---|---|---|---|---|---|---|---|---|---|---|
| | | | PF- | | | | | | PF- | | | | | |
| | | | 06823859 | | | | | | 06823859 | | PF- | | | |
| | Placebo | | 600 mg | | Total | | Placebo | | 150 mg | | 06823859 | | | |
| | (N = 10) | | (N = 22) | | (N = 32) | | (N = 1) | | (N = 5) | | 600 mg (N = 3) | | Total (N = 9) | |
| Age | 50.2 | (14.0) | 54.4 | (13.2) | 53.1 | (13.4) | 42.0 | (—) | 51.6 | (15.7) | 45.7 | (23.7) | 48.6 | (16.7) |
| (years), | | | | | | | | | | | | | | |
| mean (sd) | | | | | | | | | | | | | | |
| Females | 90.0 | | 90.9 | | 90.6 | | 100 | | 100 | | 100.0 | | 100.0 | |
| (%) | | | | | | | | | | | | | | |
| Whites (%) | 90.0 | | 90.9 | | 90.6 | | 100 | | 100 | | 100.0 | | 100.0 | |
| Weight | 76.0 | (18.5) | 80.8 | (22.4) | 79.3 | (21.1) | 85.0 | (—) | 77.0 | (16.9) | 88.3 | (10.3) | 81.7 | (14.2) |
| (kg) mean | | | | | | | | | | | | | | |
| Disease | 7.5 | (7.6) | 6.5 | (5.0) | 6.8 | (5.8) | 0.8 | (—) | 6.3 | (7.0) | 6.6 | (9.4) | 5.8 | (7.1) |
| duration | | | | | | | | | | | | | | |
| (years), | | | | | | | | | | | | | | |
| mean (sd) | | | | | | | | | | | | | | |
| Prior | 50.0 | | 72.7 | | 65.6 | | 100.0 | | 80.0 | | 0.0 | | 55.6 | |
| steroid use | | | | | | | | | | | | | | |
| (%) | | | | | | | | | | | | | | |
| Prior IVIG | 40.0 | | 13.6 | | 21.9 | | 0.0 | | 20.0 | | 66.7 | | 33.3 | |
| use (%) | | | | | | | | | | | | | | |
| Baseline | 30 | | 54.5 | | 46.9 | | 100.0 | | 80.0 | | 0 | | 55.6 | |
| steroid use | | | | | | | | | | | | | | |
| (%) | | | | | | | | | | | | | | |
| Median | 5.0 | | 10.0 | | 5.0 | | 2.5 | | 5.0 | | (—) | | 3.8 | |
| prednisone | | | | | | | | | | | | | | |
| equivalent | | | | | | | | | | | | | | |
| dose of | | | | | | | | | | | | | | |
| steroid at | | | | | | | | | | | | | | |
| baseline | | | | | | | | | | | | | | |
| (mg) | | | | | | | | | | | | | | |
| Baseline | 20.0 | | 9.1 | | 12.5 | | 0.0 | | 20.0 | | 66.7 | | 33.3 | |
| IVIG use | | | | | | | | | | | | | | |
| (%) | | | | | | | | | | | | | | |
| CDASI-A, | 31.5 | (11.8) | 33.2 | (10.3) | 32.7 | (10.6) | 23.0 | (—) | 28.6 | (9.0) | 37.0 | (9.6) | 30.8 | (9.4) |
| mean (sd) | | | | | | | | | | | | | | |

TABLE 9
Demographic and Baseline Characteristics in Amended Stage 2.

| | Amended Stage 2 | | | | |
|---|---|---|---|---|---|
| | | | | 600 mg PF- | |
| | | | 150 mg PF- | 06823859 | |
| | Placebo then 150 | Placebo then 600 | 06823859 | then | |
| | mg PF- 06823859 | mg PF- 06823859 | then Placebo | Placebo | Total |
| | (N = 2) | (N = 1) | (N = 10) | (N = 3) | (N = 16) |
| Age (years), mean (sd) | 64.0 (1.4) | 44.0 (—) | 53.9 (11.0) | 47.0 (13.1) | 53.2 |
| Females (%) | 100 | 100 | 100 | 66.7 | 93.8 |
| Whites (%) | 100 | 100 | 90 | 100 | 93.8 |
| Weight (kg), mean (sd) | 72.2 (8.5) | 54.4 (—) | 80.4(23.6) | 75.7 (13.4) | 76.9 |
| Disease duration (years), | 8.4 (2.0) | 2.0 (—) | 6.0 (7.0) | 3.3 (3.7) | 5.5 (5.9) |
| mean(sd) | | | | | |
| Prior steroid use (%) | 50.0 | 100.0 | 60.0 | 66.7 | 62.5 |
| Prior IVIG use (%) | 0 | 0 | 30.0 | 0 | 18.8 |
| Baseline steroid use (%) | 50.0 | 100 | 40.0 | 66.7 | 50.0 |
| Median prednisone | 5.0 | 10.0 | 5.0 | 2.5 | 5.0 |
| equivalent dose of steroids | 5.0 | 10.0 | | | |
| at baseline (mg) | | | | | |
| Baseline IVIG use (%) | 100 | 0 | 20.0 | 0 | 12.5 |
| CDASI-A, mean (SD) | 26 (5.7) | 37.0 (—) | 35.4 (13.2) | 30.0 (7.9) | 33.3 |

Efficacy: Key Results & Supportive Findings Muscle Cohort

### Total Improvement Score (TIS)

The estimated mean (with 90% CI) of TIS scores at weeks 4, 8, 12 and estimated difference in mean scores between the active treatment and placebo arms are presented in the Table 10. The sample size of the trial was not large enough for the testing of the presence of the treatment effect (positive difference in the mean TIS values between active and placebo treatment groups) at Week 12 visit. At Week 12, the values of the mean TIS score for PF-06823859 and placebo were 56.4 (41.4, 71.4) and 36.9 (22.0, 52.0). The higher placebo effect may be due to ongoing concomitant medications, which included oral steroids (66.7%), IVIG (38.9%) and immunosuppressive drugs (e.g., MMF, AZA, MTX) (77.8%), as a single treatment or in-combination.

TABLE 10
Statistical Analysis for Absolute value of TIS (MMRM, Week 4-Week 12)

| | | | | Difference from placebo | |
|---|---|---|---|---|---|
| | | | 90% | | 90% |
| Analysis | Treatment during first | Least Squares | Confidence | Least Squares | Confidence |
| Visit | 12 weeks | Mean | Interval | Mean Difference | Interval |
| Week 4 | Placebo (N = 9) | 25.8 | [13.2, 38.5] | | |
| | PF-06823859 6-- | | | | |
| | PF-06823859 | 36.7 | [24.0, 49.4] | 10.8 | [−7.1, 28.8] |
| | 600 mg (N = 9) | | | | |
| Week 8 | Placebo (N = 9) | 36.7 | [23.4, 50.0] | | |
| | PF-06823859 600 mg | | | | |
| | PF-06823859 | 49.2 | [35.9, 62.5] | 12.5 | [−6.3, 31.3] |
| | 600 mg (N = 9) | | | | |
| Week 12 | Placebo (N = 9) | 36.9 | [22.0, 52.0] | | |
| | PF-06823859 600 mg | 56.4 | [41.4, 71.4] | 19.4 | [−1.8, 40.7] |
| | (N = 9) | | | | |

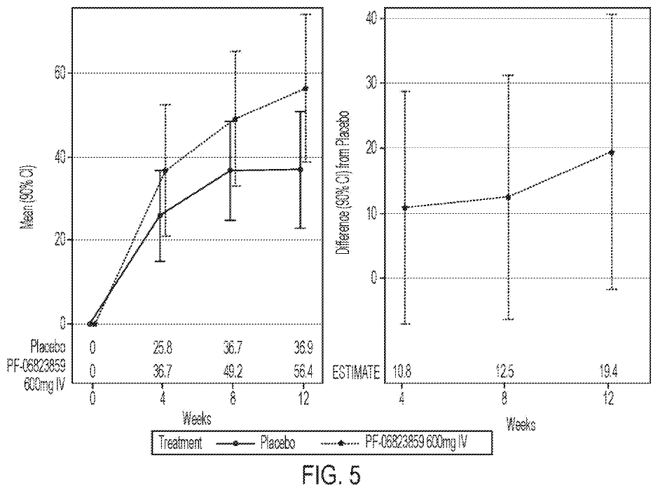

FIG. 5 shows the estimated mean TIS in each treatment group (left panel) and the difference between the mean TIS scores of the active and placebo groups (right panel) from baseline to Week 12. Note that the TIS score evaluates change from baseline and is not defined at the baseline (equals zero for each subject) so the values at Week 0 are shown for a reference only. The mean total improvement score (TIS) showed numerical advantage of PF-06823859 600 mg compared to placebo with increasing trends over time (0-12 week) and without plateauing at week 12.

The sensitivity analysis was conducted after removal of a single efficacy observation for a subject who took prohibited medications. The estimated mean TIS values at Week 12 were 56.4 (90% CI=(41.8, 71.0)) and 35.6 (90% CI=(20.9, 50.3)) for PF-06823859 and placebo, respectively, with a placebo-adjusted difference of 20.8 (90% CI=(0.0, 41.5)) and reached nominal statistical significance in TIS (p=0.0497). Sensitivity analyses also reached nominal significance.

These values and the value of the estimated treatment effect of 19.4 (−1.8, 40.7) are comparable to the estimate of the treatment effect in the Octagam (IVIG) study (26.8 (19.0, 34.6)).

### Other Secondary Endpoints for Muscle Function

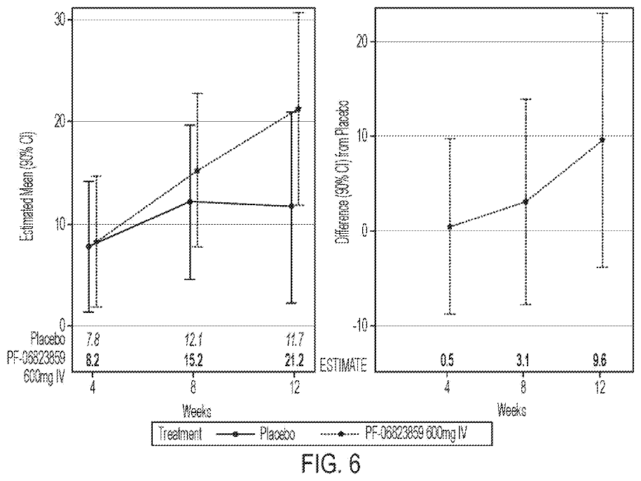

FIG. 6 shows the estimated mean of changes from baseline (90% CI) in MMT-8 (higher scores denote improvement). PF-06823859 600 mg shows an increasing trend over time. The achieved mean CFB values at Week 12 were 21.2 (11.9, 30.6) and 11.7 (2.3, 21.0) in the PF-06823859 600 mg and placebo treatment arms. The estimated treatment effect (90% CI) was 9.6 (−3.8, 23.0). These values are comparable to the estimate of the treatment effect for MMT-8 (11.2 (6.9, 15.5)) observed in the ProDERM study.

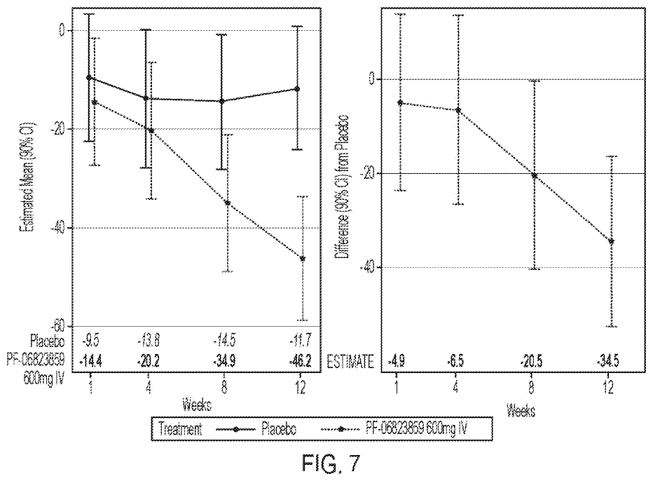

FIG. 7 shows the estimated mean of changes from baseline (with 90% CI) in Patient Global Assessment of Myositis. Lower scores denote improvement, and the range used in the plot is 0 to 100 (100 mm VAS, equivalent to 10 cm VAS). PF-06823859 600 mg shows a decreasing trend overtime (0-12 weeks) without reaching a plateau at week 12. The mean CFB values for PF-06823859 600 mg and placebo arms at Week 12 are −46.2 (−58.6, −33.7) and −11.7 (−24.1, 0.77). The estimated difference of the means is −34.5 (−52.7, −16.2). These values are numerically larger than the estimates (−11.0 (−18.7, −3.3)) of the treatment effect for PtGA observed in ProDERM study.

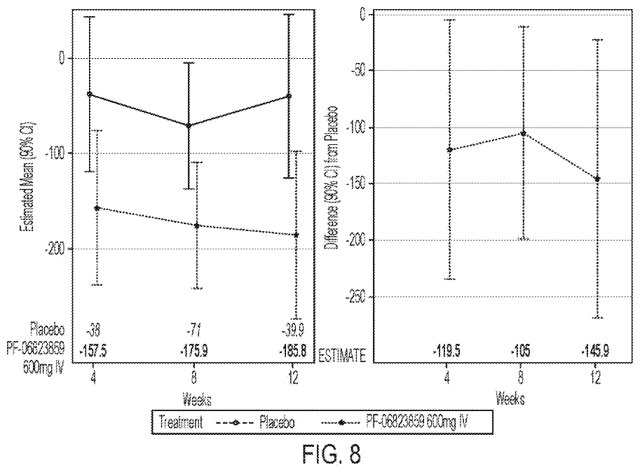

FIG. 8 shows the estimated mean change from baseline (90% CI) in Creatine kinase. The mean baseline CK for PF-06823859 600 mg and placebo were 321.2 and 227.9 respectively. On the CFB scale PF-06823859 600 mg shows a clear separation from placebo across Weeks 4 to weeks 12 and achieved −185.8 (−273.9, −97.6) at week 12 while placebo was −39.9 (−125.7, 46.0) with delta of −145.9 (−269.4, −22.4). This improvement in muscle enzyme in patients treated with PF-06823859 is consistent with the clinical improvement in muscle function, MMT-8 score and PtGA of myositis. The ProDERM study did not show a treatment effect on CK (estimate was 183.6 (−364.0, 731.2)).

Exposure-response modelling (detailed below) suggests continued dosing up to 24 weeks will meet muscle and skin efficacy requirements in the DM TPP for TIS, CFB MMT-8, and CDASI-A response.

### Skin Cohort

The mean (SD) baseline CDASI-A scores in Stage 1, Stage 2, and Amended Stage 2 were 32.7 (10.6), 30.8 (9.4), and 33.3 (11.3) respectively (Table 8 and Table 9). In the pooled skin cohort at Week 12, the estimated CFB in CDASI-A scores of participants receiving 600 mg PF-06823859, 150 mg PF-06823859, and placebo in the first 12 weeks and the placebo-adjusted difference are presented in Table 11. Both doses of PF-06823859 differentiated from placebo at Week 12 with p<0.0001. Separation from placebo occurred as early as Week 4, with no plateau by Week 12. The estimated differences of the means in CFB (CDASI-A) at Week 12 presented in the Table 11 are numerically larger than the estimate (−8.2 (−11.3, −5.3)) of the treatment effect for CDASI-A observed in the ProDERM study.

TABLE 11
Statistical Analysis of Change from Baseline for CDASI Activity Total score
at Week 12 (Lancova-P, Pooled FAS for skin cohort {PFASS})

| | | | Difference |
|---|---|---|---|
| | | | vs. Placebo |
| | Treatment during first 12 Weeks | LS Mean (90% CI) | (90% CI) |
| CDASI-A | Placebo (N = 14) | −2.9 (−6.3, 0.45) | NA |
| | PF-06823859 150 mg (N = 15) | −16.6 (−19.8, −13.4) | −13.7 (−18.3, −9.0) |
| | PF-06823859 600 mg (N = 28) | −19.2 (−21.5, −16.8) | −16.2 (−20.4, −12.1) |

The sensitivity analysis for CDASI-A was conducted after removal of one subject on the 600 mg treatment arm in Stage 2 who took prohibited medications The sensitivity analysis is aligned with the primary analysis for mean CFB in CDASI-A.

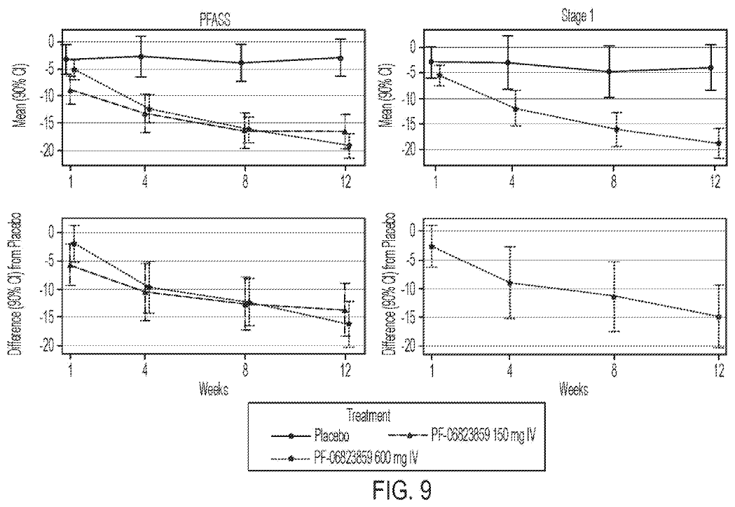

FIG. 9 shows that the estimates for the placebo and 600 mg dose in the pooled sample are similar to the estimates based on the Stage 1 data. The efficacy estimates for the 150 mg and 600 mg treatment arms based on the pooled data are nearly identical.

Supporting analyses on CDASI-A using responder rates (Table 12) and analysis of percentage change from baseline (Table 13) are aligned with the primary analysis and both doses of PF-06823859 differentiated from placebo. A change in CDASI-A score of 4 or 5 points represents a minimal clinically significant change (Anyanwu et al, 2015) and PF-06823859 achieved this decrease in CDASI-A score in more than 96% of the subjects. For patients with CDASI-A score >14 points, a 40% change in the CDASI-A score indicates a meaningful change in Quality of Life (Ahmed et al, 2020) and PF-06823859 achieved this decrease in CDASI-A score in more than 80% of the subjects.

TABLE 12
Summary of Responder rates of PF-06823859 on CDASI Activity Scores

| | | Percent of | Percent of |
|---|---|---|---|
| | Percent of | Responders | Responders |
| | Responders | (with 90% CI) | (with 90% CI) |
| | (with 90% CI) | in PF-06823859 | in PF-06823859 |
| | in Placebo | 150 mg IV Group | 600 mg IV |
| | Group (N = 14) | (N = 15) | Group (N = 28) |
| Definition of Responder | | | |
| Decrease of CDASI-A by >5 | 35.7 (16.3, 59.4) | 100.0 (84.6, 100.0) | 96.4 (84.9, 99.6) |
| Decrease of CDASI-A by >40% | 7.1 (0.7, 26.4) | 80.0 (57.7, 92.4) | 82.1 (66.7, 91.1) |

At week 12, the mean % CFB in CDASI-A for 150 mg and 600 mg of PF-06823859 compared to placebo was −44% and −50% respectively (compared to −35% at week 24 in the current Target Product Profile).

TABLE 13
Statistical Analysis of Percent Change from Baseline for CDASI Activity Total
Score at Week 12 (LANCOVA-P, Pooled FAS for Skin Cohort {PFASS})

| | Treatment during first 12 Weeks | LS Mean (90% CI) | Difference vs. Placebo (90% CI) |
|---|---|---|---|
| CDASI-A | Placebo (N = 14) | −9.5 (−18.6, −0.5) | NA |
| | PF-06823859 150 mg (N = 15) | −53.0 (−61.4, −44.6) | −43.5 (−55.8, −31.1) |
| | PF-06823859 600 mg (N = 28) | −59.0 (−65.3, −52.8) | −49.5 (−60.5, −38.5) |

Overall, both doses of PF-06823859 (600 mg and 150 mg) met the primary efficacy endpoint for the skin cohort as assessed by CDASI-A score. Although the study was not powered for efficacy evaluation for the muscle cohort, PF-06823859 600 mg is numerically better than placebo across all key muscle function endpoints (TIS, MMT-8, PtGA of myositis and CK) with nominal statistical significance for PtGA and CK. Sensitivity analyses excluding a single time point for a placebo subject who received prohibited concomitant medications reached nominal statistical significance in TIS, CK and PtGA. The 12-week efficacy results on CDASI-A are better and the results on muscle-related endpoints (i.e., TIS, MMT-8, PtGA, CK) are comparable or numerically better than the efficacy results from the only approved treatment for DM (ProDERM study).

### Pharmacokinetics

There was an approximate 4- and 3.2-fold increase in AUC and Cmax, respectively in DM patients with predominantly skin disease receiving 150 or 600 mg Q4W (Table 14). The PK of PF-06823859 in skin predominant DM and muscle predominant DM was similar and consistent with the exposures in the Phase 1 healthy participants (Table 14). PK data were only available for 2 Stage 3 participants for the Table 11 analysis.

TABLE 14
First-dose Pharmacokinetic Derived Parameter Estimates
of PF-06823859 Exposure in DM and Healthy Participants

| | | | Geo Mean | Geo Mean |
|---|---|---|---|---|
| Subject | | | AUC(0-28 d) μg · | Cmax μg/mL |
| Type | Dose mg | N | hr/mL (% CV) | (% CV) |
| DM (Skin) | 150 | 10 | 13293 (25.9) | 54.1 (29.0) |
| DM (Skin) | 600 | 28 | 45535 (28.9) | 173 (35.2) |
| DM (muscle) | 600 | 2 | 34033 (6.9) | 139 (47.4) |
| Healthy | 600 | 10 | 47094 (16.2) | 235 (26.1) |
| Subjects | | | | |

Assessment of Mechanistic Biomarker: Percent Change from Baseline in IP-10

IP-10 decreased over time with comparable mean percent reduction in IP-10 at Week 12 (pooled analysis) between skin predominant DM patients receiving either PF-06823859 150 mg (n=14; −70.25%) or 600 mg (n=27; −67.58%). The mean percent reduction in IP-10 for the Stage 3 participants who received active then placebo (n=9) was −38.89%. The model predicted median IP-10 levels of 624.0 pg/mL (90% prediction interval 507.2, 763.6) at baseline for the skin and muscle predominant DM participants, which decreased to 298.7 (255.2, 349.1) and 255.9 (220.0, 304.2) pg/mL at Week 12 for the 150 and 600 mg PF-06823859 doses, respectively. There was no difference between skin and muscle predominant subjects that could be identified in the model.

### Exposure-Response

A preliminary model was developed to predict clinical endpoints in response to inhibition of IFNb predicted from the PKPD model. Clinical trial simulations involving participants from all stages were used to predict CDASI-A and TIS response after 24-weeks of continued treatment with PF-06823859 or placebo (Table 15). A key limitation of the model is the assumption that all the endpoints have the same onset of drug effect. This assumption was necessary given limitation in the small sample size especially for the muscle cohort. The preliminary model predicts that non-TIS endpoints like CDASI-A will plateau at 12 weeks, and 14 weeks for TIS endpoints. Endpoint results are based on 300 trials in 100 participants (50 in active, 50 in placebo). TIS and MMT-8 results were based on Stage 3 demographics and CDASI-A results were based on earlier stages participants. Point estimates from the model are slightly different from but are generally consistent with observed summary responses.

TABLE 15
Predicted Response After 24-week of Continued Treatment

| Predicted Endpoint | 600 mg Q4 W | Placebo | Active - Placebo | TPP Achieved^{a} |
|---|---|---|---|---|
| Total Improvement | 53.7 (48.7, 58.9) | 28.1 (23.7, 32.7) | 25.5 (21.4, 30.7) | N/A |
| Score (absolute) | | | | |
| TIS Moderate | 78.7 (68.6, 86.8) | 23.9 (14.0, 35.5) | 54.2 (43.4, 64.7) | Yes |
| Responders (%) | | | | |
| MMT-8 Change from | 19.3 (14.2, 23.8) | 10.3 (5.8, 14.6) | 8.9 (4.6, 13.1) | Yes |
| Baseline | | | | |
| CDASI-A | 14.3 (11.8, 16.7) | 29.8 (26.6, 33.4) | −15.8 (−18.8, −12.7) | N/A |
| (absolute) | | | | |
| CDASI-A | −51.6 (−59.8, −43.1) | 3.2 (−3.9, 11.8) | −54.5 (−71.5, −41.9) | N/A |
| Percent Change from | | | | |
| Baseline (%) | | | | |
| CDASI-A | 66.3 (56.6, 76.3) | 5.0 (1.9, 8.9) | 61.2 (50.0, 72.5) | Yes |
| Responders ^{b} (%) | | | | |

Results are the median mean (or proportion) and 90% prediction interval of the estimate.
^{a}Based on 90% prediction intervals including or exceeding TPP-specified thresholds.
^{b} Proportion of subjects with percent changes from baseline less than −40% (i.e., 40% improvement)

### Immunogenicity

ADAs were reported for 3 of 22 (13.6%) and 1 of 5 (20%) in Stage 1 (600 mg IV) and Stage 2 (150 mg), respectively, from patients with skin predominant skin disease. No ADA were reported in any of the other stages. The low incidence of drug induced ADAs (3/22; Stage 1-600 mg) implies lack of clinically relevant impact on safety/PK/PD or efficacy. Exploratory models of the effect of ADA on PKPD parameters also did not suggest any impact. There were no events of anaphylaxis or immunologically related clinical responses of concern reported.

### Safety

The majority of TEAEs were mild and no dose relationship was observed. There was numerically higher number of TEAEs in the 150 mg group than the 600 mg group and placebo. The most common TEAEs by SOC in the study were: Infections and Infestations. The 600 mg dose group and Placebo had numerically higher incidence (27.7% and 20%, respectively) of Infections and Infestations compared to the 150 mg dose group (11.8%). There were 3 mild cases of infection in PF-06823859 groups that were treatment related compared to 2 cases in the placebo groups across all stages. There were 4 reports of SARS-CoV-2 test positive cases in placebo group and 1 suspected COVID-19 in PF-06823859 600 mg group. There were no cases of Herpes Zoster or Herpes Simplex in the study.

Additional common TEAEs by SOC (Table 16) were Investigations (600 mg group=19.1%, 150 mg group=11.8%, placebo group=22.2%), Nervous System Disorders (600 mg group=14.9%, 150 mg group=23.5%, placebo group=13.3%), Gastrointestinal disorders (600 mg group=12.8%, 150 mg group=11.8%, placebo group=17.8%, and Skin and subcutaneous tissue disorders (600 mg group=14.9%, 150 mg group=23.5%, placebo group=13.3%).

### Pharmacokinetics/Pharmacodynamics

The population PKPD model was developed based on the available PF-06823859 serum concentrations, IFNb levels, IP-10 levels, and gene signature from lesional/non-lesional skin and blood from 00251001 (where applicable) and C0251002. The most common (>5%) TEAEs by PT (Preferred Term) were Headache (N=3, 17.6%), and Arthralgia (N=2, 11.8%) for the 150 mg group (single case for all other PT). The most common PT for the 600 mg group were Headache (N=6, 12.8%), and Upper respiratory tract infection (N=4, 8.5%). The most common PT for the placebo group were Headache (N=5, 11.1%), Pruritus (N=5, 11.1%), SARS-CoV-2 test positive (N=4, 8.9%), Fatigue (N=3, 6.7%), and Sinusitis (N=3, 6.7%).

TABLE 16
Treatment-Emergent Adverse Events by System Organ Class
(All Causalities) (All Stages***) (Week 0-Week 24)

| | Number of Participants Evaluable for AEs | | | |
|---|---|---|---|---|
| | Placebo | PF-06823859 150 | PF-06823859 600 | Total* |
| | (N = 45) | mg IV (N = 17) | mg IV (N = 47) | (N = 75) |
| | Number (%) of Participants**: by SYSTEM ORGAN CLASS | | | |
| | n (%) | n (%) | n (%) | n (%) |
| With Any adverse event | 30 (66.7) | 13 (76.5) | 33 (70.2) | 61 (81.3) |
| BLOOD AND LYMPHATIC SYSTEM | 1 (2.2) | 1 (5.9) | 3 (6.4) | 4 (5.3) |
| DISORDERS | | | | |
| CARDIAC DISORDERS | 1 (2.2) | 1 (5.9) | 1 (2.1) | 2 (2.7) |
| EAR AND LABYRINTH DISORDERS | 1 (2.2) | 0 | 1 (2.1) | 2 (2.7) |
| EYE DISORDERS | 3 (6.7) | 0 | 1 (2.1) | 4 (5.3) |
| GASTROINTESTINAL DISORDERS | 8 (17.8) | 2 (11.8) | 6 (12.8) | 16 (21.3) |
| GENERAL DISORDERS AND | 6 (13.3) | 1 (5.9) | 5 (10.6) | 12 (16.0) |
| ADMINISTRATION SITE CONDITIONS | | | | |
| HEPATOBILIARY DISORDERS | 0 | 0 | 1 (2.1) | 1 (1.3) |
| IMMUNE SYSTEM DISORDERS | 1 (2.2) | 0 | 0 | 1 (1.3) |
| INFECTIONS AND INFESTATIONS | 9 (20) | 2 (11.8) | 13 (27.7) | 24 (32.0) |
| INJURY, POISONING AND PROCEDURAL | 4 (8.9) | 3 (17.6) | 5 (10.6) | 8 (10.7) |
| COMPLICATIONS | | | | |
| INVESTIGATIONS | 10 (22.2) | 2 (11.8) | 9 (19.1) | 17 (22.7) |
| METABOLISM AND NUTRITION DISORDERS | 1 (2.2) | 0 | 1 (2.1) | 2 (2.7) |
| MUSCULOSKELETAL AND CONNECTIVE | 3 (6.7) | 3 (17.0) | 8 (17.0) | 12 (16.0) |
| TISSUE DISORDERS | | | | |
| NEOPLASMS BENIGN, MALIGNANT AND | 0 | 1 (5.9) | 1 (2.1) | 2 (2.7) |
| UNSPECIFIED (INCL CYSTS AND POLYPS) | | | | |
| NERVOUS SYSTEM DISORDERS | 6 (13.3) | 4 (23.5) | 7 (14.9) | 17 (22.7) |
| PSYCHIATRIC DISORDERS | 2 (4.4) | 0 | 1 (2.1) | 3 (4.0) |
| RENAL AND URINARY DISORDERS | 0 | 0 | 1 (2.1) | 1 (1.3) |
| REPRODUCTIVE SYSTEM AND BREAST | 0 | 1 (5.9) | 0 | 1 (1.3) |
| DISORDERS | | | | |
| RESPIRATORY, THORACIC AND | 4 (8.9) | 1 (5.9) | 6 (12.8) | 10 (13.3) |
| MEDIASTINAL DISORDERS | | | | |
| SKIN AND SUBCUTANEOUS TISSUE | 9 (20.0) | 4 (23.5) | 2 (4.3) | 13 (17.3) |
| DISORDERS | | | | |
| SURGICAL AND MEDICAL PROCEDURES | 1 (2.2) | 0 | 0 | 1 (1.3) |
| VASCULAR DISORDERS | 0 | 0 | 2 (4.3) | 2 (2.7) |

*Totals for the No. of participants at a higher level were not necessarily the sum of those at the lower levels since a participant may report two or more different adverse events within the higher-level category.
**Participants were only counted once per treatment per event.
***Included all data collected since the first dose of study

There were 2 SAEs reported in two participants receiving 600 mg of PF-06823859, and 1 SAE in a participant receiving placebo in Stage 1. There were no SAEs among participants receiving 600 mg PF-06823859, 150 mg PF-06823859, and placebo respectively in Stage 2. In Amended Stage 2, there were no SAEs among those receiving either 600 mg or 150 mg PF-06823859 or placebo during the first 12 weeks. In Stage 3, there was 2 SAE among those receiving 600 mg of PF-06823859 during the first 12 weeks and no SAEs among those receiving placebo during the first 12 weeks.

There were no clinically significant changes from baseline for safety labs, ECG, and vital signs after treatment with PF-06823859 150 mg and 600 mg doses.

Overall, in the skin cohort of the study (Stage 1, Stage 2, and Amended Stage 2) 150 mg (Total N=10), and 600 mg (Total N=25) PF-06823859 administered once monthly for a total of 3 doses were generally well tolerated and safe in patients with DM.

In the muscle cohort (total N=9) in stage 3, 600 mg PF-06823859 administered once monthly for a total of 3 doses were also generally well tolerated and safe in patients with DM.

### Example 4

### Support for Potential Efficacy of Anti-IFNß in Systemic Lupus Erythematosus (SLE), Cutaneous Lupus, Lupus Nephritis and Other Select Diseases.

To assess the potential of our anti-IFNß antibody, we analyzed up to 3 well-powered transcriptomics datasets in each of the following indications: Atopic Dermatitis (AD), Alopecia, Cachexia, Crohn's Disease, Cutaneous Lupus, Idiopathic Pulmonary Fibrosis, NASH, Ulcerative Colitis, Psoriasis, Rheumatoid Arthritis, Systemic Lupus Erythematosus (SLE), Scleroderma, and Vitiligo. Studies differ in the collected tissue and number of patient samples. A total of 3880 samples of lesional and non-lesional tissue as well as patients and healthy control subjects (for comparison) were analyzed. Detailed information for each dataset is shown in Table 10.

Indication data was processed by the company CytoReason according to best practices and provided as normalized per-sample expression values. In the Dermatomyositis trial of the anti-IFNß antibody (PF-06823859), a specific type-1 IFN signature was monitored. The signature is defined based on the average gene expression (log₂CPM, Counts per million reads) of the following 10 genes suggested by Wong et al., 2012: “RSAD2”, “IFIT1”, “IF144L”, “IF127”, “IF144”, “CXCL10”, “IF16”, “ISG15”, “CMPK2”, “HERC5”.

For each indication, we computed this per-sample type-1 IFN signature. We then applied a one-sided Wilcoxon statistical test to assess differences between lesional and non-lesional tissue or disease vs healthy tissues. Details for each comparison are in Table 17. We also applied the same method to our internal clinical trial data of the anti-IFNß antibody (PF-06823859) in dermatomyositis patients.

As a total of 50 comparisons were tested, a threshold of 0.001 is considered significant after multiple testing correction. To assess the strength of the difference in type-1 IFN signature, effects were estimated as the medians of the difference between the lesional and non-lesional or disease vs healthy tissues, respectively. The confidence interval is obtained through a Hodges-Lehmann estimator or normal approximations depending on the availability of exact p-values.

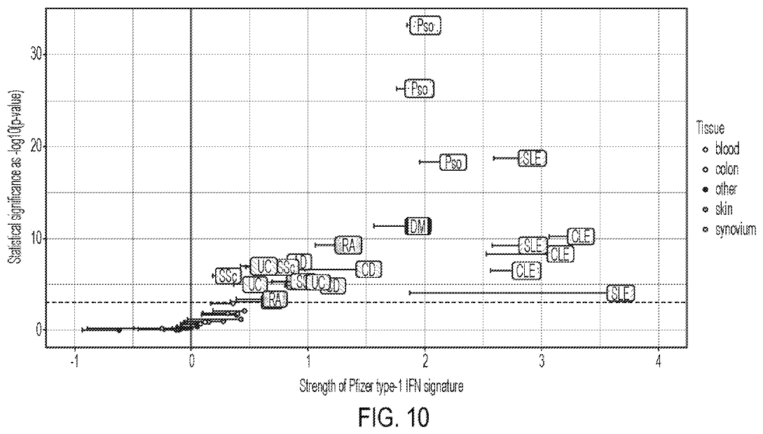

FIG. 10 highlights all studies with significant differences based on the 10-gene Type-1 INF signature. In particular, Systemic Lupus Erythematosus (SLE), Cutaneous Lupus, and Psoriasis show significant differences on par with the differences observed in the internal anti-IFNß Dermatomyositis trial.

Other indications that show significant type-1 IFN signature differences based on the signature are Ulcerative Colitis, Crohn's Disease, Rheumatoid Arthritis, Atopic Dermatitis and Scleroderma.

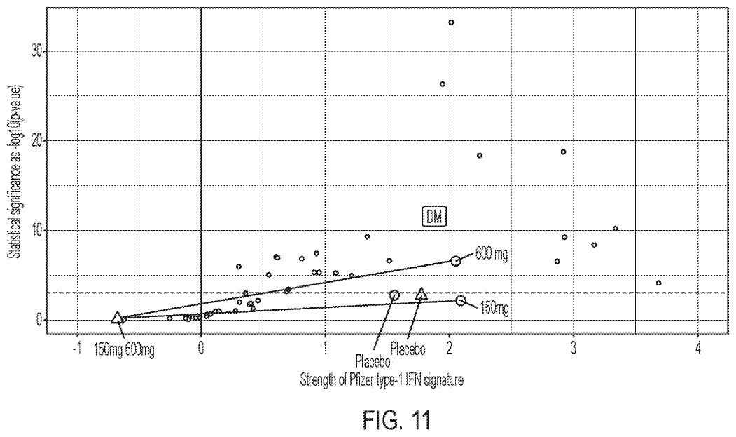

FIG. 11 shows the effect of our anti-IFNß antibody on the type-1 IFN signature in context of all other assessed indications and their differential type-1 IFN signature signal. For each treatment arm in our Dermatomyositis trial (150 mg dose, 600 mg dose, and placebo), we evaluate the type-1 IFN signature at baseline (circles) and post-treatment (triangles) in lesional skin samples in contrast to baseline non-lesional skin samples. Placebo treatment leads to minimal change in the lesional type-1 IFN signature. In contrast, the 150 mg as well as the 600 mg dose ablate the signature in lesional skin to at or below the level of non-lesional skin at baseline.

In summary, these data support the potential for the anti-IFNß antibody PF-06823859 in additional indications highlighting SLE, Cutaneous Lupus, and Psoriasis, and showing precision medicine potential for Ulcerative Colitis, Crohn's Disease, Rheumatoid Arthritis, Atopic Dermatitis and Scleroderma.

Similar results were found when these data were repeated with a list of 13 type I IFN-inducible genes: (IF144L, IF144, IFIT1, IFIT3, RSAD2, ISG15, HERC5, IF16, OAS3, MX1, EPST11, CXCL10, and CMPK2) as disclosed in Greenberg et al 2012 and Tabata et al 2023.

TABLE 17
Details of each comparison made between lesional and non-lesional
tissue or disease vs healthy tissues from various of indications.

| comparison | ds_id | cases | Controls | tissue |
|---|---|---|---|---|
| alopecia L/NL | GSE148346 | 15 | 15 | skin |
| alopecia L/NL | GSE68801_batch2013 | 18 | 18 | skin |
| AD | GSE133477 | 79 | 40 | skin |
| AD | GSE157194 | 54 | 54 | skin |
| AD | GSE99802 | 53 | 53 | skin |
| cachexia | GSE130563 | 24 | 19 | other |
| cachexia | GSE133979 | 17 | 11 | other |
| cachexia | GSE41726 | 47 | 70 | other |
| CD | GSE52746 | 10 | 17 | colon |
| CD | GSE83687 | 11 | 45 | colon |
| CD | GSE95437 | 20 | 29 | colon |
| CD | GSE24287 | 40 | 25 | other |
| CLE | GSE109248 | 25 | 14 | skin |
| CLE | GSE112943 | 15 | 10 | skin |
| CLE | GSE81071 | 90 | 13 | skin |
| IPF | GSE110147 | 22 | 11 | other |
| IPF | GSE124685 | 49 | 35 | other |
| IPF | GSE32537 | 75 | 49 | other |
| NASH vs NAFLD | E-MTAB-4856-GPL16022_Italy | 15 | 31 | other |
| NASH vs NAFLD | GSE135251 | 153 | 51 | other |
| NASH vs NAFLD | PRJNA512027 | 103 | 27 | other |
| Pediatric UC | GSE10616 | 10 | 16 | colon |
| Pso | GSE117239 | 83 | 84 | skin |
| Pso | GSE117468 | 126 | 126 | skin |
| Pso | GSE13355 | 57 | 56 | skin |
| RA | GSE110169 | 84 | 77 | blood |
| RA | GSE45291 | 90 | 20 | blood |
| RA | GSE55235 | 10 | 10 | synovium |
| RA | GSE55457 | 13 | 10 | synovium |
| RA | GSE89408 | 147 | 28 | synovium |
| SLE | GSE110169 | 73 | 71 | blood |
| SLE | GSE110174 | 140 | 10 | blood |
| SLE | GSE49454 | 157 | 20 | blood |
| SSc L/NL | GSE45485 | 12 | 12 | skin |
| SSc L/NL | GSE9285 | 19 | 19 | skin |
| SSc | GSE130955 | 58 | 33 | skin |
| SSc | GSE58095 | 63 | 36 | skin |
| SSc | GSE95065 | 18 | 15 | skin |
| UC | GSE112057 | 15 | 12 | blood |
| UC | GSE94648 | 19 | 22 | blood |
| UC | GSE73661 | 67 | 12 | colon |
| UC | GSE83687 | 27 | 45 | colon |
| UC | GSE92415 | 87 | 21 | colon |
| vitiligo L/NL | GSE65127 | 10 | 10 | skin |
| DM_PBO_Baseline | internal | 9 | 9 | skin |
| DM_150 mg_Baseline | internal | 7 | 7 | skin |
| DM_600 mg_Baseline | internal | 24 | 24 | skin |
| DM_PBO_Treated | internal | 9 | 9 | skin |
| DM_150 mg_Treated | internal | 7 | 7 | skin |
| DM_600 mg_Treated | internal | 23 | 23 | skin |

### Example 5. Developability Study of IFNß-PF Formulation at 150 mg/1 mL or Higher Concentration

Current Drug Product (DP) for Anti-IFNß PF-06823859 is formulated at 100 mg/mL in 20 mM histidine, 85 mg/mL sucrose, 0.05 mg/mL EDTA, 0.2 mg/mL polysorbate 80 at pH 5.8. High concentration formulation development is planned to ease drug delivery to patients as well as to allow flexibility for accommodating both intravenous (IV) and subcutaneous (SC) administration. This study evaluated the developability of PF-06823859 (IFNß) formulation at 150 mg/mL and focused on strategies to reduce viscosity at higher concentrations (>150 mg/ml).

The impact to product viscosity and osmolality was assessed by varying pH, protein concentration, arginine, sucrose, and sodium chloride. Additionally, viscosity of drug substance in histidine buffer and in full formulation at different protein concentrations was compared.

### Effect of pH

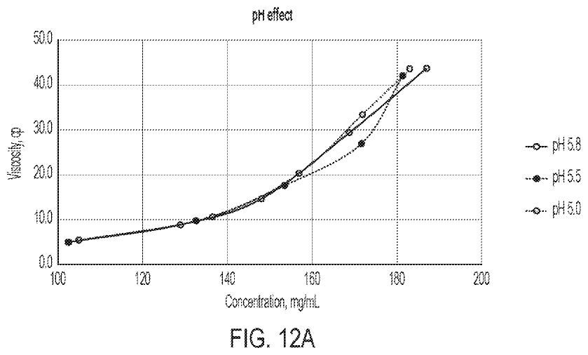

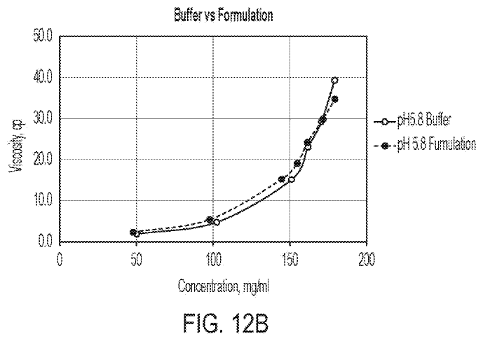

To test the effect of pH and concentration, anti-IFNß (PF-06823859) samples were prepared in 20 mM histidine buffer at pH 5.8, 5.5, and 5.0 (no excipients were added). Original anti-IFNß (PF-06823859) drug substance (in 20 mM histidine buffer, pH 5.8) was buffer exchanged to target pH value and then concentrated to ~190 mg/mL followed by dilution using 20 mM histidine buffer at targeted pH. Prepared samples were tested for viscosity and concentration and results are plotted in FIG. 12A. Results show that varying the pH from 5.0 to 5.8 at concentrations from 100 mg/ml to 190 mg/ml did not significantly impact the viscosity of the samples.

To evaluate if the excipients (sucrose, PS80, and EDTA) affect viscosity, anti-IFNß (PF-06823859) viscosity in 20 mM histidine buffer (pH 5.8) was compared to anti-IFNß (PF-06823859) in formulation (20 mM histidine buffer, 85 mg/mi sucrose, 0.2 mg/ml PS80, and 0.05 mg/ml EDTA). Test results did not show a significant difference in viscosity between buffer and full formulation (FIG. 12B). The raw data used for FIG. 12A and FIG. 12B is shown in Table 18.

TABLE 18
Tested Results

| | 20 mM | | | | | | | | | |
|---|---|---|---|---|---|---|---|---|---|---|
| | His | Designed Formulation | | | | | Tested Results | | | |
| Samples ID | buffer | A | S | T | N | P | p | C | V | O |
| 00705945-0495-M03 | pH 5.8 | | | | | | 5.8 | 52 | 1.9 | |
| 00705945-0495-M04 | buffer | | | | | | 5.8 | 103 | 5.0 | |
| 00705945-0495-M05 | | | | | | | 5.8 | 129 | 8.8 | |
| 00705945-0495-M06 | | | | | | | 5.8 | 148 | 14.6 | |
| 00705945-0495-M07 | | | | | | | 5.8 | 169 | 29.4 | |
| 00705945-0495-M08 | | | | | | | 5.8 | 187 | 43.7 | |
| 00706600-0241-M01 | pH 5.5 | | | | | | 5.6 | 102 | 5.0 | |
| 00706600-0241-M02 | buffer | | | | | | 5.7 | 133 | 9.7 | |
| 00706600-0241-M03 | | | | | | | 5.8 | 154 | 20.3/17.2* | |
| 00706600-0241-M04 | | | | | | | 5.8 | 172 | 26.9 | |
| 00706600-0241-M05 | | | | | | | 5.8 | 181 | 30.8/42.0* | |
| 00706600-0241-M06 | | | | | | | 5.8 | 198 | 46.9 | |
| 00706600-0241-M07 | pH 5.0 | | | | | | 5.1 | 105 | 5.4 | |
| 00706600-0241-M08 | buffer | | | | | | 5.2 | 137 | 10.6 | |
| 00706600-0241-M09 | | | | | | | 5.2 | 157 | 20.3/19.6* | |
| 00706600-0241-M10 | | | | | | | 5.2 | 172 | 33.4 | |
| 00706600-0241-M11 | | | | | | | 5.2 | 183 | 43.6/40.8* | |
| 00706600-0241-M12 | | | | | | | 5.2 | 203 | 42.7 | |
| 00706600-0229-M01 | pH 5.8 | | | | | | 5.8** | 50 | 1.9 | |
| | buffer | | | | | | | | | |
| 00706600-0229-M02 | | | | | | | 5.8** | 102 | 4.8 | |
| 00706600-0229-M03 | | | | | | | 5.8** | 151 | 15.2 | |
| 00706600-0229-M04 | | | | | | | 5.8** | 162 | 23.1 | |
| 00706600-0229-M05 | | | | | | | 5.8** | 171 | 29.3 | |
| 00706600-0229-M06 | | | | | | | 5.8** | 179 | 39.3 | |
| 00706600-0229-M07 | | | | | | | 5.8** | 186 | 48.6 | |
| 00706600-0229-M08 | pH 5.8 | | 85 | | | 0.2/0.05 | 5.8** | 48 | 2.4 | |
| 00706600-0229-M09 | formulation | | 85 | | | 0.2/0.05 | 5.8** | 98 | 5.4 | |
| 00706600-0229-M10 | (with | | 85 | | | 0.2/0.05 | 5.8** | 145 | 15.3 | |
| 00706600-0229-M11 | sucrose, | | 85 | | | 0.2/0.05 | 5.8** | 155 | 19.1 | |
| 00706600-0229-M12 | pH 80, | | 85 | | | 0.2/0.05 | 5.8** | 162 | 24.2 | |
| 00706600-0229-M13 | and | | 85 | | | 0.2/0.05 | 5.8** | 172 | 29.8 | |
| 00706600-0229-M14 | EDTA) | | 85 | | | 0.2/0.05 | 5.8** | 179 | 34.7 | |
| 00706600-0229-M15 | | | 85 | | | 0.2/0.05 | 5.8** | 187 | 45.4 | |
| 00706600-0241-M19 | pH 5.8 | 150 | 85 | | | | 5.9 | 145 | 13.5 | 729 |
| | w/argi | | | | | | | | | |
| | and | | | | | | | | | |
| | sucr | | | | | | | | | |
| 00706600-0241-M20 | | 180 | 85 | | | | 6.0 | 170 | 23.3 | 753 |
| 00706600-0241-M21 | pH 5.5 | 150 | 85 | | | | 5.8 | 144 | 13.5 | 711 |
| | w/argi | | | | | | | | | |
| | and | | | | | | | | | |
| | sucr | | | | | | | | | |
| 00706600-0241-M22 | pH 5.0 | 150 | 85 | | | | 5.3 | 146 | 14.2 | 673 |
| 00706600-0241-M23 | w/argi | 180 | 85 | | | | 5.3 | 171 | 25.1 | 744 |
| | and | | | | | | | | | |
| | treha | | | | | | | | | |
| 00706600-0243-M07 | pH 5.8, | 100 | 50 | | | | 5.9 | 155 | 13.7 | 503 |
| 00706600-0243-M08 | 0-100 mM | 50 | 50 | | | | 5.9 | 154 | 15.4 | 393 |
| 00706600-0243-M09 | Argi & | 0 | 50 | | | | 5.9 | 154 | 17.9 | 289 |
| | 5% | | | | | | | | | |
| | Sucr | | | | | | | | | |
| 00706600-0243-M10 | pH 5.0, | 100 | | 50 | | | 5.2 | 153 | 13.7 | 487 |
| 00706600-0243-M11 | 50-100 | 50 | | 50 | | | 5.2 | 154 | 14.4 | 400 |
| | mM | | | | | | | | | |
| | Argi & | | | | | | | | | |
| | 5% | | | | | | | | | |
| | Treh | | | | | | | | | |
| 00715692-0022-M01 | pH 6.0, | | 85 | | | 0.2/0.05 | 6.1 | 152.9 | 19.4 | 428 |
| | 85 mg/ml | | | | | | | | | |
| | Sucrose | | | | | | | | | |
| 00715692-0022-M02 | pH 6.0, | 50 | 50 | | | 0.2/0.05 | 6.1 | 151.2 | 12.8 | 363 |
| | 50 mg/ml | | | | | | | | | |
| | sucrose, | | | | | | | | | |
| | 50 mM | | | | | | | | | |
| | Arginine | | | | | | | | | |
| 00715692-0022-M03 | pH 6.0, | | 50 | | 50 | 0.2/0.05 | 6.2 | 151.1 | 14.2 | 375 |
| | 50 mg/ml | | | | | | | | | |
| | sucrose, | | | | | | | | | |
| | 50 mM | | | | | | | | | |
| | NaCl | | | | | | | | | |
| 00715692-0022-M04 | pH 6.5, | | 85 | | | 0.2/0.05 | 6.6 | 147.9 | 21.6 | 405 |
| | 85 mg/ml | | | | | | | | | |
| | Sucrose | | | | | | | | | |
| 00715692-0022-M05 | pH 6.5, | 50 | 50 | | | 0.2/0.05 | 6.6 | 147.9 | 12.7 | 371 |
| | 50 mg/ml | | | | | | | | | |
| | Sucrose, | | | | | | | | | |
| | 50 mM | | | | | | | | | |
| | Arginine | | | | | | | | | |
| 00715692-0022-M06 | pH 6.5, | | 50 | | 50 | 0.2/0.05 | 6.7 | 151 | 15.2 | 390 |
| | 50 mg/ml | | | | | | | | | |
| | Sucrose, | | | | | | | | | |
| | 50 mM | | | | | | | | | |
| | NaCl | | | | | | | | | |

*repeat test.
**designed pH value.
Abbreviations: A = Argi; M. S = Sucr; mg/ml; T = Treh, mg/ml; N = NaCl; mM; P = PS80/EDTA; p = pH; C = Conc, g/ml; V = Viscosity, p; O = Osmo, Osmo.

### Effect of Arginine on Viscosity

To minimize the viscosity of formulations at higher concentrations, arginine (150 mM) was added to anti-IFNß (PF-06823859) in 20 mM histidine buffer with 85 mg/ml sucrose (at pH 5.8 and 5.5) and with 85 mg/mL trehalose (at pH 5.0). Trehalose was selected for the pH 5.0 sample was to avoid sucrose hydrolysis at the lower pH. The data in Table 18 shows that the viscosities of all 150 mg/ml samples at pH 5.8, 5.5, and 5.0 were 13.5 cp, 13.5 cp, and 14.2 cp, respectively. Viscosity of 180 mg/ml samples with 150 mM arginine were 23.3 cp and 25.2 cp, at pH 5.8 and 5.0, respectively. This data indicated that viscosity was not reduced by lowering the pH of the samples. However, 150 mM arginine significantly reduced the viscosity to 23.3 cp (00706600-0241-M20) from the 29.8 cp of the sample that contained no arginine (00706600-0229-M13). It was noted that osmolality was tested higher than 700 mOsm for all samples except the M22 which was 673 mOsm.

TABLE 19
Effect of Arginine and pH on anti-IFNβ (PF-06823859) Viscosity

| | Designed Formulation | | | | | Tested Results | | | |
|---|---|---|---|---|---|---|---|---|---|
| | IFNb Conc, | | Argi, | Sucr, | Treh, | Conc, | | Viscosity, | Osmo, |
| Samples ID | mg/ml | pH | mM | mg/ml | mg/ml | mg/ml | pH | cp | mOsm |
| 00706600- | 150 | 5.8 | 150 | 85 | | 145 | 5.9 | 13.5 | 729 |
| 0241-M19 | | | | | | | | | |
| 00706600- | 150 | 5.5 | 150 | 85 | | 144 | 5.8 | 13.5 | 711 |
| 0241-M21 | | | | | | | | | |
| 00706600- | 150 | 5.0 | 150 | | 85 | 146 | 5.3 | 14.2 | 673 |
| 0241-M22 | | | | | | | | | |
| 00706600- | 180 | 5.8 | 150 | 85 | | 170 | 6.0 | 23.3 | 753 |
| 0241-M20 | | | | | | | | | |
| 00706600- | 180 | 5.0 | 150 | | 85 | 171 | 5.3 | 25.1 | 744 |
| 0241-M23 | | | | | | | | | |
| 00706600- | 180 | 5.8 | 0 | 85 | | 172 | 5.8* | 29.8 | NT |
| 0229-M13 | | | | | | | | | |

*this was the designed pH

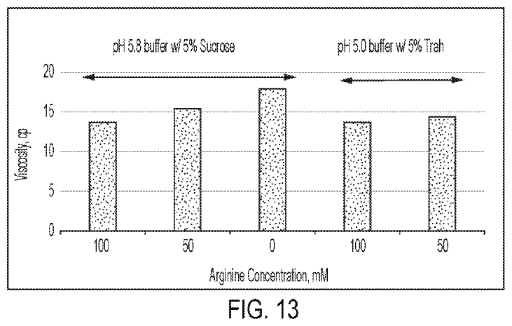

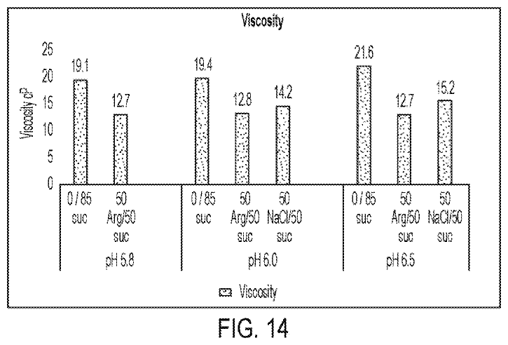

To make subcutaneous (SC) injection of high concentration anti-IFNß (PF-06823859) drug product in prefilled syringe a more feasible option, an osmolality value below 500 mOsm is preferred. To reduce the osmolality, additional tests were performed for 150 mg/ml anti-IFNß (PF-06823859) formulated in 20 mM histidine buffer with lower arginine concentrations (100 mM, 50 mM) and lower sugar concentration (50 mg/ml). Tested results are described in Table 19 and plotted in FIG. 13 and FIG. 14 shows that the viscosity increased when lowering the arginine concentration, therefore keeping the arginine level higher than 50 mM is preferred. The osmolality tested for all samples was 500 mOsm or lower, which met the study request.

TABLE 20
anti-IFNβ (PF-06823859) Viscosity with Lower Arginine and Sucrose Concentration

| | Designed Formulation | | | | | Tested Results | | | | | | |
|---|---|---|---|---|---|---|---|---|---|---|---|---|
| | Conc, | | Argi, | Sucr, | Treh, | Conc, | | Viscosity, | Osmo | | | |
| Samples ID | mg/ml | pH | mM | mg/ml | mg/ml | mg/ml | pH | cp | mOsm | Color | Clarity | Particulates |
| 00706600- | 150 | 5.8 | 100 | 50 | | 155 | 5.9 | 13.7 | 503 | ≤B9 | ≤Ref III | 1-5 |
| 0243-M07 | | | | | | | | | | | | |
| 00706600- | 150 | 5.8 | 50 | 50 | | 154 | 5.9 | 15.4 | 393 | ≤B9 | ≤Ref II | 1-5 |
| 0243-M08 | | | | | | | | | | | | |
| 00706600- | 150 | 5.8 | 0 | 50 | | 154 | 5.9 | 17.9 | 289 | ≤B9 | ≤Ref II | EFVP |
| 0243-M09 | | | | | | | | | | | | |
| 00706600- | 150 | 5.0 | 100 | | 50 | 153 | 5.2 | 13.7 | 487 | ≤B9 | ≤Ref II | 1-5 |
| 0243-M10 | | | | | | | | | | | | |
| 00706600- | 150 | 5.0 | 50 | | 50 | 154 | 5.2 | 14.4 | 400 | ≤B9 | ≤Ref III | TMTC |
| 0243-M11 | | | | | | | | | | | | |

### Effect of Sodium Chloride (NaCl) on IFNb Viscosity

To minimize the viscosity of formulations, NaCl (50 mM) was tested for anti-IFNß ~(PF-06823859) in 20 mM histidine buffer with 50 mg/mi sucrose (at pH 6.0 and 6.5). These samples were compared with samples containing 50 mM Arginine (pH 6.0 and 6.5) and samples containing neither Arginine nor NaCl (pH 60 and 6.5). All samples contained 150 mg/mi anti-IFNß (PF-06823859). Prepared samples were tested for viscosity, osmolality, concentration, and pH and the data is shown in Table 21.

FIG. 14 shows the comparison between using 50 mM Arginine, 50 mM NaCl, and without both at the pH of 6.0 and 6.5. This data shows that no advantage to viscosity by increasing the pH and Arginine has a greater ability to lower viscosity than NaCl in anti-IFNß (PF-06823859).

TABLE 21
anti-IFNβ (PF-06823859) Viscosity with
50 mM NaCl vs 50 mM Arginine at Different pH

| | Designed Formulation | | | | | Tested Results | | | | |
|---|---|---|---|---|---|---|---|---|---|---|
| | Conc, | | Arg, | Sucr, | NaCl, | | | Conc, | Viscosity, | Osmo |
| Sample IDs | mg/ml | pH | mM | mg/ml | mM | | pH | mg/ml | cp | mOsm |
| 00715692- | 150 | 6.0 | | 85 | | | 6.1 | 153 | 19.4 | 428 |
| 0022-M01 | | | | | | | | | | |
| 00715692- | 150 | 6.0 | 50 | 50 | | | 6.1 | 151 | 12.8 | 363 |
| 0022-M02 | | | | | | | | | | |
| 00715692- | 150 | 6.0 | | 50 | 50 | | 6.2 | 151 | 14.2 | 375 |
| 0022-M03 | | | | | | | | | | |
| 00715692- | 150 | 6.5 | | 85 | | | 6.6 | 148 | 21.6 | 405 |
| 0022-M04 | | | | | | | | | | |
| 00715692- | 150 | 6.5 | 50 | 50 | | | 6.6 | 148 | 12.7 | 371 |
| 0022-M05 | | | | | | | | | | |
| 00715692- | 150 | 6.5 | | 50 | 50 | | 6.7 | 151 | 15.2 | 390 |
| 0022-M06 | | | | | | | | | | |

This study evaluated the developability of the anti-IFNß (PF-06823859) formulation at 150 mg/mL or higher concentrations and investigated the effect of pH, salt, arginine, and sucrose on the viscosity and osmolality of the formulation. The data demonstrated that pH variation from 6.5 to 5.0 did not significantly impact viscosity of the anti-IFNß (PF-06823859) samples, and that both arginine and sodium chloride were comparable in their ability to reduce the viscosity of the formulations.

50-100 mM or higher arginine conferred satisfactory viscosity and osmolality levels to the formulation. Thus, the high concentration formulation proposed for further study is suggested as 150 mg/ml anti-IFNß (PF-06823859) in 20 mM histidine buffer containing 50-100 mM arginine and 50-85 mg/ml sucrose with 0.2 mg/ml polysorbate 80 (PS 80) and 0.05 mg/ml EDTA, pH 5.8.

### Example 6. Anti-IFNß (PF-06823859) 150 mg/mL Drug Product Formulation Nomination Study

To evaluate the anti-IFNß (PF-06823859) formulation, a stability study was conducted using anti-IFNß (PF-06823859) drug product at 150 mg/mL and varying amounts of arginine and sucrose in the formulation. In addition to evaluating the physical (aggregation, precipitation, denaturation) and chemical (oxidation) stability of anti-IFNß (PF-06823859), the study assessed formulation viscosity to identify the high concentration formulation most feasible to accommodate both IV and SC administrations. The formulations used in this study are provided in Table.

TABLE 22
anti-IFNβ (PF-06823859) Formulations

| Material ID | | Arginine | Sucrose | EDTA | PS80 | |
|---|---|---|---|---|---|---|
| (Description) | Histidine (mM) | (mM) | (mg/ml) | (mg/ml) | (mg/ml) | pH |
| M01(pH 5.8) | 20 | 100 | 50 | 0.05 | 0.2 | 5.8 |
| M02(pH 5.8) | 20 | 50 | 50 | 0.05 | 0.2 | 5.8 |
| M03(pH 5.8) | 20 | 50 | 85 | 0.05 | 0.2 | 5.8 |
| M04(pH 5.8) | 20 | 0 | 85 | 0.05 | 0.2 | 5.8 |

All formulations contained 150 mg/mL anti-IFNβ (PF-06823859).

The anti-IFNß (PF-06823859) samples were filtered through a 0.2 micron PES filter, filled with 1 mL drug product in 2 mL vials, stoppered, and stored at −20 °C, 5 °C, 25 °C, and 40 °C. Samples were tested according to the stability sample pulling schedule in 20. A subset of the samples was additionally subjected to freeze-thaw and agitation stress. Product quality attributes were assessed using the analytical methods listed in Table 23.

TABLE 23
Stability Study Time Points and Analytics

| | T0 | T2 wk | T4 | T8* | T12 |
|---|---|---|---|---|---|
| Temp | (May 10, | (May 24, | (Jun. 7, | (Jul. 5, | (Aug. 2, |
| (°C) | 2021) | 2021) | 2021) | 2021) | 2021) |
| 5 | A-N | A-J | A-J | A-J | A-J |
| 25 | | A-J | A-J | A-J | A-J |
| 40 | | A-J | A-J | A-J | A-J |
| −20 | | N/A | A-J | A-J | A-J |

*Decision time-point (formulation nomination will be based on 8 weeks data)
Freeze thaw: appearance, SVP, SEC, pH, and UV-Vis (only F/T cycle 5 samples will be tested, F/T Cycle 1, 3 will be kept at frozen condition

TABLE 24
Analytical Methods

| | Technique |
|---|---|
| A | Appearance |
| B | MFI (Micro Flow Imaging for SVP (Sub-visible particles) |
| C | HIAC (Ultra Low volume) for SVP(Sub-visible particles) |
| D | pH |
| E | Concentration (SoloVPE) |
| F | Size Exclusion Chromatography (SEC) |
| G | Imaged Capillary Isoelectric Focusing (iCE) |
| H | Nonreducing Capillary Gel Electrophoresis (nrCGE) |
| I | Reducing Capillary Gel Electrophoresis (rCGE) |
| J | Methionine Oxidation (MetOx) |
| K | Viscosity |
| L | Osmolality (Freezing Point Osmometer) |
| M | Differential Scanning Calorimetry (DSC) |
| N | Excipient testing (Arginine, Histidine and Sucrose) |
| | T0 and information Only |

No significant changes in appearance and pH were observed for all four formulations at all storage conditions. For concentration by UV-Vis, no trend was observed in all formulations; some increase in concentration was noted at 8 weeks, likely because the solution may not have been mixed well before the testing and values were back to normal after 12 weeks. For SEC data, there was no significant change in % monomer after 12 weeks at ~20 °C or 5 °C; there was a ~1% decrease at 25 °C and ~6% at 40 °C for all four formulations. The observed changes were expected for high concentration monoclonal antibodies at the 25 °C and 40 °C storage temperatures. Similarly for iCE, at 12 weeks of stability there was no significant change in % acidic species at −20 °C or 5 °C; an increase of ~5-6% at 25 °C and ~35-40% increase at 40 °C were observed. The trending was similar in all four formulations and the observed changes were expected for high concentration monoclonal antibodies at the storage conditions 25 and 40 °C. The nrCGE assay showed no significant downward trending for % IgG or upward trending of % fragments at −20 °C and 5 °C. An increase in fragments and decrease in IgG with increasing temperature was observed at 25 °C and 40 °C for most of the formulations tested, which is expected for high concentration monoclonal antibodies at higher temperatures. For rCGE, no clear trending in % IgG or % fragments was observed, and the trending was similar in all formulations tested in the study. Methionine oxidation data displayed an increasing trend in oxidation levels with storage temperature and time across all four formulations. For all formulations, there were no significant differences in T Onset values measured by DSC (Differential Scanning Calorimetry). Subvisible particulate counts were evaluated by both HIAC and MFI and all the tested samples met USP <787> test requirements. No specific increase or trend in subvisible particulates was observed after 12 weeks storage across all conditions and formulations.

Viscosity and osmolality were measured and results are shown in Table 25.

TABLE 25
Viscosity (at 20 °C) and Osmolality Results

| | | Osmolality |
|---|---|---|
| Formulation | Viscosity (cP) | (mOsm/kg) |
| M01 | 11.633 | 488 |
| M02 | 12.695 | 342 |
| M03 | 14.248 | 501 |
| M04 | 18.130 | 390 |

The results from this study indicated that there were no significant physical (aggregation, precipitation, denaturation) or chemical (oxidation) differences in the stability profile of all four formulations, tested at specified storage conditions (5 °C, 25 °C, 40 °C and −20 °C) for 12 weeks. Additionally, samples subjected to freeze-thaw and agitation stress demonstrated no significant change in attributes across all formulations. With the stability profiles of each formulation being equal, viscosity and osmolality levels were the deciding factors for formulation nomination. The most desirable viscosity and osmolality was obtained at 50 mg/mL sucrose and 50 mM arginine. Thus, formulation M02 (IFNb-PF 06823859 at 150 mg/mL* in 20 mM histidine, 50 mg/mL sucrose, 50 mM arginine, 0.05 mg/mL EDTA, 0.2 mg/mL polysorbate 80, pH 5.8) was determined as the most suitable formulation for both IV and SC administration (*range of 141-154 mg/mL).

Thus, formulation M02 (IFNb-PF 06823859 at 150 mg/mL* in 20 mM histidine, 50 mg/mL sucrose, 50 mM arginine, 0.05 mg/mL EDTA, 0.2 mg/mL polysorbate 80, pH 5.8) was determined as the most suitable formulation for both IV and SC administration (*range of 141-154 mg/mL).

Further modelling refines the formulation M02 (IFNb-PF 06823859 at 150 mg/mL* in 20 mM histidine, 50 mg/mL sucrose, 50 mM arginine, 0.05 mg/mL EDTA, 0.2 mg/mL polysorbate 80, pH 5.8) as being especially suitable for SC administration (*range of 141-154 mg/mL), and the formulation (IFNb-PF 06823859 at 60 mg/mL in 20 mM histidine, 50 mg/mL sucrose, 50 mM arginine, 0.05 mg/L EDTA, 0.2 mg/mL polysorbate 80, pH 5.8) as being especially suitable for IV administration, which minimizes risk of pooling of vials leading to diluted product and is less costly to produce.

### Example 7 Population Modelling Analysis

In this analysis, models were developed to describe the PKPD of PF-06823859 in healthy volunteers and DM patients; characterize the relationship between various biomarkers; and, assess dosing options for future trials.

### Study Design

The studies included in the analysis were the first in human (FIH) study in healthy volunteers and the PII/IIb study to treat DM. The FIH study included single ascending dose and multiple ascending dose groups, with PF-06823859 given by intravenous (IV) or subcutaneous (SC) routes. The DM study was divided into 3 stages, with the first 2 focusing on skin-predominant DM and the last in muscle-predominant DM; in Stage 1 (S1), subjects were randomized to placebo or 600 mg Q4W×3 IV dosing, in S2 a 150 mg dose level was added as well as placebo crossover, and in S3 placebo crossover was included but there was no 150 mg dose level.

For the IFNß assay, the lower limit of quantification (LLOQ) was 10 μg/mL. The gene signature was based on the expression of 13 genes (including IP-10 and other proteins induced by type-I interferons) and is reported as the log 2 of the average counts per million of the gene expression; it was treated as unitless for the analysis.

### Prior Knowledge and Modeling Experience

The non-compartmental analysis of the PK data in healthy volunteers demonstrated that in that population the exposure is linear and without much inter-individual variance (IIV). On graphical analysis, there was no evidence of target-mediated drug deposition (TMDD), though that may be due to the lower target concentrations in healthy volunteers.

A previous investigational drug for DM was sifalimumab, which is a mAb for IFNα (Higgs et al, 2014). A PKPD model was developed for this drug and disease state incorporating a mechanistic link between drug, IFN and gene signature (Wang et al, 2013). AMG-811 is another mAb for a type-I interferon, IFNγ, which has a PKPD model connecting drug, IFN and interferon-gamma inducible protein 10 (IP-10) (Chen et al, 2015). These models were used to inform the development of the models in the present analysis.

### Modeling: Software and Strategy

Non-linear mixed effects modeling was performed in NONMEM version 7.5.0, with some use of PsN 5.2.6 to facilitate uncertainty estimates with sampling importance resampling (SIR). Additional processing was done in R. Modeling was performed using ADVAN13 in NONMEM to solve differential equations. The fitting algorithm was FOCE with interaction so M3 could be used. All observed data were log-transformed on both sides.

Because the healthy volunteer and early exploration of the DM data did not suggest TMDD or any other influence of PD on PK, the model was fitted sequentially. The Individual PK Parameters (IPP) approach to sequential PKPD modeling was applied.

### Pharmacokinetic Model Description

The pharmacokinetic model was developed with minimal covariates (only fixed allometric constants), attempting to address all investigated routes of administration and identifying any differences between subject type. A typical structural model for mAbs is composed of two compartments and if absorption is relatively uncomplicated it can be treated as first-order. The model was constructed considering the results from healthy volunteers and the PK in non-human primates for initial estimates. Parameterization used clearance and volume macroconstants, and observations were fitted in units of ng/mL (log-transformed). Since the PD model would be fitted separately, random effects and absorption lag were tested on the SC route even though there were limited subject data to fit those estimates.

### Base Model Description

The PD base model attempted to semi-mechanistically describe the binding of IFNβ and the association of that with downstream PD observations, such as IP-10 and gene signature (GS). The system describing the model is displayed in Equation 1. There was no TMDD to address with the model, but the binding and elimination of IFNβ did use a quasi-steady state approximation. Both protein biomarkers without drug present were modeled with simple turnover models. The central concentration of the drug (Conc) was used in combination with the quasi-steady state binding constant (K_{SS}) to estimate the fraction of total IFNβ that was bound. Bound IFNβ and unbound were eliminated by internalization (with rate constant kᵢₙₜ) and innate degradation (with rate constant k_{deg}), respectively, and free/unbound IFNβ was synthesized at a rate k_{syn}, which was determined from a steady-state assumption using baseline IFNβ. Free IFNβ was modeled to increase the synthesis rate of IP-10 (k_{syn,ip}) in a linear fashion (based on E_{si p}), with an innate, IFNβ-absent synthesis rate of k*_{syn,ip}. For IP-10, k*_{syn,ip} was also determined from a steady state assumption, with k_{syn,ip} calculated from individual baseline IFNß estimates and E_{si p}. A second compartment for IP-10 was also tested, with equal transfer rate constants k_{res,ip}.

$$Conc = {A}_{drug , central} / {V}_{central} \qquad ( 1 )$$

$${IFN}_{total} = {IFN}_{bound} + {IFN}_{free}$$

$${IFN}_{bound} = {IFN}_{total} \cdot \frac{Conc}{Conc + {K}_{SS}}$$

$${k}_{syn , ip} = {k}_{syn , ip}^{*} \cdot ( 1 + {IFN}_{free} \cdot {E}_{\text{?}} )$$

$$\frac{{dIFN}_{total}}{dt} = {k}_{syn} - {k}_{deg} \cdot {IFN}_{free} - {k}_{int} \cdot {IFN}_{bound}$$

$$\frac{dIP 10}{dt} = {k}_{syn , ip} - {k}_{deg , ip} \cdot IP 10 + {k}_{res , ip} \cdot ( IP {10}_{p} - IP 10 )$$

$$\frac{dIP {10}_{p}}{dt} = {k}_{res , ip} \cdot ( IP 10 - IP {10}_{p} )$$

$$\text{?} \text{indicates text missing or illegible when filed}$$

Although an IFNβ-independent steady state baseline was fitted for IP-10, longitudinal changes in this biomarker observed in placebo subjects prompted the investigation of non-steady state (non-SS) effects. The modeling for non-SS took advantage of the system being treated as static in the drug-free steady state, and modeled the effect as a different initial concentration of IP-10 that would be defined within that state. A multiplier term was estimated which would increase or decrease the initial concentration of IP-10 compartments above or below the steady state level, so under placebo conditions the IP-10 concentration would change to return to steady state. The non-SS multiplier would also impact active treatment groups. For GS, the lack of extensive longitudinal data and small range of observations limited the complexity with which these endpoints could be modeled. As such, CS was added last in the base model development, based on observations that the observed IP-10 concentrations and blood and lesional skin GS scores shared an double reciprocal, linear relationship, and non-lesional skin GS was proportional to lesional GS. As such, all GS scores were modeled linearly from predicted IP-10 concentrations, which are described in the equations below. In the equations, the intercept (INT) is shared for lesional skin and blood GS, but the slope (SLP) of lesional skin (SLP_{GSL}) calculated as a factor of the slope for blood GS (SLPGSB), f_{L-B}. The proportion of lesional skin CS that is accounted for in non-lesional skin is modeled with parameter Prop_{N-L}.

$${GS}_{blood}^{- 1} = {SLP}_{GSB} \cdot IP {10}^{- 1} + {INT}_{GS} \qquad ( 2 )$$

$${SLP}_{GSL} = {SLP}_{GSB} \cdot {f}_{L \text{?} B}$$

$${GS}_{\text{?} skin}^{- 1} = {SLP}_{GSL} \cdot IP {10}^{- 1} + {INT}_{GS}$$

$${GS}_{\text{?} skin} = {GS}_{\text{?} skin} \cdot {Prop}_{N - L}$$

$$\text{?} \text{indicates text missing or illegible when filed}$$

As with the PK submodel, the PD model was fitted with observations and related parameters in typical units (pg/mL for IFNβ and IP-10, unitless for CS), so any unit conversion was performed within the model code. Within the model, drug and IFNß concentrations were converted to nanomolar units using their accepted values for molecular weight.

### Random Effects Model Development

Random effects were used to model IV and residual unexplained variance (RUV). For IIV, random effects were modeled as lognormally distributed about the fixed effects parameter, as described below for individual parameter Kᵢ with fixed effect estimate θ_{K} and random effect estimate ηᵢ from a distribution with mean 0 and variance ω². In the model, IV parameters were mu-referenced to enhance stability, and any transformations necessary to preserve the referencing were made within the code so this construction is still valid.

Kᵢ=θ_{K}·exp(ηᵢ) (3)

RUV was modeled using additive and/or proportional variability, which was used to calculate the standard deviation (W) of each individual observation at any given time (Obsᵢⱼ for individual i at time j), provided a prediction (Preᵢⱼ). For additive RUV of log-transformed data, a first-order approximation was applied. In the equation below, Obsᵢⱼ is the modeled dependent variable rather than the actual observed result. For M3, the dependent variable was the likelihood (Like) estimated by the cumulative distribution function CD that the predicted observation is below the LLOQ (both transformed).

$${Pre}_{ij , log} = log ( {Pre}_{ij} ) \qquad ( 4 )$$

$${W}_{ij} = \sqrt{{RUV}_{prop}^{2} + {( \frac{{RUV}_{add}}{{Pre}_{ij}} )}^{2}}$$

$${Obs}_{ij} = {Pre}_{ijlog} + {W}_{ij} \cdot 𝒩 ( 0 , 1 )$$

$${Like}_{\text{?}} = Φ ( \frac{{LLOQ}_{log} - {Pre}_{\text{?}}}{{W}_{ij}} )$$

$$\text{?} \text{indicates text missing or illegible when filed}$$

The inclusion of random effects was guided by model diagnostics and the retention of those random effect parameters was guided by the identifiability and shrinkage of the parameter. Since there was a compelling need to test random effects on certain parameters that would not be identifiable for many individuals (i.e., for these individuals, the empirical Bayes estimates (EBE) would be 0), shrinkage was calculated based only on non-zero values. Values that were very low (<10−6) but not zero were still included in the shrinkage estimate.

### Inclusion of Covariates and Final Model Development

Covariates were added generally following a stepwise procedure. This process was followed with additional empiricism where indicated by clearly observable or theoretical differences in the parameter values between healthy and DM subjects, such as those affecting baselines for IFNβ, IP-10 and non-SS effects. Because there were few patients, extensive mapping of covariates could not be performed, and primarily those describing the differences between DM and healthy subjects were explored. Covariates were typically included in forward steps with an a of 0.01, and were retained in backwards elimination with an a of 0.001. The percent change in the standard deviation of the IV estimates was also considered as an additional check for covariate inclusion.

For the baselines, multipliers of healthy subject baseline and IIV (Mb and Mv) were fitted simultaneously for DM subjects as shown below, with 2 degrees of freedom considered in the likelihood ratio test. All of these covariate pairs were carried forward to stepwise inclusion since it was suspected controlling for major differences would facilitate testing of other potential covariates. Backwards elimination of the multipliers included sharing multipliers between baselines.

Mb_{dm}=exp(θ_{dm}) or 1+θ_{dm}

Mv_{dm}=θ_{var,dm}

Baseₕᵥ=θ_{base}

Base_{i,hv}=Baseₕᵥ·exp(ηᵢ)

Base_{i,dm}=Mb_{dm}·Baseₕᵥ·exp(Mv_{dm}·ηᵢ) (5)

Other covariates explored were modeled in a more standard fashion. Categorical covariates were fitted as a percentage change to the typical parameter value, and continuous covariates were modeled with a power relationship normalized to the median or a standard central estimate.

### Outliers

Outliers were suspected for observations with conditional weighted residuals (CWRES) greater than 6, or normalized prediction distribution error (NPDE) greater than 2. Outliers were removed if they were found to be influential in parameter estimation (greater than 10% difference with outlier present).

### Assessment of Model Adequacy (Goodness of Fit)

Standard diagnostic plots and indexes were examined to assess goodness of fit. These plots include population and individual prediction plots, and distributions, correlations and trends for EBEs and residuals. Condition number was used to assess colinearity, setting an upper boundary at 400. Wherever feasible, random effects were only included if shrinkage was less than 30%. Minimal bootstraps were used for model stability assessments. For uncertainty estimation, SIR was used with 1.2-fold inflation and PsN 5.2.6 default iterations and sample/resample ratios.

### Assessment of Model Predictive Performance (Validation)

Predictive performance was assessed by visual predictive checks (VPCs). Each VPC was associated with 1000 simulated datasets. For PK, these were stratified by first dose, final dose (treated as steady state), and subject type. For IFNβ and IP-10, observations were stratifed by subject type and (for IFNβ), % BLQ prediction was treated as a categorical endpoint. Gene signature was stratified by site for the longitudinal observations. Both skin gene signatures were jointly assessed by VPCs of gene-set improvement (GSI), defined below; this index normalizes the decrease in lesional (I) skin GS from baseline to end of study with the difference between non-lesional (nl) and lesional at baseline (mean is used to account for missing non-lesional skin GS).

### Simulations

Simulations were based on 500 simulated trails involving 100 simulated subjects each unless otherwise noted. The following simulations were performed:

GS=100·(GS_{l,skin,end}−GS_{l,skin,base})/(\sqrt{GS_{nl,skin,base}−GS_{l,skin,base}}) (6)
Comparisons of key endpoints after a simulated single 600 mg dose with a 70 kg healthy subject for visualizing covariate effects.
Placebo, 150 mg IV Q4W×3, and 600 mg IV Q4W×3 observed for a year.
Doses ranging from 10 mg to 600 mg, given at 2 through 18 weeks apart, for 1 to 3 doses (these were only 175 trials per scenario).
Dosing in adolescents, comparing 600 mg IV Q4W×3 and a comparable weight-based dosing of 9 mg/kg IV Q4W×3 (175 trials per scenario, 300 subjects per trial).

Simulated adolescent dosing populations were generated by sampling from weights for children aged 12-17 years based on the CDC weight charts LMS parameters. The primary goal of the adolescent simulations was to estimate the effect of a weight cutoff and weight-based dosing on exposure. Thus, the distributions of steady-state AUC (AUCτ), and maximum concentration (Cmax) ratios of all simulated subjects to those of a 70 kg DM subjects for various weight cutoffs for exclusion was considered. Additionally, the percent of subjects with ratios greater than 2 given a range of cutoffs for dose type (below which receive weight-based dosing, above receiving fixed adult dosing) was also considered.

### Results

### Observed Data

While the total dataset represents a range of demographics, there were disparities between the healthy and DM subjects which limited investigations of model covariates. Baseline IFNß was missing or BLQ in nearly all subjects. The PK appeared to be similar between healthy and DM subjects, where comparisons were possible. In DM, the IFNß response was associated with high variability, and most placebo recipients did not have measurable levels. IFNβ concentrations are missing for S2 and S3 at the time of the analysis. There was an apparent placebo effect on IP-10 (reducing levels over time), and overlap between the 150 mg and 600 mg response.

### Pharmacokinetic Model Results

Though the absorption was modeled with some complexity considering the limited sample with contributing data, it was useful to limit the amount of information explained with the Vc random effects, which shrunk by the inclusion of random effects on first-order absorption rate constant (ka); absorption lag had minimal impact on random effects (though it increased the estimates when introduced without random effects on ka), but substantially improved the Akaike information criterion (AIC). Similarly, the allometric constants did not greatly reduce either AIC or random effect estimates, but since they are fixed and offer information to extrapolate to different ages, they were included in the final model. Anti-drug antibodies were also assessed as a time-varying covariate on CL, but did not show any significant effect warranting inclusion.

The final PK model parameters are listed in Table 26, including SIR estimates of uncertainty. The model showed adequate diagnostics and no strong trends indicating a need for additional covariates or a simultaneous PKPD model.

TABLE 26
Final Pharmacokinetic Model Parameter estimates

| Parameter | Value | RSE | SIR Median | SIR 95% CI | SHR |
|---|---|---|---|---|---|
| Objective Function Value | −2904.8 | | | | |
| Condition Number | 7.7 | | | | |
| Systemic Clearance, θ_{CL} (L/hr) | 0.00673 | 2.93 | 0.00673 | (0.00635, 0.00708) | |
| Baseline Weight (kg) on CL | 0.75 | Fixed | NA | NA | |
| Central Volume, θ_{VC} (L) | 3.05 | 3.34 | 3.06 | (2.86, 3.25) | |
| Baseline Weight (kg) on VC | 1 | Fixed | NA | NA | |
| Intercompartmental Clearance, θ_{Q} | 0.0196 | 8.36 | 0.0196 | (0.0166, 0.0229) | |
| (L/hr) | | | | | |
| Baseline Weight (kg) on Q | 0.75 | Fixed | NA | NA | |
| Peripheral Volume, θ_{VP} (L) | 2.53 | 2.87 | 2.55 | (2.4, 2.69) | |
| Baseline Weight (kg) on VP | | Fixed | NA | NA | |
| Subcutaneous Absorption Rate | 0.0106 | 21.5 | 0.0108 | (0.00743, 0.0155) | |
| Constant, θ_{KA} (hr⁻¹) | | | | | |
| Subcutaneous Bioavailability, θ_{BIOAV} | 3.1 | 5.25 | 73.2 | (66.2, 80.9) | |
| (%) | | | | | |
| Subcutaneous Absorption Lagtime, | 0.933 | 5.01 | 0.929 | (0.844, 0.982) | |
| θ_{ALAG} (hr) | | | | | |
| Proportional RUV, θ_{RUVPRO} (% CV) | 19.7 | 1.94 | 19.7 | (19, 20.5) | |
| Additive RUV, θ_{RUVADD} (ng/mL) | 133 | 16.6 | 134 | (94.9, 181) | |
| IIV on CL, ω²_{IIVCL} (% CV) | 29.4 | 14.2 | 29.4 | (25.6, 33.9) | 11.2 |
| IIV on VC, ω²_{IIVVC} (% CV) | 32 | 15.9 | 32.1 | (27.4, 37.5) | 14.2 |
| CL, VC Covariance, ω [symbol] _{CL VC} | 0.0416 | 25 | 0.0408 | (0.025, 0.0617) | |
| IIV on KA, or ω²_{IIVKA} (% CV) | 81.6 | 43.7 | 84.4 | (54, 145) | −0.912 |
| RUV, σ_{PK} | 1 | Fixed | | | 1.12 |

[symbol]

indicates data missing or illegible when filed

% CV values for ω² estimates \sqrt{exp(ω²)∞1}. Shrinkages are calculated only from EBEs greater than 0.

### Base Model Results

The base PKPD model was able to capture all the endpoints of interest with good conditioning, although the uncertainty and IIV in the parameters was high (Table 27). The high uncertainty is partially attributable to the large number of BLQ observations and the distribution of EBEs show that the IIVs for IFNβ and IP-10 are both seemingly bimodal. The model was moderately stable, with 67% successfully minimizing on a limited bootstrap (N=200).

TABLE 27
Base Pharmacodynamic Model Parameter Estimates

| Parameter | Value | RSE | SHR |
|---|---|---|---|
| Objective Function Value | −1521.6 | | |
| Condition Number | 21.6 | | |
| IFNβ Baseline, θ_{IFNBASE} (pg/mL) | 0.104 | 42.5 | |
| IFNβ Degradation Rate Constant, θ_{IFNKDEG} (d⁻¹) | 155 | 29.4 | |
| Quasi-steady state Binding Constant, θ_{KSS} (pM) | 120 | 35.1 | |
| Bound IFNβ Elimination Rate Constant, θ_{IFNKINT} (d⁻¹) | 0.522 | 33.9 | |
| IFNβ Proportional RUV, θ_{IFNRUVPRO} (% CV) | 87.9 | 7.06 | |
| IP-10 Baseline, θ_{IPBASE} (pg/mL) | 176 | 8.09 | |
| IP-10 Non-SS Baseline Multiplier, θ_{NONSSIP} | 1.58 | 8.11 | |
| IP-10 Degredation Rate Constant, θ_{IPKDEG} (d⁻¹) | 0.213 | 14.3 | |
| IFNβ Effect Slope on IP-10 Synthesis, θ_{ESLP} (mL/pg) | 0.963 | 7.99 | |
| IP-10 Second Compartment Transfer Rate Constant, θ_{IPKRES} (d⁻¹) | 0.0335 | 17.2 | |
| IP-10 Proportional RUV, θ_{IPRUVPRO} (% CV) | 30.4 | 2.92 | |
| Reciprocal Blood GS-IP-10 Slope, θ_{GSRSLOPE} | 8.72 | 6.32 | |
| Lesional Skin GS Slope Factor, θ_{GSLFACT} | 2.29 | 7.51 | |
| Reciprocal GS-IP-10 Intercept, θ_{GSINT} | 0.109 | 1 | |
| Non-lesional Proportion of Lesional Skin GS, θ_{GSNPROP} (%) | 76.8 | 3.15 | |
| Proportionality of Skin and Blood GS IIV, θ_{GSLVPROP} | 0.355 | 6.26 | |
| Blood GS Additive RUV, θ_{GSBRUVADD} | 0.612 | 4.28 | |
| Skin GS Additive RUV, θ_{GSLRUVADD} | 1.2 | 4.76 | |
| IIV on IFNβ baseline, ω²_{IFN} (% CV) | 3400 | 19.8 | 12.9 |
| IIV on IP-10 baseline, ω²_{IP10} (% CV) | 82.7 | 17.1 | 11 |
| IIV on non-SS IP-10, ω²_{NONSS} (% CV) | 73 | 20.4 | 13.4 |
| IIV on Blood GS Store, ω²_{GS} (% CV) | 92.4 | 20.2 | 9.47 |
| IFNβ RUV, σ_{IFN} | 1 | Fixed | 6.81 |
| IP-10 RUV, σ_{IP10} | 1 | Fixed | 11.8 |
| GS RUV, σ_{GS} | 1 | Fixed | 5.6 |

Repository artifact ID FI-29387931.
% CV values for ω² estimates exp(ω²) − 1. Shrinkages are calculated only from EBEs greater than 0.

### Final Model Results

The final model demonstrated improved parameter precision over the base model and the high IV parameters were mostly explained by the added covariates (Table 28). The non-SS fixed effect predicts that (under placebo conditions for both) the average healthy volunteer will experience an increase in IP-10 over the course of a study and the average DM patient will experience a decrease in IP-10, rather than a typical decrease in any subject predicted by the base model.

TABLE 28
Final Pharmacodynamic Model Parameter Estimates

| Parameter | Value | RSE | SIR Median | SIR 95% CI | SHR |
|---|---|---|---|---|---|
| IIV on non-SS IP-10, ω²_{NONSS} (% CV) | 18.3 | 36.9 | 18.6 | (13.3, 24.7) | 20.9 |
| DM Mulitplier to IFNβ, IP-10 and non-SS | 3.33 | 16.7 | 3.28 | (2.45, 4.3) | |
| IIV, θ_{DMVAR} (fold) | | | | | |
| IIV on Blood GS Slope, ω²_{GS} (% CV) | 91.4 | 27.9 | 93.1 | (66.8, 132) | 9.78 |
| IFNβ RUV, σ_{IFN} | 1 | Fixed | | | 2.46 |
| IP-10 RUV, σ_{IP10} | 1 | Fixed | | | 10.5 |
| GS RUV, σ_{GS} | 1 | Fixed | | | 5.55 |

Repository artifact ID FI-29405634.
% CV values for ω² estimates exp(ω²) − 1. Shrinkages are calculated only from EBEs greater than 0.

### Final Model Predictive Performance

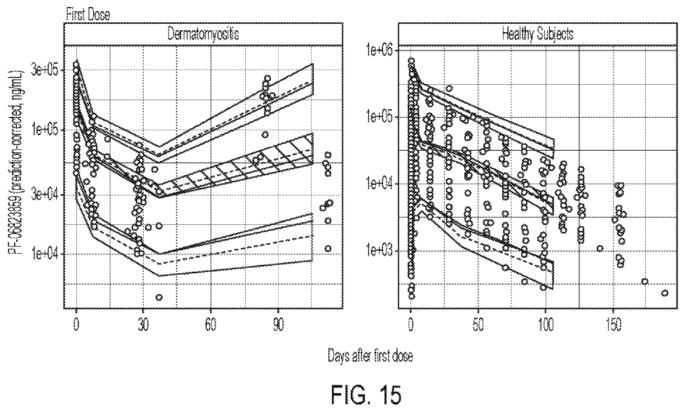

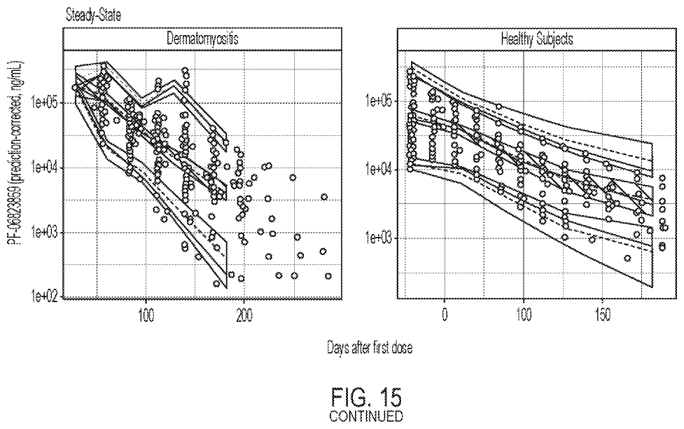

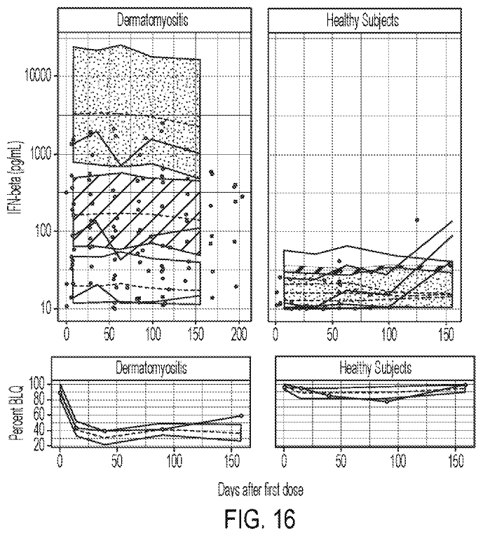

The VPCs demonstrate good predictive performance for the PK (FIG. 15) and pharmacodynamic (PD) in both healthy volunteers and patients. For IFNβ, the variability is captured well and the % BLQ over time is predicted well within a narrow distribution (FIG. 16). The early trends in IP-10 are not fully captured by the model, but the overall timecourses in all dosing levels are captured within the predicted variability. GS and GSI were predicted well, although baseline lesional skin CS was under-predicted for the 600 mg group.

### Simulations

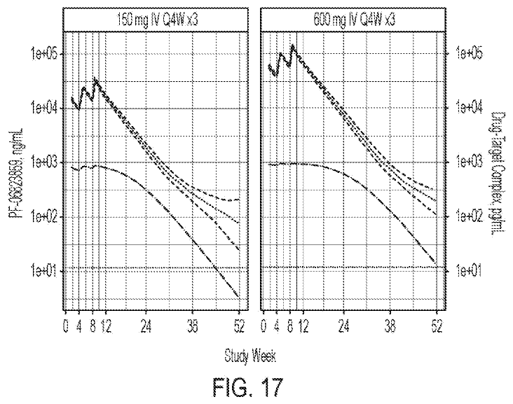

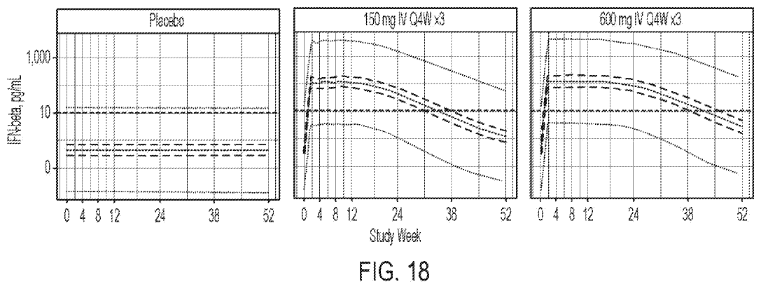

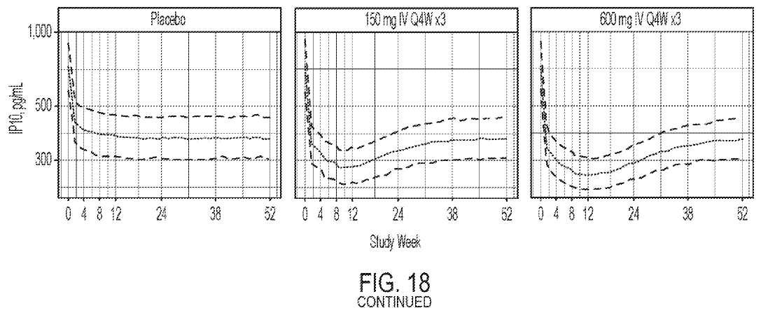

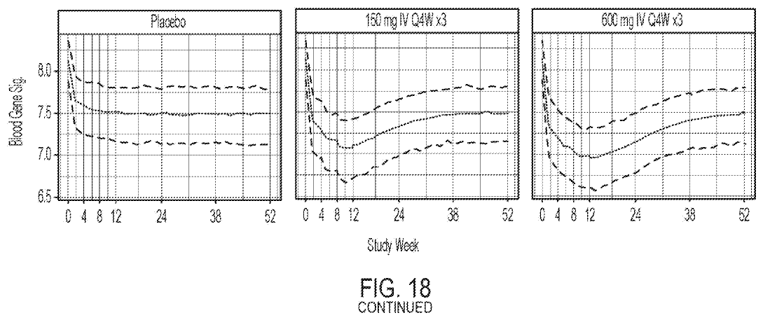

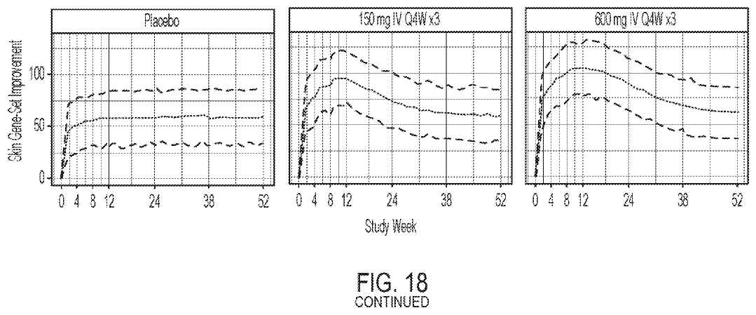

The simulations illustrate the saturation of IFNβ binding at the studied dosing levels in DM subjects (FIG. 17). From FIG. 18, it is unlikely that placebo or baseline IFNβ concentrations will be observable for DM subjects at a LLOQ of 10 μg/mL; the figures also show overlap between the other PD endpoints, with a slight point effect advantage at 600 mg. Return to steady state is predicted to occur within a year for biomarkers, though long-term extension studies are necessary to verify this prediction.

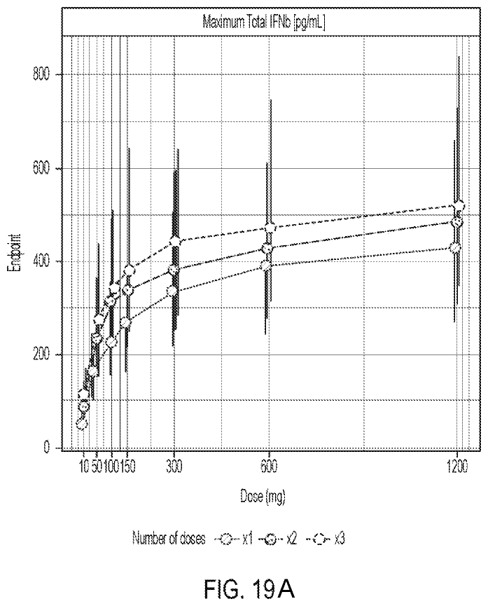

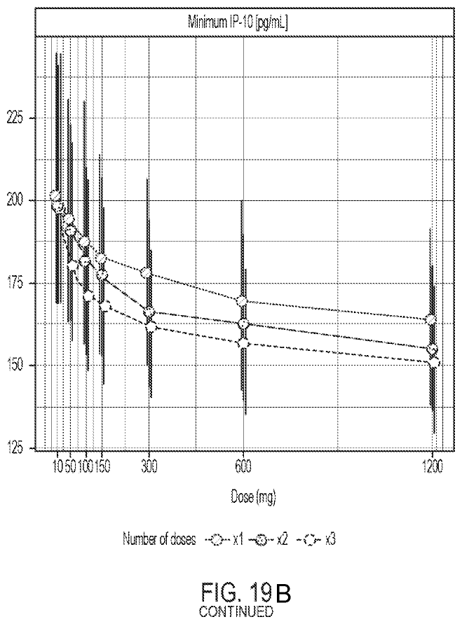

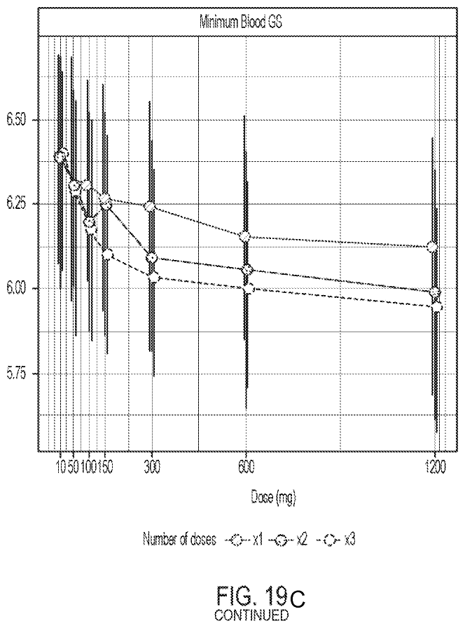

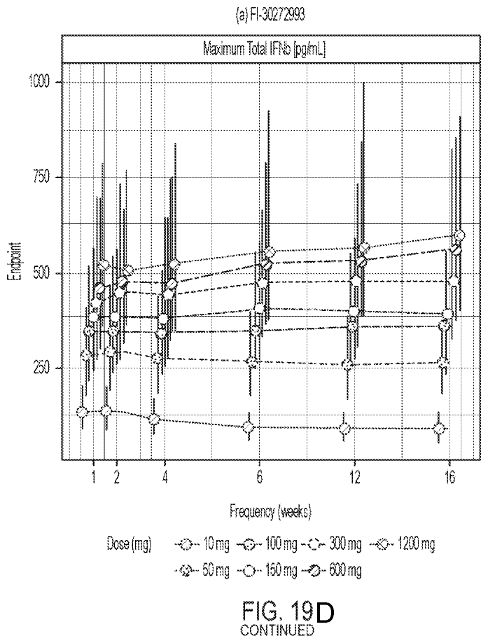

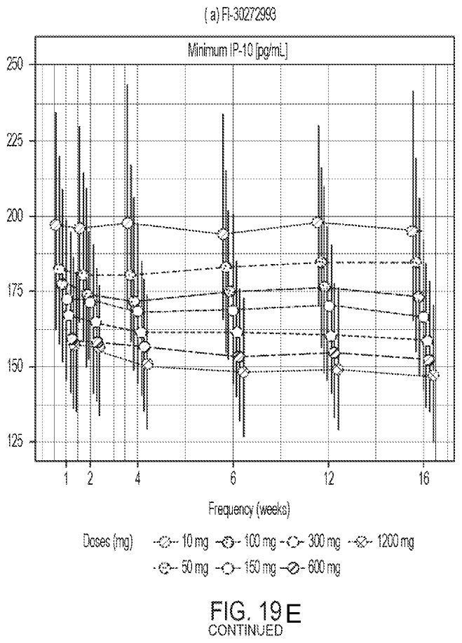

The ranged simulations show higher PD response with increased dose and number of doses, with mixed effect of increased frequency. While there is significant overlap in these measures, it appears that total cumulative dose and exposure still has a proportional relation to key biomarker responses up to 1200 mg (FIG. 19). There was less of a trend in frequency than there was for dose and number of doses.

### Adolescent Simulations

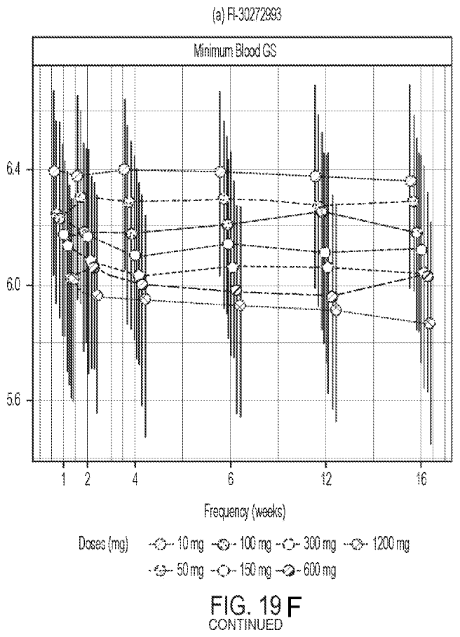

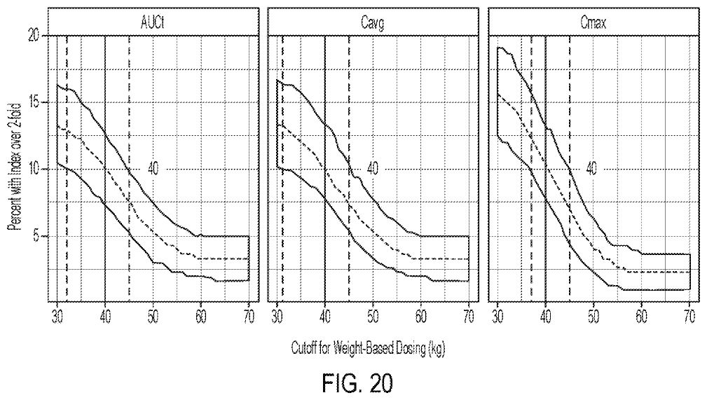

Dosing in adolescents supports a weight cutoff either for exclusion or for weight-based dosing (FIG. 20) of 40 kg, as this cutoff would be associated with fewer than 10% of subjects predicted to have 2-fold higher exposure than adults. If flat dosing were used across all likely weight ranges (to a simulated minimum of 22 kg), fewer than 20% of patients are predicted to have a two-fold increased exposure.

### Discussion

The models developed here provide an efficient and flexible description of the PK and PD of PF-06823859. The drug and target are modeled using standard quasi-steady state approximations, with other biomarkers modeled as downstream effects of IFN concentrations, which has the strength of being a semi-mechanistic approach. As such, the model is able to infer IFNβ concentrations when they are missing as long as subjects have at least IP-10 concentrations available, which also allows GS to be predicted (for skin-predominant DM, at least).

The ability to describe both non-patient, healthy subjects and DM patients is valuable given the rarity of the disease state under investigation. While demographic disparities limited extensive covariate assessment in this analysis, additional healthy subject data from other Phase I trials, and upcoming data from the end of C0251002, the extension study and Phase Ill may facilitate those investigations. From this analysis, it appears IV PK is very consistent across disease states, and major PD differences are addressed; there are no strong signals of other demographic effects, but laboratory-based values such as creatinine clearance and baseline albumin may need further analyses. Finally, while immunogenicity does not seem to have a strong effect nor high incidence, it may be a situation limited to these subject types and/or studies and so more investigation will be required as additional data are available.

Models were used to inform dosing decisions for the Phase III trial, which is expected to expand inclusion to adolescents and patients with other non-DM IIMs. The simulations demonstrate that despite apparent saturation of IFNβ coverage even at lower doses, key biomarker responses are predicted to continue in a direction of improvement at doses up to 1200 mg. They also show less sensitivity towards frequency, and 2 doses track similar point effect response compared to 3 doses. Ultimately, these subtle differences would have to be reflected in the clinical response, but would help justify less frequent and fewer doses if it were convenient for future trials. Simulations in adolescents supported a weight cutoff at 40 kg, however PK sampling in this population will be essential to verify the accuracy of these predictions. For adolescents, the threshold assessment was based on limiting a 2-fold increase in exposure, but the no observed adverse effect level (NOAEL) in cynomolgus monkeys was 25,600,000 ng/mL for Cmax and 2,430,000,000 ng h/mL for AUCτ, so given the predicted median Cmax and AUCτ are approximately 100-fold and 15-fold less than this limit in adults.

A limitation of this analysis was the extensive number of missing IFNß concentrations were inferred from those that could be identified, which may have biased the predicted results towards skin-predominant DM. There is minimal literature to inform the dynamic differences that might effect PKPD differences in skin-predominant and classic (or muscle-predominant) DM; a recent analysis in a subset of DM patients indicates muscle-predominant subjects may have higher expression of IP-10 (among other chemo/cytokines), but the model and observations for the few S3 subjects do not reflect that trend.

### CONCLUSION(S)

A model was developed to describe the PKPD of IFNβ in healthy subjects and DM patients semi-mechanistically, while addressing major differences between the subject types.
The biomarkers could be treated as correlated in response to treatment. Non-target biomarkers were modeled as dependent upon IFNβ, in both linear (GS) and non-linear (IP-10) responses.
Impact of dose, frequency and number of doses was simulated with several options available for consideration; adolescent dosing simulations supported a weight-based dosing or exclusion cutoff of 40 kg. A flat dosing to lower adolescent weights is not expected to increase exposure beyond four-fold that of a 70 kg DM patient.

### Example 8: Population PK Model

A population pharmacokinetic (popPK) model was developed using data from healthy volunteer study (C0251001) and a phase 2 patient study (C0251002). Non-linear mixed effects modeling was performed in NONMEM version 7.5.0, with use of PsN 5.2.6 to facilitate uncertainty estimates with sampling importance resampling (SIR). Additional processing was done in R. Modeling was performed using ADVAN13 in NONMEM to use differential equations. The fitting algorithm was FOCE/Laplacian with interaction. All observed data were log-transformed on both sides.

The popPK was developed with minimal covariates (only fixed allometric constants) and is composed of two compartments with first-order absorption to represent a typical structure for mAbs (Ryman). The model was constructed considering the results from healthy volunteers and the PK in non-human primates for initial estimates. Parameterization used clearance and volume macroconstants, and observations were fitted in units of ng/mL (log-transformed). The equations and the final estimates used to describe the final popPK model are as follows:

$$CL = 0.00673 \times {( \frac{Body weight}{70} )}^{0.75} \qquad ( 1 )$$

$$Vc = 3.05 \times ( \frac{Body weight}{70} ) \qquad ( 2 )$$

$$Q = 0.0196 \times {( \frac{Body weight}{70} )}^{0.75} \qquad ( 3 )$$

$$Vp = 2.55 \times ( \frac{Body weight}{70} ) \qquad ( 4 )$$

$$( 1 ) CL , typical value for clearance ;$$

$$( 2 ) Vc , typical value for central volume ;$$

$$( 3 ) Q , typical value for intercompartmental clearance ;$$

$$( 4 ) Vp , typical value for peripheral volume .$$

### Subcutaneous Dosing Rationale

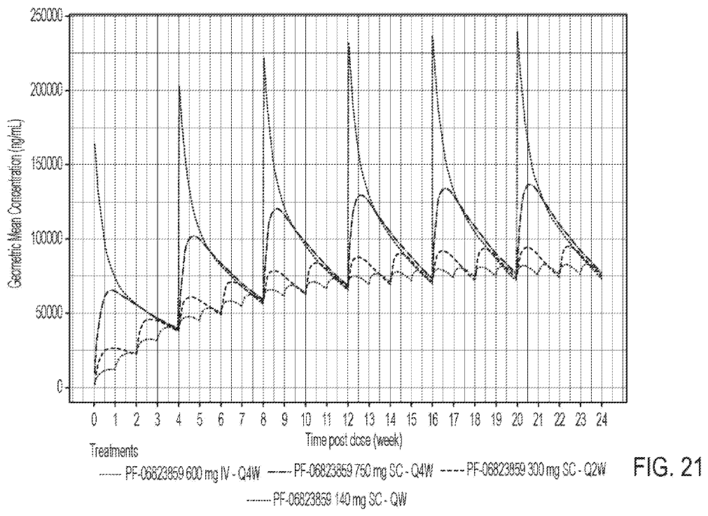

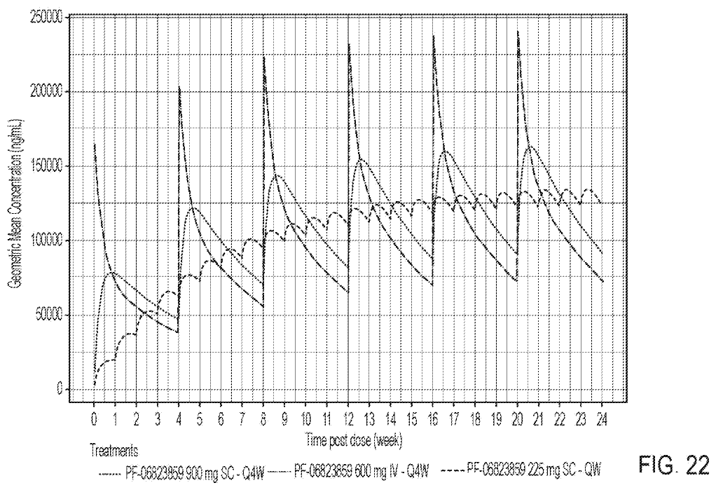

The popPK model also estimated subcutaneous bioavailability based the data from 6 healthy volunteers who received SC formulation in C0251001. The estimated subcutaneous bioavailability was 73.1%. Using the final popPK model, simulations were conducted on 100 trials, with 100 subjects per trial, and were followed up on for 24 weeks. Different subcutaneous weekly dosing regimens were tested to match the PK exposures of 600 mg IV Q4W. As shown in FIG. 21 below, at a weekly dose of 140 mg SC, trough matching is achieved between the SC dose and the 600 mg IV Q4W dosing selected for the single pivotal trial. Shown in FIG. 22 below, at a weekly dose of 225 mg SC, AUC or Caverage (Cave) matching is achieved between the SC dose and the 600 mg IV Q4W dose.

### REFERENCES

The Results of the ProDERM Study: Study Evaluating Efficacy and Safety of Octagam 10% in Patients with Dermatomyositis (Idiopathic Inflammatory Myopathy)—Study Results—ClinicalTrials.Gov
Aggarwal R, Rider L G, Ruperto N, et al. 2016 American College of Rheumatology/European League Against Rheumatism Criteria for Minimal, Moderate, and Major Clinical Response in Adult Dermatomyositis and Polymyositis: An International Myositis Assessment and Clinical Studies Group/Paediatric Rheumatology International Trials Organisation Collaborative Initiative. Arthritis Rheumatol. 2017; 69(5):898-910.
Aggarwal R, Charles-Schoeman C, Schessl J, et al. Prospective, double-blind, randomized, placebo-controlled phase Ill study evaluating efficacy and safety of octagam 10% in patients with dermatomyositis (“ProDERM Study”). Medicine (Baltimore). 2021; 100(1):e23677.
Ahmed S, Chakka S, Concha J, et al. Evaluating important change in cutaneous disease activity as an efficacy measure for clinical trials in dermatomyositis. Br J Dermatol. 2020a; 182(4):949-54.
Ahmed S, Chen K L, Werth V P. The validity and utility of the Cutaneous Disease Area and Severity Index (CDASI) as a clinical outcome instrument in dermatomyositis: A comprehensive review. Semin Arthritis Rheum. 2020b; 50(3):458-62.
Alexanderson H, Regardt M, Ottosson C, et al. Muscle Strength and Muscle Endurance During the First Year of Treatment of Polymyositis and Dermatomyositis: A Prospective Study. J Rheumatol. 2018; 45(4):538-46.
Aljebab F, Choonara I, Conroy S. Systematic Review of the Toxicity of Long-Course Oral Corticosteroids in Children. PloS one. 2017; 12(1):e0170259-e59.
Andersen L K, Davis M D. Prevalence of Skin and Skin-Related Diseases in the Rochester Epidemiology Project and a Comparison with Other Published Prevalence Studies. Dermatology. 2016; 232(3):344-52.
Baig S, Paik J J. Inflammatory muscle disease—An update. Best Pract Res Clin Rheumatol. 2020; 34(1):101484.
Batthish M, Feldman B M. Juvenile dermatomyositis. Curr Rheumatol Rep. 2011; 13(3):216-24.
Bogdanov I, Kazandjieva J, Darlenski R, et al. Dermatomyositis: Current concepts. Clin Dermatol. 2018; 36(4):450-58.
Bohan A, Peter J B. Polymyositis and dermatomyositis (second of two parts). N Engl J Med. 1975a; 292(8):403-7.
Bohan A, Peter J B. Polymyositis and dermatomyositis (first of two parts). N Engl J Med. 1975b; 292(7):344-7.
Bradford Rice J, White A, Lopez A, et al. Healthcare resource utilization and work loss in dermatomyositis and polymyositis patients in a privately-insured U S population. J Med Econ. 2016; 19(7):649-54.
Bruce B, Fries J F. The Health Assessment Questionnaire (HAQ). Clin Exp Rheumatol. 2005; 23(5 Suppl 39):S14-8.
Carpenter J R, Roger J H, Kenward M G. Analysis of longitudinal trials with protocol deviation: a framework for relevant, accessible assumptions, and inference via multiple imputation. J Biopharm Stat. 2013; 23(6):1352-71.
Chen P, Vu T, Narayanan A, Sohn W, Wang J, Boedigheimer M, Welcher A A, Sullivan B, Martin D A, Ruixo J J P et al., 2015, Pharmacokinetic and pharmacodynamic relationship of AMG 811, an anti-ifn-γ IgG1 monoclonal antibody, in patients with systemic lupus erythematosus. Pharmaceutical research vol. 32: 640-653.
Cho S K, Kim H, Myung J, et al. Incidence and Prevalence of Idiopathic Inflammatory Myopathies in Korea: a Nationwide Population-based Study. J Korean Med Sci. 2019; 34(8):e55.
Cleland J A, Venzke J W. Dermatomyositis: evolution of a diagnosis. Phys Ther. 2003; 83(10):932-45.
Cochran W G. Some Methods for Strengthening the Common χ<sup>2</sup> Tests. Biometrics. 1954; 10(4):417-51.
Dalakas M C. Polymyositis, dermatomyositis and inclusion-body myositis. N Engl J Med. 1991; 325(21):1487-98.
Dalakas M C, Hohlfeld R. Polymyositis and dermatomyositis. Lancet. 2003; 362(9388):971-82.
Dalakas M C. Immunotherapy of myositis: issues, concerns and future prospects. Nat Rev Rheumatol. 2010; 6(3):129-37.
Dalakas M C. Immunotherapy of inflammatory myopathies: practical approach and future prospects. Curr Treat Options Neurol. 2011; 13(3):311-23.
DeWane M E, Waldman R, Lu J. Dermatomyositis: Clinical features and pathogenesis. J Am Acad Dermatol. 2020; 82(2):267-81.
Dourmishev A L D L. Epidemiology of dermatomyositis. In Dermatomyositis: Advances in Recognition, Understanding and Management Edited by; 10.1007/978-3-540-79313-7. Berlin: Springer Verlag; 2009. p. 29-39.
Elman S, Hynan L S, Gabriel V, et al. The 5-D itch scale: a new measure of pruritus. Br J Dermatol. 2010; 162(3):587-93.
EMA. (2021). Jayempi EPAR Production Information.
Fasano S, Alves S C, Isenberg D A. Current pharmacological treatment of idiopathic inflammatory myopathies. Expert Rev Clin Pharmacol. 2016; 9(4):547-58.
FDA. Food and Drug Administration Patient-Focused Drug Development Guidance Public Workshop: methods to identify what is important to patients and select, develop or modify fit-for-purpose clinical outcomes assessments. October 2018.
FDA. Food and Drug Administration Public Workshop on Patient-Focused Drug Development: Guidance 4—Incorporating Clinical Outcome Assessments into Endpoints for Regulatory Decision Making. December 2019.
Feldman B M, Rider L G, Reed A M, et al. Juvenile dermatomyositis and other idiopathic inflammatory myopathies of childhood. Lancet. 2008; 371(9631):2201-12.
Findlay A R, Goyal N A, Mozaffar T. An overview of polymyositis and dermatomyositis. Muscle Nerve. 2015; 51(5):638-56.
Furst D E, Amato A A, Iorga S R, et al. Epidemiology of adult idiopathic inflammatory myopathies in a U.S. managed care plan. Muscle Nerve. 2012; 45(5):676-83.
Greenberg S A, Higgs B W, Morehouse C, et al. Relationship between disease activity and type 1 interferon- and other cytokine-inducible gene expression in blood in dermatomyositis and polymyositis. Genes Immun 2012; 13:207-13.
Huard C, Gulla S V, Bennett D V, et al. Correlation of cutaneous disease activity with type 1 interferon gene signature and interferon beta in dermatomyositis. Br J Dermatol. 2017; 176(5):1224-30.
Higgs B W, Zhu W, Morehouse C, White W I, Brohawn P, Guo X, Rebelatto M, Le C, Amato A, Fiorentino D et al., 2014, A phase 1b clinical trial evaluating sifalimumab, an anti-IFN-α monoclonal antibody, shows target neutralisation of a type i ifn signature in blood of dermatomyositis and polymyositis patients. Annals of the rheumatic diseases vol. 73: 256-262.
Huber A, Feldman B M. Long-term outcomes in juvenile dermatomyositis: how did we get here and where are we going? Curr Rheumatol Rep. 2005; 7(6):441-6.
IMACS. Disease Activity Core Set Measures. International Myositis Assessment and Clinical Studies Group (IMACS), Updated Jun. 6, 2019.
Khan S, Christopher-Stine L. Polymyositis, dermatomyositis, and autoimmune necrotizing myopathy: clinical features. Rheum Dis Clin North Am. 2011; 37(2):143-58, v.
Loarce-Martos J, Lilleker J B, Parker M, et al. Polymyositis: is there anything left? A retrospective diagnostic review from a tertiary myositis centre. Rheumatology (Oxford). 2021; 60(7):3398-403.
Lundberg I E, Tjarnlund A, Bottai M, et al. 2017 European League Against Rheumatism/American College of Rheumatology classification criteria for adult and juvenile idiopathic inflammatory myopathies and their major subgroups. Ann Rheum Dis. 2017; 76(12):1955-64.
Lundberg I E, Fujimoto M, Vencovsky J, et al. Idiopathic inflammatory myopathies. Nat Rev Dis Primers. 2021; 7(1):86.
Malik A, Hayat G, Kalia J S, et al. Idiopathic Inflammatory Myopathies: Clinical Approach and Management. Front Neurol. 2016; 7:64.
Mantel N, Haenszel W. Statistical aspects of the analysis of data from retrospective studies of disease. J Natl Cancer Inst. 1959; 22(4):719-48.
Marie M, Hafner S, Moratille S, et al. FGF2 mediates DNA repair in epidermoid carcinoma cells exposed to ionizing radiation. Int J Radiat Biol. 2012; 88(10):688-93.
McHorney C A, Ware J E, Jr., Lu J F, et al. The MOS 36-item Short-Form Health Survey (SF-36): III. Tests of data quality, scaling assumptions, and reliability across diverse patient groups. Med Care. 1994; 32(1):40-66.
Mendez E P, Lipton R, Ramsey-Goldman R, et al. U S incidence of juvenile dermatomyositis, 1995-1998: results from the National Institute of Arthritis and Musculoskeletal and Skin Diseases Registry. Arthritis Rheum. 2003; 49(3):300-5.
Na S J, Kim S M, Sunwoo I N, et al. Clinical characteristics and outcomes of juvenile and adult dermatomyositis. J Korean Med Sci. 2009; 24(4):715-21.
Noppert S J, Fitzgerald K A, Hertzog P J. The role of type I interferons in TLR responses. Immunol Cell Biol. 2007; 85(6):446-57.
NORD. (2015). Dermatomyositis.
Oddis C V, Rider L G, Reed A M, et al. International consensus guidelines for trials of therapies in the idiopathic inflammatory myopathies. Arthritis Rheum. 2005; 52(9):2607-15.
Oddis C V, Reed A M, Aggarwal R, et al. Rituximab in the treatment of refractory adult and juvenile dermatomyositis and adult polymyositis: a randomized, placebo-phase trial. Arthritis Rheum. 2013; 65(2):314-24.
Okogbaa J, Batiste L. Dermatomyositis: An Acute Flare and Current Treatments. Clin Med Insights Case Rep. 2019; 12:1179547619855370.
Oldroyd AGS, Allard A B, Callen J P, et al. A systematic review and meta-analysis to inform cancer screening guidelines in idiopathic inflammatory myopathies. Rheumatology (Oxford). 2021; 60(6):2615-28.
Oray M, Abu Samra K, Ebrahimiadib N, et al. Long-term side effects of glucocorticoids. Expert Opin Drug Saf. 2016; 15(4):457-65.
Paik J J, Casciola-Rosen L, Shin J Y, et al. Study of Tofacitinib in Refractory Dermatomyositis: An Open-Label Pilot Study of Ten Patients. Arthritis Rheumatol. 2021; 73(5):858-65.
Park J K, Mecoli C A, Alexanderson H, et al. Advancing the Development of Patient-reported Outcomes for Adult Myositis at OMERACT 2016: An International Delphi Study. J Rheumatol. 2017; 44(12):1944.
Parker M J, Chinoy H. The treatment approach of idiopathic inflammatory myopathies. EMJ. 2017; 2(4):14-18.
Patrick M, Buchbinder R, Jolley D, et al. Incidence of inflammatory myopathies in Victoria, Australia, and evidence of spatial clustering. J Rheumatol. 1999; 26(5):1094-100.
Pinal-Fernandez I, Casal-Dominguez M, Derfoul A, et al. Identification of distinctive interferon gene signatures in different types of myositis. Neurology. 2019; 93(12):e1193-e204.
Regardt M, Mecoli C A, Park J K, et al. OMERACT 2018 Modified Patient-reported Outcome Domain Core Set in the Life Impact Area for Adult Idiopathic Inflammatory Myopathies. J Rheumatol. 2019; 46(10):1351-54.
Rice J B, White A G, Scarpati L M, et al. Long-term Systemic Corticosteroid Exposure: A Systematic Literature Review. Clin Ther. 2017; 39(11):2216-29.
Rider L G, Pachman L M, Miller F W, et al. Myositis and You: A Guide to Juvenile Dermatomyositis for Patients, Families, and Healthcare Providers. The Myositis Association; 2007 2007.
Rider L G, Koziol D, Giannini E H, et al. Validation of manual muscle testing and a subset of eight muscles for adult and juvenile idiopathic inflammatory myopathies. Arthritis Care Res (Hoboken). 2010; 62(4):465-72.
Rider L G, Miller F W. Deciphering the clinical presentations, pathogenesis, and treatment of the idiopathic inflammatory myopathies. JAMA. 2011; 305(2):183-90.
Rider L G, Aggarwal R, Machado P M, et al. Update on outcome assessment in myositis. Nat Rev Rheumatol. 2018; 14(5):303-18.
Robinson A B, Reed A M. Clinical features, pathogenesis and treatment of juvenile and adult dermatomyositis. Nat Rev Rheumatol. 2011; 7(11):664-75.
Robinson A B, Hoeltzel M F, Wahezi D M, et al. Clinical characteristics of children with juvenile dermatomyositis: the Childhood Arthritis and Rheumatology Research Alliance Registry. Arthritis Care Res (Hoboken). 2014; 66(3):404-10.
Rubin D B. Multiple imputation for survey nonresponse. New York: Wiley; 1987.
Ruperto N, Martini A. Networking in paediatrics: the example of the Paediatric Rheumatology International Trials Organisation (PRINTO). Arch Dis Child. 2011; 96(6):596-601.
Ruperto N, Pistorio A, Oliveira S, et al. Prednisone versus prednisone plus ciclosporin versus prednisone plus methotrexate in new-onset juvenile dermatomyositis: a randomised trial. Lancet. 2016; 387(10019):671-78.
Ryman J T and Meibohm B, 2017, Pharmacokinetics of monoclonal antibodies. CPT: pharmacometrics & systems pharmacology vol. 6: 576-588.
Saygin D, Oddis C V. Glucocorticoids in Myositis: Initiation, Tapering, and Discontinuation. Curr Rheumatol Rep. 2022; 24(3):47-53.
See L C, Kuo C F, Chou I J, et al. Sex- and age-specific incidence of autoimmune rheumatic diseases in the Chinese population: a Taiwan population-based study. Semin Arthritis Rheum. 2013; 43(3):381-6.
Shah M, Mamyrova G, Targoff I N, et al. The clinical phenotypes of the juvenile idiopathic inflammatory myopathies. Medicine (Baltimore). 2013; 92(1):25-41.
Silverberg J I, Kwa L, Kwa M C, et al. Cardiovascular and cerebrovascular comorbidities of juvenile dermatomyositis in U S children: an analysis of the National Inpatient Sample. Rheumatology (Oxford). 2018; 57(4):694-702.
Svensson J, Arkema E V, Lundberg I E, et al. Incidence and prevalence of idiopathic inflammatory myopathies in Sweden: a nationwide population-based study. Rheumatology (Oxford). 2017; 56(5):802-10.
Symmons D P, Sills J A, Davis S M. The incidence of juvenile dermatomyositis: results from a nation-wide study. Br J Rheumatol. 1995; 34(8):732-6.
Takayanagi H, Kim S, Matsuo K, et al. RANKL maintains bone homeostasis through c-Fos-dependent induction of interferon-beta. Nature. 2002; 416(6882):744-9.
Tabata M, et al. The Type I Interferon Signature Reflects Multiple Phenotypic and Activity Measures in Dermatomyositis. Arthritis & Rheumatology. 2023, 0(0), 1-8.
Tan J A, Roberts-Thomson P J, Blumbergs P, et al. Incidence and prevalence of idiopathic inflammatory myopathies in South Australia: a 30-year epidemiologic study of histology-proven cases. Int J Rheum Dis. 2013; 16(3):331-8.
Tiao J, Feng R, Bird S, et al. The reliability of the Cutaneous Dermatomyositis Disease Area and Severity Index (CDASI) among dermatologists, rheumatologists and neurologists. Br J Dermatol. 2017; 176(2):423-30.
Vaughan H, Rugo H S, Haemel A. Risk-Based Screening for Cancer in Patients With Dermatomyositis: Toward a More Individualized Approach. JAMA Dermatology. 2022; 158(3):244-47.
Wang B, Higgs B, Chang L, Vainshtein I, Liu Z, Streicher K, Liang M, White W, Yoo S, Richman L et al., 2013, Pharmacogenomics and translational simulations to bridge indications for an anti-interferon-a receptor antibody. Clinical Pharmacology & Therapeutics vol. 93: 483-492
Ware J E, Kosinski M, Bjorner J B, et al. User's Manual for the SF-36v2® Health Survey 2. Lincoln, R I: QualityMetric Incorporated; 2007.
Ware J E, Jr., Sherbourne C D. The MOS 36-Item Short-Form Health Survey (SF-36): I. Conceptual Framework and Item Selection. Medical Care. 1992; 30(6).
Ware J E, Jr. Captopril, enalapril, and quality of life. N Engl J Med. 1993; 329(7):506-7.
Wedderburn L R, Rider L G. Juvenile dermatomyositis: new developments in pathogenesis, assessment and treatment. Best Pract Res Clin Rheumatol. 2009; 23(5):665-78.
Zu N, Wang G C. [Expression of interferon and its correlation with clinical features in idiopathic inflammatory myopathies]. Zhonghua Yi Xue Za Zhi. 2011; 91(17):1157-60.

1. A method for treating a patient with one or more conditions associated with IFNß expression in a patient, the method comprising administering to the patient an anti-IFNß antibody in a dosing regimen sufficient to improve signs and symptoms of the one or more conditions by at least 4 weeks after the start of treatment with the anti-IFNß antibody, said dosing regimen comprising a plurality of individual doses separated from each other by at least 1 week.

2. The method of claim 1, wherein the condition is one or more conditions selected from the group consisting of IIM, dermatomyositis, polymyositis, inclusion body myositis, SLE, cutaneous lupus, psoriasis, ulcerative colitis, Crohn's disease, rheumatoid arthritis, atopic dermatitis and scleroderma.

3. The method of any one of claims 1-2, wherein the condition is one or more conditions selected from the group consisting of dermatomyositis, polymyositis, inclusion body myositis, juvenile dermatomyositis, SLE, and cutaneous lupus.

4. The method of any one of claims 1-3, wherein the condition is one or more conditions selected from the group consisting of dermatomyositis, polymyositis, inclusion body myositis, and juvenile dermatomyositis.

5. The method of any one of claims 1-4, wherein one or more of the individual doses are at an amount within a range whose lower limit is selected from the group consisting of 100 mg, 150 mg, 200 mg, 250 mg, 300 mg, 350 mg, 400 mg, 450 mg, 500 mg, 550 mg, and 600 mg, and whose upper limit is selected from the group consisting of 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, and 1000 mg.

6. The method of any one of claims 1-5, wherein one or more of the individual doses are between 140 mg and 600 mg.

7. The method of any one of claims 1-6, wherein the individual doses are 140 mg.

8. The method of any one of claims 1-7, wherein the individual doses are 150 mg.

9. The method of any one of claims 1-8, wherein the individual doses are separated from each other by 1 week.

10. The method of any one of claims 1-9, wherein the individual doses are via subcutaneous injection.

11. The method of any one of claims 1-6, wherein the individual doses are between 500 and 700 mg.

12. The method of claim 10, wherein the individual doses are 600 mg.

13. The method of any one of claims 10-11, wherein the individual doses are separated from each other by 4 weeks.

14. The method of any one of claims 11-13, wherein the individual doses are via intravenous injection.

15. The method of any one of claims 1-2, wherein the dosing regimen is continued for a least a period of time selected from the group consisting of 4 weeks, 1 month, 8 weeks, 2 months, 12 weeks, 3 months, 16 weeks, 4 months, 20 weeks, 5 months, 24 weeks, 6 months, and 26 weeks.

16. The method as set forth in any one of claims 1-5, wherein the improvement in signs or symptoms relative to placebo is characterized by a clinical response, and wherein the clinical response is characterized by one or more means selected from the group consisting of
(i) a change from baseline in Manual Muscle Testing (MMT-8) score of greater than zero;
(ii) an improvement in Total Improvement Score (TIS) of greater than zero;
(iii) a change from baseline in Patient Global Assessment score of less than zero;
(iv) an improvement in absolute muscle enzyme creatinine kinase of greater than zero; and
(v) a change from baseline in Cutaneous Dermatomyositis Disease Area and Severity Index (CDASI-A) greater than zero.

17. The method according to any one of claims 1-16, wherein the patient was previously treated with at least one other medication selected from the group consisting of corticosteroids, IVIG, and an immunomodulating and immunosuppressive drug, optionally selected from the group consisting of hydroxychloroquine, azathioprine, mycophenolate mofetil, and methotrexate.

18. The method according to any one of claims 1-17, wherein the patient shows a clinical response or experiences signs or symptoms after a period of time from beginning of treatment selected from the group consisting of 4 weeks, 6 weeks, 8 weeks, 10 weeks, 12 weeks, 16 weeks, 20 weeks, 24 weeks, and 26 weeks.

19. The method according to any one of claims 1-19, wherein the anti-IFNß antibody is selected from the group consisting of
(i) an isolated antibody comprising three CDRs from the variable heavy chain region having the sequence shown in SEQ ID NO: 3 and three CDRs from the variable light chain region having the sequence shown in SEQ ID NO: 4;
(ii) an isolated antibody comprising a HCDR1 having the sequence shown in SEQ ID NO: 5, a HCDR2 having the sequence shown in SEQ ID NO: 6, a HCDR3 having the sequence shown in SEQ ID NO: 7, a LCDR1 having the sequence shown in SEQ ID NO: 8, a LCDR2 having the sequence shown in SEQ ID NO: 9, and a LCDR3 having the sequence shown in SEQ ID NO:10;
(iii) an isolated antibody comprising a variable heavy chain region having the sequence shown in SEQ ID NO: 3 and a variable light chain region having the sequence shown in SEQ ID NO: 4;
(iv) an isolated antibody comprising a heavy chain having the sequence shown in SEQ ID NO: 1 and a light chain having the sequence shown in SEQ ID NO: 2, and wherein the C-terminal lysine (K) of the heavy chain amino acid sequence of SEQ ID NO: 1 is optional;
(v) an isolated antibody comprising a VH encoded by the nucleic acid sequence of the insert of the vector deposited as CTI-AF1-VH having ATCC accession number PTA-122727 and a VL encoded by the nucleic acid sequence of the insert of the vector deposited as CTI-AF1-VL having ATCC accession number PTA-122726;
(vi) an isolated antibody comprising the VH sequence encoded by the insert in the plasmid deposited with the ATCC and having ATCC Accession No. PTA-122727;
(vi) an isolated antibody comprising the VL sequence encoded by the insert in the plasmid deposited with the ATCC and having ATCC Accession No. PTA-122726;
(vii) an isolated antibody that competes for binding with an anti-IFNß antibody comprising a variable heavy chain region having the sequence shown in SEQ ID NO: 3 and a variable light chain region having the sequence shown in SEQ ID NO: 4; and
(viii) an isolated antibody that competes for binding with an antibody comprising a VH encoded by the nucleic acid sequence of the insert of the vector deposited as CTI-AF1-VH having ATCC accession number PTA-122727 and a VL encoded by the nucleic acid sequence of the insert of the vector deposited as CTI-AF1-VL having ATCC accession number PTA-122726.

20. The method according to any one of claims 1-19, wherein the antibody is provided in an anqueous formulation, the aqueous formulation comprising
(i) the anti-IFNß antibody at a concentration of between 25 mg/mL and 200 mg·mL;
(ii) Histidine or His-HCL at a concentration of between 10 and 50 mM;
(iii) Arginine or NaCL in an amount 20-150 mM,
(iv) Sucrose or Trehalose in an amount between 20 mg/ml and 85 mg/ml;
(vi) optionally a chelator; and
(vii) at a pH of between pH 5.0 and pH 6.5.

21. Use of an anti-IFNß antibody for the preparation of a medicament for a method of treatment according to any one of claims 1-20.

22. An anti-IFNß antibody for use according to a method as claimed in any one of claims 1-20.

23. Use of an anti-IFNß antibody in the preparation of a medicament for treating a patient according to a method as claimed in any one of claims 1-20.

24. An aqueous formulation comprising:
(i) an anti-IFNß antibody at a concentration of between 25 mg/mL and 200 mg·mL;
(ii) Histidine or His-HCL at a concentration of between 10 and 50 mM;
(iii) Arginine or NaCL in an amount 20-150 mM,
(iv) Sucrose or Trehalose in an amount between 20 mg/ml and 85 mg/ml;
(v) optionally a chelator; and
(vi) at a pH of between pH 5.0 and pH 6.5.

25. An aqueous formulation for use in a method as claimed in any one of claims 1-20.


SEQUENCE LISTING (14 sequences)

SEQ ID NO: 1 - protein, 449 aa, synthetic construct
QVQLVQSGAEVKKPGSSVKVSCKASGYTFSRYWMHWVRQAPGQGLEWMGHIDPSDSYTYYNQKFKGRVTITADESTSTAYMELSSLRSEDTAVYYCARWDYGNLLFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DIQMTQSPSSLSASVGDRVTITCRTSQDIGNYLNWYQQKPGKAFKLLIYSTSRLHSGVPSRFSGSGSGTDFTLTISSLQPEDFATYYCQQGIILPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 119 aa, synthetic construct
QVQLVQSGAEVKKPGSSVKVSCKASGYTFSRYWMHWVRQAPGQGLEWMGHIDPSDSYTYYNQKFKGRVTITADESTSTAYMELSSLRSEDTAVYYCARWDYGNLLFEYWGQGTLVTVSS

SEQ ID NO: 4 - protein, 107 aa, synthetic construct
DIQMTQSPSSLSASVGDRVTITCRTSQDIGNYLNWYQQKPGKAFKLLIYSTSRLHSGVPSRFSGSGSGTDFTLTISSLQPEDFATYYCQQGIILPITFGGGTKVEIK

SEQ ID NO: 5 - protein, 10 aa, synthetic construct
GYTFSRYWMH

SEQ ID NO: 6 - protein, 17 aa, synthetic construct
HIDPSDSYTYYNQKFKG

SEQ ID NO: 7 - protein, 10 aa, synthetic construct
WDYGNLLFEY

SEQ ID NO: 8 - protein, 11 aa, synthetic construct
RTSQDIGNYLN

SEQ ID NO: 9 - protein, 7 aa, synthetic construct
STSRLHS

SEQ ID NO: 10 - protein, 9 aa, synthetic construct
QQGIILPIT

SEQ ID NO: 11 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 107 aa, synthetic construct [sequence omitted]

SEQ ID NO: 13 - DNA, 357 bp, synthetic construct
  source 1..357: mol_type = other DNA
caggtgcagctggtgcagagcggcgccgaggtgaagaagcccggcagcagcgtgaaggtgagctgcaaggccagcggctacaccttcagccggtactggatgcactgggtgcggcaggcccccggccagggcctggagtggatgggccacatcgaccccagcgacagctacacctactacaaccagaagttcaagggccgggtgaccatcaccgccgacgagagcaccagcaccgcctacatggagctgagcagcctgcggagcgaggacaccgccgtgtactactgcgcccggtgggactacggcaacctgctgttcgagtactggggccagggcaccctggtgaccgtctcgagc

SEQ ID NO: 14 - DNA, 321 bp, synthetic construct
  source 1..321: mol_type = other DNA
gacatccagatgacccagagccccagcagcctgagcgccagcgtgggcgaccgggtgaccatcacctgccggaccagccaggacatcggcaactacctgaactggtaccagcagaagcccggcaaggccttcaagctgctgatctacagcaccagccggctgcacagcggcgtgcccagccggttcagcggcagcggcagcggcaccgacttcaccctgaccatcagcagcctgcagcccgaggacttcgccacctactactgccagcaggggattattttgcccattaccttcggcggcggcaccaaggtggagatcaag